(19) United States
(12) Patent Application Publication
(10) Pub. No.: US 2026/0193643 A1
(43) Pub. Date: Jul. 9, 2026

(54) NON-VERTEBRATE CELLS FOR PERFORMING V(D)J RECOMBINATION

(71) Applicant: Georgia Tech Research Corporation, Atlanta, GA (US)

(72) Inventors: John James Blazeck, Atlanta, GA (US); Andrew Paul Cazier, Atlanta, GA (US)

(21) Appl. No.: 19/444,552

(22) Filed: Jan. 9, 2026

Related U.S. Application Data
(60) Provisional application No. 63/743,403, filed on Jan. 9, 2025.

(51) Int. Cl. C12N 15/10; C07K 16/30; C12N 15/81

(52) U.S. Cl. CPC C12N 15/1082; C07K 16/303; C07K 16/3069; C12N 15/81; C07K 2317/622; C12N 2800/102

(57) ABSTRACT

The present disclosure relates to non-vertebrate cells, including but not limited to Saccharomyces cerevisiae, engineered to express recombination proteins which allows the non-vertebrate cells to perform V(D)J recombination. The present disclosure also relates to methods of using the non-vertebrate cells to develop diverse antibodies or fragments thereof, similar to the V(D)J recombination process performed in B cells of higher mammalian organisms.

### CROSS REFERENCE TO RELATED APPLICATIONS

This application claims priority to, and the benefit of, U.S. Provisional Patent Application No. 63/743,403, filed Jan. 9, 2025, which is incorporated by reference herein in its entirety.

### STATEMENT REGARDING FEDERALLY SPONSORED RESEARCH

This invention was made with Government Support under Grant No. CA280622 awarded by the National Institutes of Health. The Government has certain rights in the invention.

### REFERENCE TO SEQUENCE LISTING

The sequence listing submitted on Jan. 9, 2026, as an .XML file entitled “10034-413US1” created on Jan. 9, 2026, and having a file size of 86,631 bytes is hereby incorporated by reference pursuant to 37 C.F.R. § 1.52(e)(5).

### FIELD

The present disclosure relates to yeast strains with the ability to perform variable-diversity-joining (V(D)J) recombination for generating antibodies or fragments thereof.

### BACKGROUND

V(D)J recombination is integral to the development of antibody diversity and proceeds through a complex DNA cleavage and repair process mediated by several proteins, including recombination-activating genes 1 and 2 (RAG1 and RAG2). V(D)J recombination occurs in all jawed vertebrates but is absent from evolutionarily distant relatives, including the yeast Saccharomyces cerevisiae. Currently, the V(D)J recombination process is limited to generating immune proteins, such as for antibodies and T cell receptors (TCRs), in higher organisms (vertebrates). Given the limitations described above, there is a need for improving and expanding the ability to develop said immune proteins.

The engineered cells or method of use thereof address these and other needs.

### SUMMARY

The present disclosure provides engineered non-vertebrate cells expressing one or more recombination activating gene (RAG) proteins which allow said engineered non-vertebrate cells to perform V(D)J recombination. The present disclosure also provides methods of using the engineered non-vertebrate cells to generate antibodies or fragments thereof. The present disclosure also provide methods of using the engineered non-vertebrate cells to screen for disease severity.

In some aspects, disclosed herein is an engineered non-vertebrate cell comprising a nucleic acid encoding one or more recombination activating gene (RAG) proteins selected from RAG1, RAG2, or a variant thereof and a recombination signal sequence (RSS), wherein the engineered non-vertebrate cell is engineered to undergo variable-diversity-joining (V(D)J) recombination to generate an antibody or a fragment thereof.

In some embodiments, the engineered non-vertebrate cell comprises a yeast cell, wherein the yeast cell includes but is not limited to a Saccharomyces cerevisiae (S. cerevisiae) yeast cell.

In some embodiments, the variant comprises a truncated RAG protein, wherein the truncated RAG protein includes but is not limited to amino acids 348-1006, 383-1006, 284-1006, 215-1008, or 1-1008 of RAG1. In some embodiments, the variant comprises a truncated RAG protein, wherein the truncated RAG protein includes but is not limited to amino acids 1-383 of RAG2.

In some embodiments, the nucleic acid encoding the RAGI protein comprises at least 70% sequence identity to SEQ ID NO: 1 or SEQ ID NO: 3. In some embodiments, the nucleic acid encoding the RAG2 protein comprises at least 70% sequence identity to SEQ ID NO: 2 or SEQ ID NO: 4.

In some embodiments, the RSS comprises a 12 base pair (bp) spacer or a 23 bp spacer. In some embodiments, the engineered non-vertebrate cell further comprises a nucleic acid encoding a High Mobility Group Box I (HMGB1) protein.

In some aspects, disclosed herein is a method of generating an antibody or a fragment thereof, the method comprising obtaining a non-vertebrate cell; introducing into the non-vertebrate cell a nucleic acid encoding one or more recombination activating gene (RAG) proteins selected from RAG1, RAG2, or variants thereof and a recombination signal sequence (RSS); and culturing the non-vertebrate cell of any preceding step under cell culture conditions to allow V(D)J recombination activity, wherein the non-vertebrate cell expresses an antibody or a fragment thereof following V(D)J recombination.

In some aspects, disclosed herein is a method of screening for disease severity caused by a mutation to a recombination activating gene (RAG) using an engineered non-vertebrate cell, the method comprising obtaining a non-vertebrate cell; engineering the non-vertebrate cell to express a mutant RAG protein, wherein the mutant RAG protein is associated with an immunodeficiency disease, and wherein the non-vertebrate cell comprises a recombination signal sequence; culturing the non-vertebrate cell of any preceding step under cell culture conditions to allow V(D)J recombination activity; and measuring V(D)J recombination activity in the non-vertebrate cell of any preceding step, wherein a decrease in V(D)J recombination activity relative to a control non-vertebrate cell indicates an increase in severity of the immunodeficiency disease.

In some embodiments, the method of any preceding aspect comprises the non-vertebrate cell being a yeast cell, wherein the yeast cell includes but is not limited to a Saccharomyces cerevisiae (S. cerevisiae) yeast cell.

In some embodiments, the method of any preceding aspect comprises the nucleic acid encoding the RAGI protein having at least 70% sequence identity to SEQ ID NO: 1 or SEQ ID NO: 3. In some embodiments, the method of any preceding aspect comprises the nucleic acid encoding the RAG2 protein having at least 70% sequence identity to SEQ ID NO: 2 or SEQ ID NO: 4.

In some embodiments, the method of any preceding aspect comprises the RSS having a 12 base pair (bp) spacer or a 23 bp spacer. In some embodiments, the one or more RAG proteins bind to one RSS with a 12 bp spacer and binds to one RSS with a 23 bp spacer.

In some embodiments, the method of any preceding aspect comprises the non-vertebrate cell further having a nucleic acid encoding a High Mobility Group Box I (HMGB1) protein. In some embodiments, the V(D)J recombination activity occurs in vivo.

### BRIEF DESCRIPTION OF FIGURES

The accompanying figures, which are incorporated in and constitute a part of this specification, illustrate several aspects described below.

FIGS. 1A, 1B, 1C, 1D, and 1E show the eGFP tagging of RAG1 and RAG2 shedding light on expression and nuclear localization. FIG. 1A shows the RAGI or RAG2 variants were tagged with eGFP on the N-terminus and integrated in BY4742-NAB2-mCherry yeast. Median eGFP fluorescence was then measured using flow cytometry after overnight culture in YPG. FIG. 1B shows the confocal microscopy images of eGFP-tagged RAGI and RAG2 protein variants in a NAB2-mCherry strain which natively highlights the nucleus. Scale bar represents 5 μm; all images are at the same scale. Gains have been adjusted from the original images to aid visual comparison. The brightfield channel for each image can be found in FIG. 8. FIG. 1C shows the nuclear localization was estimated by calculating the Pearson correlation coefficient between the RAG-eGFP and NAB2-mCherry signals of the confocal images. FIG. 1D shows the confocal microscopy images of eGFP-tagged RAGI protein variants in a NOP56-mCherry strain which natively highlights the nucleolus. As in FIG. 1B, scale bar represents 5 μm. The complete set of images can be found in FIG. 9. FIG. 1E shows the nucleolar localization was estimated by calculating the Pearson correlation coefficient between the RAG-eGFP and NOP56-mCherry signals of the confocal images. RFU=relative fluorescence units, NLS=nuclear localization signal. Statistical significance was calculated with a one-way ANOVA and Tukey test (*p<0.05, ** p<0.01, *** p<0.001, and **** p<0.0001).

FIGS. 2A, 2B, 2C, and 2D show the protein engineering increases activity of RAG-mediated, homology-assisted recombination of split antibiotic resistance gene. FIG. 2A shows the diagram of pY112-CJA-UP-H20 recombination target plasmid. Triangles represent RSSs. Cutting by RAG1/2 leads to homology-assisted DNA repair of the G418R gene that confers resistance to the antibiotic G418. FIG. 2B shows the yeast strains were engineered with both RAGI and RAG2 genes (either full-length, core, or core NLS variants) and optionally HMGB1. After 4d induction in SG-Leu media, cell cultures were plated on-Leu, G418 plates and colonies were counted after outgrowth. FIG. 2C shows the G418R recombination with yeast strains assessing the combination of full-length RAG2 with RAG1core . Cells were plated after a 4d induction. FIG. 2D shows the G418R recombination with alternate truncations of RAG1 that include more regions of the full-length protein than the initial RAG1core construct (which contains amino acids 383-1006). All strains include full-length RAG2 and HMGB1. T490A refers to a mutation in RAG2. Cells were plated after a 4d induction. R1=RAG1, R1c=RAG1core , R2=RAG2, R2c=RAG2core, NLS=nuclear localization signal. Statistical significance was calculated with a one-way ANOVA and Tukey test (ns=not significant, ** p<0.01, and **** p<0.0001).

FIGS. 3A, 3B, 3C, 3D, 3E, and 3F show that the homology and intersignal sequence strongly influence the G418 recombination assay. FIG. 3A shows the G418 recombination assay using substrates with mutated RSSs that cannot be recognized by the RAG proteins (Mut12 and Mut23) or nonstandard pairings (12+12 and 23+23). Cells were cultured for 4 days in SG-Leu prior to plating. FIG. 3B shows the G418R recombination assay showing the effect of adjusting homology length between the two halves of the G418R gene in the recombination target plasmid. Cells were plated after an 8 day (8d) inducation. FIG. 3C shows the diagram of the original intersignal region for “UP” plasmid design and the shorter variant sequences between the two halves of G418R was used. FIG. 3D shows the G418R recombination assay varying the intersignal region between RSSs. See FIG. 3C for region descriptions. Cells were plated after a 4d induction. FIG. 3E is similar to FIG. 3D in showing the G418R recombination assay testing additional intersignal regions between RSSs. Cells were plated after a 4d induction. FIG. 3F shows the G418R recombination assay using enhanced pY112-CJA-U-H20 plasmid with additional recombination strains. Recombination strains contained RAG2, HMGB1, and the specified variant of RAG1. Cells were plated after 4d induction. In FIGS. 3A, 3B, 3C, and 3D, BY4742 are wild-type cells that do no contain V(D)J recombination genes. CFU=colony formation unit. In FIGS. 3A, 3B, and 3D-3F, the data are presented as mean values±SD; n=3 biological replicates. In FIGS. 3A, 3D, and 3E, statistical significance was calculated using a two-way ANOVA and Tukey test. In FIG. 3B, significance was calculated using multiple two-sided t-tests with a Holm-Sidak correction for multiple comparisons. In FIG. 3F, statistical significance was calculated using a one-way ANOVA and Tukey test. (ns=not significant, *p<0.05, ** p<0.01, *** p<0.001, and **** p<0.0001).

FIGS. 4A and 4B show the signal joint formation and isolation using an integrated target. FIG. 4A shows the diagram of the integrated SJUE target. Cutting by RAG1/2 can lead to NHEJ repair which fuses the RSSs together and forms a signal joint. Such an event permanently removes GFP and URA3, leading to cells that do not fluoresce and are 5-FOA-resistant. FIG. 4B shows the SJUE target was integrated in both a recombination-competent (AC518) and recombination-incompetent (BY4742) strain. After an 8d induction in YPG media, cells were plated on SD, 5-FOA plates. Both eGFP-positive and eGFP-negative colonies were separately counted.

FIGS. 5A, 5B, and 5C show the recombination efficiency of pathogenic RAGI mutants in yeast correlates with activity in mammalian cells. FIG. 5A shows the genetic map of human RAGI showing the truncated core region and the location of five mutations which have been shown to cause varying degrees of immunodeficiency. FIG. 5B shows the table of human RAG1 mutants and their corresponding mutation in mouse RAG1. Additionally, the previously reported activity of the mutants in mammalian cells 63 is given along with the activity measured in yeast in this work. FIG. 5C shows the G418R recombination with pY112-CJA-U-H20 plasmid. BY4742 are wild-type yeast cells. All other strains contain RAG1core (with a mutation or without in the case of WT), RAG2, and HMGB1. Cells were plated after a 4d induction.

FIGS. 6A, 6B, 6C, 6D, and 6E show the RAG-mediated recombination of split GFP construct generates diversity; up to three colors in vivo. FIG. 6A shows the diagram of the pY112-CJCG-ES-H20 plasmid. The first portion of GFP is flanked by a 12-RSS followed by an intersignal region and then two modules each consisting of a 23-RSS, a gene fragment that creates eGFP or Sapphire, and a terminator. Each gene fragment has 20 bp of homology to the first portion of GFP. Due to the 12/23 Rule of RAG recombination, there are only two expected resolutions to recombination, one which creates eGFP and another which creates Sapphire. FIG. 6B shows the phenotype change for pY112-CJCG-ES-H20 plasmid as cells undergo recombination. Initially, all cells are non-fluorescent, but RAG-mediated recombination leads to subpopulations with eGFP or Sapphire fluorescence. FIG. 6C shows the 2-color recombination of cells with target plasmids that have a fragment order of eGFP then Sapphire (pY112-CJCG-ES-H20) or Sapphire then eGFP (pY112-CJCG-SE-H20). FIG. 6D shows the 2-color recombination with fragment order eGFP then Azurite (pY112-CJCG-EA-H20). FIG. 6E shows the 3-color recombination using a target plasmid with a fragment order of eGFP, Sapphire, then Azurite (pY112-CJCG-ESA-H20). For all tests, cells were cultured for 9d in SG-Leu media prior to analysis with flow cytometry. BY4742 are wild-type cells that do not contain V(D)J recombination genes. AC518 contains NLS-RAG1core, RAG2, and HMGB1. Statistical significance was calculated with a two-way ANOVA with repeated measures to compare colors and Tukey test (ns=not significant, *p<0.05, ** p<0.01, *** p<0.001, **** p<0.0001).

FIGS. 7A, 7B, and 7C show the two functional scFv variants can be generated with recombination and displayed. FIG. 7A shows the diagram of the pY112-CJCS-PG-H20 plasmid. AGA2 and a VH fragment are flanked by a 12-RSS followed by an intersignal region and then two modules each consisting of a 23-RSS, a gene fragment that contains a unique VHCDR3, and a terminator. RAG-mediated recombination creates either an anti-PSCA or anti-GPC3 product which can be display on the surface of the cell. FIG. 7B shows the demonstration of expected phenotype change during RAG-mediated recombination of the split-scFv substrate. Initially, the cells cannot display a functional scFv. Depending on the outcome, a fraction of cells will display either an anti-PSCA or anti-GPC3 antibody after recombination. FIG. 7C shows the scFv recombination results measured via flow cytometry. Wild-type EBY100 cells were compared to the engineered strain AC701, which expresses NLS-RAG1core , RAG2, and HMGB1. Cells were induced for 8d in SG-Trp (Galactose) or SD-Trp (Glucose), then grown for 1d SD-Trp followed by 1d in buffered SG-Trp. Due to fluorophore overlap, the cells were split into two separate staining reactions: one for PSCA and Myc and another for GPC3 and FLAG. Statistical significance was calculated with a two-way ANOVA with repeated measures to compare scFv display and Tukey test (**** p<0.0001).

FIG. 8 shows the confocal microscopy images of eGFP-tagged RAG proteins in NAB2-mCherry yeast. Cells were grown overnight in YPG prior to imaging on a Zeiss LSM-700 microscope. Each image set shows a subsection of a single z-stack slice. Images are representative of a group collected in biological triplicate (n=3). Scale bar represents 5 μm; all images are at the same scale.

FIG. 9 shows the confocal microscopy images of eGFP-tagged RAG proteins in NOP56-mCherry yeast. Cells were grown overnight in YPG prior to imaging on a Zeiss LSM-700 microscope. Each image set shows a subsection of a single z-stack slice. Images are representative of a group collected in biological triplicate (n=3). Scale bar represents 5 μm; all images are at the same scale.

FIG. 10 shows the protein sequence alignment of human and mouse HMGB1. The two sequences are nearly identical, sharing over 98% identity and differing only at two residues in the acidic tail domain.

FIGS. 11A, 11B, and 11C show the additional results from the G418R recombination assay. FIG. 11A shows the representative sequencing of homology-assisted coding joint using pY112-CJA-UP-H20 target plasmid. FIG. 11B shows the G418R recombination assay to compare the effect of codon optimization. RAG1core and RAG2core were cloned in BY4742 yeast using either the wild-type mouse sequence or a yeast codon-optimized sequence. HMGB1 was always yeast codon optimized. FIG. 11C shows the G418R recombination assay to test the effect of induction media using a strain expressing RAG1core and RAG2core. As the RAG proteins were always expressed under GAL promoters, RAG expression will only occur when the yeast are cultured in galactose. CFU=colony forming unit. In FIGS. 11B and 11C, data are presented as mean values±SD; n=3 biological replicates. In FIG. 11B, statistical significance was calculated with a two-way ANOVA and Fisher LSD test (ns=not significant, ** p<0.01). From left to right, the highlighted p values in FIG. 11B are p=0.2623 and 0.0032.

FIGS. 12A and 12B show the RAGI truncation expression and correlation with recombination activity. FIG. 12A shows the median eGFP fluorescence intensity of BY4742-NAB2-mCherry cells with RAGI truncations tagged at the C-terminus with eGFP. Cells were induced overnight in YPG prior to analysis on a flow cytometer. Data are presented as mean±SD; n=6 biological replicates. FIG. 12B shows the expression activity plotted versus recombination activity. CFU=colony forming unit. The y-axis data is taken from a, and the x-axis data is taken from FIG. 2D. A line of best-fit along with the coefficient of determination, R2, are displayed with the data. In FIG. 12A, statistical significance was calculated with a one-way ANOVA and Tukey test (** p=0.0054, **** p<0.0001).

FIGS. 13A, 13B, and 13C show the additional protein characterization of G418R recombination. FIG. 13A shows the G418R recombination assay with pY112-CJA-U-H20 (plasmid described in FIG. 3). All recombination strains had RAG1 (348-1008) (or a mutant thereof), RAG2, and HMGB1. D708N and E962Q are RAGI mutations of essential catalytic residues. R855A/R856A and R894A are RAGI mutations which have impaired hairpin formation but can still nick DNA. BY4742 and 348-1008 are repeated from FIG. 3F; both figures are derived from the same experiment. FIG. 13B shows the G418R recombination assay with pY112-CJA-UP-H20 to test the effect of the T490A mutation in RAG2. Both strains had RAG1core , HMGB1, and either normal RAG2 (left) or RAG2-T490A (right). 348-1006 is repeated from FIG. 2D as both figures are derived from the same experiment. FIG. 13C shows the G418R recombination assay with pY112-CJA-UP-H20 comparing the effect of adding a truncated human Artemis protein to a base strain, BY4742, and a recombination-competent strain containing NLS-RAG1core , RAG2, HMGB1. CFU=colony forming unit. For FIGS. 13A, 13B, and 13C, cells were plated after a 4 day (4d) induction; data are presented as mean values±SD; n=3 biological replicates. In FIG. 13B, statistical significance was calculated with an unpaired two-sided t-test, p=0.9997, and in FIG. 13C, statistical significance was calculated with a two-way ANOVA and Tukey test, p=0.4889 (ns=not significant).

FIGS. 14A, 14B, and 14C show the human RAG activity and expression. FIG. 14A shows the G418R recombination test with pY112-CJA-UP-H20 plasmid after 4d using human RAG1core , human RAG2, and HMGB1 relative to wild-type BY4742 cells. CFU=colony forming unit. FIG. 14B shows the human RAG1core or RAG2 were tagged with eGFP on the C-terminus and integrated in BY4742-NAB2-mCherry yeast and compared to the equivalent mouse proteins tagged with eGFP. Median eGFP fluorescence was then measured using flow cytometry after overnight culture in YPG. FIG. 14C shows the western blot of RAG2 or RAG2core in protein extracted from various recombination strains (left panel). Protein loading was verified by a separate gel stained with Coomassie Blue (right panel). In FIGS. 14A and 14B, data are presented as mean values±SD; n=3 biological replicates. In FIG. 14A, statistical significance was calculated with an unpaired two-sided t-test, p=0.0070, and in FIG. 14B, statistical significance was calculated with a one-way ANOVA and Tukey test; from left to right, p=0.9994 and 0.9729 (ns=not significant, ** p<0.01).

FIGS. 15A, 15B, 15C, and 15D show the additional results from altering recombination target substrate. FIG. 15A shows the time course of G418 resistant colony accumulation using the pY112-CJA-UP-H20 target plasmid in the specified strains. Cells were cultured in SG-Leu for 14d total and plated at the specified timepoints. FIG. 15B shows the G418R recombination in strain AC518 (NLS-RAG1core , RAG2, and HMGB1) comparing the effect of plasmid origin of replication (and copy number) on recombination using the “UP” spacer and 20 bp of homology. Cells were plated after a 4d induction. FIG. 15C shows the G418R recombination using the “UP” spacer and 20 bp of homology that has been integrated into the genome of AC518 or BY4742. Cells were plated after a 4d induction in SG media prior to plating on YPD G418 plates. FIG. 15D shows the G418R recombination assay using the pY112-CJA-UP-H20 target versus pY112-CJA-UP-H20alt. In the “alt” plasmid, G418R was split in a different location, and thus has a different 20 bp of homology relative to the standard target. Cells were plated after 4d induction. CFU=colony forming unit. For FIGS. 15A-15D, data are presented as mean values±SD; n=3 biological replicates. In FIGS. 15B and 15D, statistical significance was calculated using an unpaired two-sided t-test; in FIG. 15B, p=0.8966, and in FIG. 15C, p=<0.0001. In FIG. 15D, significance was calculated using a two-way ANOVA and Tukey test, p=0.2293 (ns=not significant, **** p<0.0001). Source data are provided in the Source Data file.

FIGS. 16A and 16B show the sequencing from the integrated signal joint formation assay. FIG. 16A shows the sequencing of a non-fluorescent, AC518-SJ colony grown on a 5-FOA plate. The RSSs are joined without error at the heptamer, forming a canonical signal joint.

FIG. 16B shows the sequencing of a fluorescent, AC518-SJ colony grown on a 5-FOA plate. The cryptic RSS heptamer differs at only one position from the consensus sequence, and the nonamer only differs at two nucleotides.

FIGS. 17A, 17B, 17C, and 17D shows the representative flow cytometry plots from GFP recombination. FIG. 17A shows the single-color control showing how Azurite events were gated using two channels. FIG. 17B shows the single-color control showing how Sapphire events were gated using two channels. FIG. 17C shows the single-color control showing how eGFP events were gated using two channels. FIG. 17D shows the three-color recombination with the pY112-CJCG-ESA-H20 plasmid. BY4742 are wild-type cells that do not contain V(D)J recombination genes. Cells were induced in SG-Leu media for 9d prior to flow cytometry.

FIG. 18 shows the representative flow cytometry plots from scFv recombination. Each condition was treated with two separate stains, one for PSCA/Myc and another for GPC3/FLAG. In each plot, the x-axis corresponds to antigen binding and the y-axis to peptide presentation. All plots are from strain AC701 which contains NLS-RAG1core , RAG2, and HMGB1. Cells with pY110-CJCS-PG plasmid were precultured in either SG-Trp or SD-Trp media for 8d. Then all cells, including single-scFv controls, were grown in SD-Trp for 1d followed by buffered SG-Trp for 1d to induce display. All plots were derived from forward scatter (FSC) singlet populations.

FIGS. 19A and 19B shows true coding joint formation occurring in engineered yeast without coding end homology. FIG. 19A shows the results of a G418 resistance recombination assay using target plasmid pY112-CJA-U-HOopt. Cells were plated after 2 days. Strain AC516 includes RAG1 (348-1006), RAG2, and HMGB1. Strain AC516-MRX is a modified form of AC516 that overexpresses the yeast genes MRE11, RAD50, and XRS2.

FIG. 19B shows an alignment of sequencing results from six G418-resistant colonies collected from the recombination assay. The sequences are aligned to the G418 gene. The mutation outcomes are summarized as follows: 1) insertion of alanine (A); C1-sequenced recombination; 2) conversion of noted arginine (R) to alanine (A); C3, C5, C6-sequenced recombination; and 3) insertion of alanine (A) and conversion of noted arginine (R) to glutamine (Q); C4-sequenced recombination. These mutation outcomes demonstrate true coding joint formation via V(D)J recombination.

FIG. 20 shows a longer section of the alignment of sequencing results from six G418-resistant colonies collected from the recombination assay that is also shown in FIG. 19B. The sequences are aligned to the G418 gene.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification. Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’ ‘about y,’ and ‘about z’ as well as the ranges of ‘less than x,’ ‘less than y.’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,′ and ‘greater than z.’ In addition, the phrase “about ‘x’ to ‘y’,” where ‘x’ and ‘y’ are numerical values, includes “about ‘x’ to about ‘y’.” Such a range format is used for convenience and brevity and thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

An “increase” can refer to any change that results in a greater amount of a symptom, disease, composition, condition, or activity. An increase can be any individual, median, or average increase in a condition, symptom, activity, or composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, or composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words, it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., a physician.

The term “therapeutically effective” refers to the amount of the composition used is of sufficient quantity to ameliorate one or more causes or symptoms of a disease or disorder. Such amelioration only requires a reduction or alteration, not necessarily elimination.

As used herein, the term “genetically modified” refers to a living cell, tissue, or organism whose genetic material has been altered using genetic engineering techniques. The genetic modification results in an alteration that does not occur naturally by mating and/or natural recombination. Modified genes can be transferred within the same species, across species (creating transgenic organisms), and across kingdoms. New, exogenous genes can be introduced, or endogenous genes can be enhanced, altered, or knocked out.

The term “recombinant” describes any DNA, proteins, cells, or organisms that are made by combining genetic material from two different sources. For example, a bacterial gene being inserted into a human plasmid, or human DNA construct, to create another construct that would not otherwise be found in either genome.

The term “antibody” is used in the broadest sense, and specifically covers monoclonal antibodies (including full-length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules that lack target specificity. Native antibodies and immunoglobulins are usually heterotetrameric glycoproteins of about 150,000 Daltons, composed of two identical light (L) chains and two identical heavy (H) chains. Each heavy chain has at one end a variable domain (VH) followed by a number of constant domains. Each light chain has a variable domain at one end (VL) and a constant domain at its other end.

A particular kind of chimeric antibody is a “humanized” antibody, in which the antibodies are produced by substituting the CDRs of, for example, a mouse antibody, for the CDRs of a human antibody (see e.g., PCT International Patent Application Publication No. WO 1992/22653). Thus, in some embodiments, a humanized antibody has constant regions and variable regions other than the CDRs that are derived substantially or exclusively from the corresponding regions of a human antibody, and CDRs that are derived substantially or exclusively from a mammal other than a human.

The term “antibody fragment” refers to a portion of a full-length antibody, generally the target binding or variable region. Examples of antibody fragments include Fab, Fab′, F(ab′) 2, and Fv fragments. The phrase “functional fragment or analog” of an antibody is a compound having qualitative biological activity in common with a full-length antibody. For example, a functional fragment or analog of an anti-IgE antibody is one that can bind to an IgE immunoglobulin in such a manner as to prevent or substantially reduce the ability of such molecule from having the ability to bind to the high-affinity receptor, FcERI. As used herein, “functional fragment” with respect to antibodies, refers to Fv, F (ab), and F (ab) 2 fragments. An “Fv” fragment is the minimum antibody fragment that contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Collectively, the six CDRs confer target binding specificity to the antibody. However, even a single variable domain (or half of an Fv comprising only three CDRs specific for a target) has the ability to recognize and bind the target, although at a lower affinity than the entire binding site. “Single-chain Fv” or “sFv” antibody fragments comprise the VH and VL domains of an antibody, wherein these domains are present in a single polypeptide chain. Generally, the Fv polypeptide further comprises a polypeptide linker between the VH and VL domains which enables the sFv to form the desired structure for target binding.

A “control” is an alternative subject or sample used in an experiment for comparison purposes. A control can be “positive” or “negative.”

As used herein, “enhance”, “enhanced”, “enhancement”, “enhancing”, and any grammatical variations thereof as used herein, refers to an act of intensifying, increasing, or further improving the quality, value, or extent of a biological function, composition, compound, cell, or tissue.

As used herein, “diagnose”, “diagnosed”, “diagnosing”, and any grammatical variations thereof as used herein refers to the act of the process of identifying the nature of an illness, disease, disorder, or condition in a subject by examination or monitoring of symptoms.

A “protein”, “polypeptide”, or “peptide” each refer to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like.

Reference also is made herein to peptides, polypeptides, proteins, and compositions comprising peptides, polypeptides, and proteins. As used herein, a polypeptide and/or protein is defined as a polymer of amino acids, typically of length>100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods consider conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acid sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

It is understood that the average molecular weight of one amino acid is about 110 Daltons (Da). Therefore, 10 amino acids is about 1100 Da. It is further understood that 1000 Da is the same as 1 kilodalton (kDa).

The term “variant” means a polypeptide derived from a parent polypeptide by one or more (several) alteration(s), i.e., a substitution, insertion, and/or deletion, at one or more (several) positions. A substitution means a replacement of an amino acid occupying a position with a different amino acid; a deletion means removal of an amino acid occupying a position; and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’). Therefore, for an amino acid named/numbered ‘X,’ the insertion may be at position ‘X+1’ (‘downstream’) or at position ‘X-1’ (‘upstream’).

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences-a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

A variant polypeptide may have substantially the same functional activity as a reference polypeptide. For example, a variant polypeptide may exhibit one or more biological activities associated with binding a ligand and/or binding DNA at a specific binding site.

As used herein, the term, “deletion,” also called gene deletion, deficiency, or deletion mutation, refers to part of a chromosome or a sequence of DNA being left out during DNA replication. Deletion, or gene deletions can cause any number of nucleotides to be deleted from a single base to an entire piece of chromosome.

Variants comprising deletions relative to a reference amino acid sequence or nucleotide sequence are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

As used herein, a “truncated protein” or a “truncation” refers to a protein modification, wherein a protein is shorter in length relative to the full-length protein. Generally, such truncated proteins are made when protein translation is terminated early, often due to a genetic mutation such as for example a premature stop codon (also known as a nonsense mutation) or a frameshift (which leads to loss of function, altered function, or sometimes toxicity).

“Culture” or “cell culture” is the process by which cells are grown under controlled conditions, generally outside their natural environment. After the cells of interest have been isolated from living tissue, they can subsequently be maintained under carefully controlled conditions. These conditions vary for each cell type, but generally consist of a suitable vessel with a substrate or medium that supplies the essential nutrients (amino acids, carbohydrates, vitamins, minerals), growth factors, hormones, and gases (CO2, O2), and regulates the physio-chemical environment (pH buffer, osmotic pressure, temperature). Most cells require a surface or an artificial substrate to form an adherent culture as a monolayer (one single-cell thick), whereas others can be grown free floating in a medium as a suspension culture. “Cell culture” also refers to the culturing of cells derived from multicellular eukaryotes, especially animal cells, in contrast with other types of culture that also grow cells, such as plant tissue culture, fungal culture, and microbiological culture (of microbes).

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “detect” or “detecting” refers to an output signal released for the purpose of sensing of physical phenomenon. An event or change in environment is sensed and signal output is released in the form of light.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., immunodeficiency disorder caused by a RAG mutation) The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc. In some aspects, the composition disclosed herein comprises an engineered non-vertebrate cell.

The term “kit” describes a wide variety of bags, containers, carrying cases, and other portable enclosures that may be used to carry and store solid substances, liquid substances, and other accessories necessary to test the severity of a disease caused by a RAG mutation. Such kits and their contents along with any applicable procedures may be used to provide access to testing the severity of a disease caused by a RAG mutation in accordance with the teachings of the present disclosure.

A “gene” refers to a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein.

The terms “treat,” “treating,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Treatments are administered to a subject prior to onset (e.g., before obvious signs of immunodeficiency disorders caused by a RAG mutation), during early onset (e.g., upon initial signs and symptoms of immunodeficiency disorders caused by a RAG mutation), or after an established development of immunodeficiency disorders caused by a RAG mutation.

A “pharmaceutically effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “therapeutic composition” refers to at least one substance, molecule, or compound suitable for administering to a subject, wherein the composition further includes a pharmaceutical carrier. A non-limiting example includes a therapeutic composition comprises a nucleobase-poly-amino acid carrier and a sterile water-based solution.

The term “screening” refers to a method especially used in drug discovery in which data processing/control software, liquid handling devices, and sensitive detectors can allow for quick conductions of chemical, genetic, or pharmacological tests. This process allows one to quickly recognize active compounds, antibodies, or genes that modulate a particular biomolecular pathway. The results of these processes provide starting points for drug design.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate-buffered saline solution, water, emulsions (such as an oil/water or water/oil emulsion), and/or various types of wetting agents.

Efforts have been made to ensure accuracy with respect to numbers (e.g., amounts, temperature, etc.), but some errors and deviations should be accounted for. Unless indicated otherwise, parts are parts by weight, the temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Non-Vertebrate Cells and Compositions Thereof

Variable-diversity-joining (V(D)J) recombination is a mechanism of somatic recombination generally occurring in developing lymphocytes (B cells and T cells). The mechanism involves generating a highly diverse repertoire of immunoglobulins/antibodies or T cell receptors (TCRs) by shuffling and joining Variable, Diversity, and Joining genes together forming the foundation of the adaptive immune system's ability to recognize countless pathogens. Currently, the V(D)J recombination process is limited to generating immunoglobulins/antibodies and TCRs in higher organisms, such as vertebrate animals. Therefore, there is a need to improve and expand the ability to generate vast combinatorial diversity in said antibodies and TCRs.

The present disclosure provides inducing V(D)J recombination in non-vertebrate organisms to promote combinatorial diversity in antibodies. A non-limiting example of non-vertebrate organisms includes yeast, such as for example Saccharomyces cerevisiae (S. cerevisiae) yeast. As yeast grow quickly and are a platform for antibody display, the present disclosure further expands the applicability of yeast to generate diverse antibodies.

The key enzymes involved in V(D)J recombination are the recombination activating genes 1 and 2 (RAGI and RAG2). The RAGI and RAG2 enzymes are encoded by the recombination activating genes (RAGs), whose cellular expression is restricted to developing lymphocytes, which are components of the vertebrate adaptive immune system. RAG enzymes work as multi-subunit complexes that, in general, separate, shuffle, and rejoin the VDJ genes.

Specifically, RAG enzymes function to cleave single double stranded DNA between immune protein coding segments and a flanking recombination signal sequence (RSS), thus initiating the recombination process. A “RSS” refers to a conserved sequence of noncoding DNA (in the form of a heptamer sequence (7 base pairs (bps)), a space sequence, and a nonamer sequence (9bps)) that is recognized by RAG1/RAG2 enzymes and acts to guide RAG1/RAG2 enzymes to the cleavage site between a V, D, or J gene and said RSS.

Thus, present disclosure also provides engineered non-vertebrate cells expressing RAG1 and/or RAG2 in combination with a RSS allowing said engineered non-vertebrate cells to perform V(D)J recombination.

In some aspects, disclosed herein is an engineered non-vertebrate cell comprising a nucleic acid encoding one or more recombination activating gene (RAG) proteins selected from RAG1, RAG2, or a variant thereof and a recombination signal sequence (RSS), wherein the engineered non-vertebrate cell is engineered to undergo variable-diversity-joining (V(D)J) recombination to generate an antibody or a fragment thereof.

In some embodiments, the RAGI protein comprises a full-length RAGI protein. It should be noted that human RAGI protein contains 1043 amino acids and mouse RAG1 protein contains 1040 amino acids. Thus, in some embodiments, the full length RAGI protein comprises either 1040 or 1043 amino acids.

In some embodiments, the variant comprises a truncated RAG1 protein. In some embodiments, the truncated RAG1 protein is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 amino acids shorter than the full-length RAG1 protein. In some embodiments, the truncated RAG1 protein includes but is not limited to amino acids 348-1006, 383-1006, 284-1006, 215-1008, or 1-1008 of the full length RAG1 protein.

In some embodiments, the nucleic acid encoding the RAGI protein comprises at least 70% sequence identity to SEQ ID NO: 1 or SEQ ID NO: 3. In some embodiments, the nucleic acid encoding the RAGI protein comprises at 80% sequence identity to SEQ ID NO: 1 or SEQ ID NO: 3. In some embodiments, the nucleic acid encoding the RAGI protein comprises at 90% sequence identity to SEQ ID NO: 1 or SEQ ID NO: 3. In some embodiments, the nucleic acid encoding the RAG1 protein comprises at 95% sequence identity to SEQ ID NO: 1 or SEQ ID NO: 3. In some embodiments, the nucleic acid encoding the RAGI protein comprises at 99% sequence identity to SEQ ID NO: 1 or SEQ ID NO: 3. In some embodiments, the nucleic acid encoding the RAGI protein comprises SEQ ID NO: 1 or SEQ ID NO: 3.

In some embodiments, the RAG2 protein comprises a full-length RAG2 protein. It should also be noted that human and mouse RAG2 contain 527 amino acids. Thus, in some embodiments, the full length RAG2 protein comprises 527 amino acids.

In some embodiments, the variant comprises a truncated RAG2 protein. In some embodiments, the truncated RAG2 protein is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 amino acids shorter than the full-length RAG2 protein. In some embodiments, the truncated RAG2 protein includes but is not limited to amino acids 1-383 of RAG2.

In some embodiments, the nucleic acid encoding the RAG2 protein comprises at least 70% sequence identity to SEQ ID NO: 2 or SEQ ID NO: 4. In some embodiments, the nucleic acid encoding the RAG2 protein comprises at least 80% sequence identity to SEQ ID NO: 2 or SEQ ID NO: 4. In some embodiments, the nucleic acid encoding the RAG2 protein comprises at least 90% sequence identity to SEQ ID NO: 2 or SEQ ID NO: 4. In some embodiments, the nucleic acid encoding the RAG2 protein comprises at least 95% sequence identity to SEQ ID NO: 2 or SEQ ID NO: 4. In some embodiments, the nucleic acid encoding the RAG2 protein comprises at least 99% sequence identity to SEQ ID NO: 2 or SEQ ID NO: 4. In some embodiments, the nucleic acid encoding the RAG2 protein comprises SEQ ID NO: 2 or SEQ ID NO: 4.

The RSS comprises a conserved heptamer sequence (7 base pairs (bps)), spacer sequences, and a conserved nonamer sequence (9bps) that are adjacent to the V, D, and J sequences. In some embodiments, the RSS comprises a 12 base pair (bp) spacer and/or a 23 bp spacer. The RAG1/RAG2 enzymes form a complex with the RSS by following a 12-23 rule to join the V, D, and J segments by pairing a 12-bp spacer RSS to a 23-bp spacer RSS. In some embodiments, the RSS of any preceding aspect comprises any one of SEQ ID NO: 6, SEQ ID NO: 7, SEQ ID NO: 8, SEQ ID NO: 9, SEQ ID NO: 10, SEQ ID NO: 11, or a combination thereof.

The High Mobility Group Box 1 (HMGB1) is a versatile protein acting as a DNA chaperone in the nucleus functioning to maintain chromosome structure. In relation to V(D)J recombination, the HMGB 1 also functions as a cofactor to cooperatively bind the RAG1/RAG2 proteins to bend and stabilize the RSSs resulting in more efficient V(D)J recombination. In some embodiments, the engineered non-vertebrate cell further comprises a nucleic acid encoding a High Mobility Group Box I (HMGB1) protein. In some embodiments, the engineered non-vertebrate cell further comprises a nucleic acid comprising SEQ ID NO: 5.

In some embodiments, RAG1, RAG2, RSS, HMGB1, and any variants thereof are introduced into the non-vertebrate cell using any expression vector known in the art. As such, the nucleic acid sequences encoding RAG1, RAG2, RSS, HMGB1, and any variants thereof are inserted into the nucleic acid sequences of the expression vector. Non-limiting examples of expression vectors include plasmids, viral vectors, viruses, cosmids, and artificial chromosomes. Further, said expression vectors are integrated into the genome of the non-vertebrate cell. As used herein, the term “integrated” used in reference to an expression vector (e.g., a plasmid or viral vector) means the expression vector, or a portion thereof, is incorporated (physically inserted or ligated) into the chromosomal DNA of a host cell. The plasmids disclosed herein refer to small, circular DNA molecules originating from bacteria that replicate independently from the main chromosome or chromosomes of a non-vertebrate cell.

The present disclosure provides numerous plasmid strains carrying the nucleic acid sequences encoding RAG1, RAG2, RSS, HMGB1, and any variants thereof as can be seen in Table 1. Plasmids, including those disclosed in Table 1, also offer additional benefits including, but not limited to resistance to antibiotics, virulence factors, and the ability to degrade toxins helping the host cell to survive harsh environments. Therefore, the present disclosure describes stable, viable non-vertebrate cells capable of performing V(D)J recombination to promote generation of diverse antibody and/or TCR repertoires.

Methods of using non-vertebrate cells Herein, RAGI and RAG2 are incorporated into yeast resulting in the recombination ability demonstrating successful formation of coding joints. The platform disclosed herein further assays the severity of several disease-causing RAGI mutations and is used to simultaneously generate up to three unique fluorescent proteins or two distinct antibody fragments starting from an array of nonfunctional gene fragments, which is the first ever demonstration of in vivo generation of combinatorial genetic diversity in a non-vertebrate cell. Thus, the present disclosure also provides methods of using the engineered non-vertebrate cells to generate antibodies, TCRs, or fragments thereof. The present disclosure also provide methods of using the engineered non-vertebrate cells to screen for disease severity.

In some aspects, disclosed herein is a method of generating an antibody, a TCR, or a fragment thereof, the method comprising obtaining a non-vertebrate cell; introducing into the non-vertebrate cell a nucleic acid encoding one or more recombination activating gene (RAG) proteins selected from RAG1, RAG2, or variants thereof and a recombination signal sequence (RSS); and culturing the non-vertebrate cell of any preceding step under cell culture conditions to allow V(D)J recombination activity, wherein the non-vertebrate cell expresses an antibody, a TCR, or a fragment thereof following V(D)J recombination.

In some embodiments, the method comprises generating an antibody of a different species including but not limited to human, mouse, rat, rabbit, donkey, goat, and horse. In some embodiments, the method comprises generating antibodies that recognize target proteins from a different species, including but not limited to human, mouse, rat, rabbit, donkey, goat, and horse. In some embodiments, the method comprises generating an antibody of any isotype, such as for example IgG, IgM, IgA, IgE, IgD, and any isotype subclasses thereof.

In some aspects, disclosed herein is a method of screening for disease severity caused by a mutation to a recombination activating gene (RAG) using an engineered non-vertebrate cell, the method comprising obtaining a non-vertebrate cell; engineering the non-vertebrate cell to express a mutant RAG protein, wherein the mutant RAG protein is associated with an immunodeficiency disease, and wherein the non-vertebrate cell comprises a recombination signal sequence; culturing the non-vertebrate cell of any preceding step under cell culture conditions to allow V(D)J recombination activity; and measuring V(D)J recombination activity in the non-vertebrate cell of any preceding step, wherein a decrease in V(D)J recombination activity relative to a control non-vertebrate cell indicates an increase in severity of the immunodeficiency disease.

In some embodiments, the V(D)J recombination activity of any preceding aspect occurs in vivo.

In some embodiments, the method of any preceding aspect comprises the nucleic acid encoding the RAG1 protein having at least 70% sequence identity to SEQ ID NO: 1 or SEQ ID NO: 3. In some embodiments, the method of any preceding aspect comprises the nucleic acid encoding the RAG1 protein having at least 80% sequence identity to SEQ ID NO: 1 or SEQ ID NO: 3. In some embodiments, the method of any preceding aspect comprises the nucleic acid encoding the RAGI protein having at least 90% sequence identity to SEQ ID NO: 1 or SEQ ID NO: 3. In some embodiments, the method of any preceding aspect comprises the nucleic acid encoding the RAG1 protein having at least 95% sequence identity to SEQ ID NO: 1 or SEQ ID NO: 3. In some embodiments, the method of any preceding aspect comprises the nucleic acid encoding the RAGI protein having at least 99% sequence identity to SEQ ID NO: 1 or SEQ ID NO: 3. In some embodiments, the method of any preceding aspect comprises the nucleic acid encoding the RAGI protein having SEQ ID NO: 1 or SEQ ID NO: 3.

In some embodiments, the method of any preceding aspect comprises the nucleic acid encoding the RAG2 protein having at least 70% sequence identity to SEQ ID NO: 2 or SEQ ID NO: 4. In some embodiments, the method of any preceding aspect comprises the nucleic acid encoding the RAG2 protein having at least 80% sequence identity to SEQ ID NO: 2 or SEQ ID NO: 4. In some embodiments, the method of any preceding aspect comprises the nucleic acid encoding the RAG2 protein having at least 90% sequence identity to SEQ ID NO: 2 or SEQ ID NO: 4. In some embodiments, the method of any preceding aspect comprises the nucleic acid encoding the RAG2 protein having at least 95% sequence identity to SEQ ID NO: 2 or SEQ ID NO: 4. In some embodiments, the method of any preceding aspect comprises the nucleic acid encoding the RAG2 protein having at least 99% sequence identity to SEQ ID NO: 2 or SEQ ID NO: 4. In some embodiments, the method of any preceding aspect comprises the nucleic acid encoding the RAG2 protein having SEQ ID NO: 2 or SEQ ID NO: 4.

In some embodiments, the method of any preceding aspect comprises the RSS having a 12 base pair (bp) spacer or a 23 bp spacer. In some embodiments, the one or more RAG proteins bind to one RSS with a 12 bp spacer and binds to one RSS with a 23 bp spacer. In some embodiments, the method of any preceding aspect comprises a RSS having a nucleic acid sequence comprising SEQ ID NO: 6, SEQ ID NO: 7, SEQ ID NO: 8, SEQ ID NO: 9, SEQ ID NO: 10, SEQ ID NO: 11, or a combination thereof.

In some embodiments, the method of any preceding aspect comprises the non-vertebrate cell further having a nucleic acid encoding a High Mobility Group Box I (HMGB1) protein. In some embodiments, the HMGB1 protein allows for more efficient V(D)J recombination. In some embodiments, the method of any preceding aspect further comprises a nucleic acid comprising SEQ ID NO: 5.

The mutant RAG protein can lead to RAGI mutation immunodeficiency and/or RAG2 mutation immunodeficiency, wherein primary immune disorders caused by defects in the RAGI gene and/or the RAG2 gene leading to recurrent infections, inflammation, autoimmunity, low B cell and T cell counts, chronic diarrhea, and neurological defects. Non-limiting examples of disorders caused by RAGI mutations and/or RAG2 mutations include classic SCID (TB-NK+immunodeficiency), LeakySCID/Omenn Syndrome (OS), and Combined Immunodeficiency with granulomas/autoimmunity (CID-G/AI).

In some aspects, disclosed herein is a method of treating or preventing a disease in a subject in need thereof, the method comprising obtaining a non-vertebrate cell; introducing into the non-vertebrate cell a nucleic acid encoding one or more recombination activating gene (RAG) proteins selected from RAG1, RAG2, or variants thereof and a recombination signal sequence (RSS); culturing the non-vertebrate cell of any preceding step under cell culture conditions to allow V(D)J recombination activity, wherein the non-vertebrate cell expresses an antibody, or a fragment thereof following V(D)J recombination; extracting and/or isolating the antibody, the TCR, or a fragment thereof; and administering the antibody, or a fragment thereof to the subject.

In some embodiments, the method of any preceding aspect comprises the non-vertebrate cell being a yeast cell, wherein the yeast cell includes but is not limited to a Saccharomyces cerevisiae (S. cerevisiae) yeast cell. The non-vertebrate cells of any preceding aspect are cultured in conditions known in the art to promote stability, viability, and growth. A non-limiting example of culture conditions include incubating said non-vertebrate cells in warm (such as for example 30° C.), slightly acidic (pH 4.0-6.0), sugary environments with aeration using rich media like YPD (Yeast extract, Peptone, and Dextrose), wherein the incubation often includes shaking or rolling liquid cell cultures to ensure oxygen and sugar supplies are well distributed across the cells.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Generating Combinatorial Diversity Via Engineered V(D)J-Like Recombination in Saccharomyces cerevisiae

V(D)J recombination is a fundamental process in adaptive immunity, enabling the diversification of both immunoglobulins and T-cell receptors in jawed vertebrates. V(D)J recombination allows the semi-random recombination of gene fragments that encode a subsequence of a T-cell receptor or antibody protein, thereby generating vast combinatorial diversity in the immune receptor repertoire, which is essential to the immune system's ability to recognize diverse antigens. V(D)J recombination in B cells allows creation of a diverse antibody repertoire with both junctional and combinatorial diversity. It is driven by the recombination-activating genes RAGI and RAG2, which are thought to have evolved in jawed vertebrates from the domestication of a transposon roughly 500 Mya.

The yeast S. cerevisiae is frequently used for antibody-fragment isolation and engineering because of their ability to surface display genetically encoded antibody libraries. Furthermore, yeast are a well-recognized model eukaryotic organism that has proven useful for studying more complex eukaryotic cellular processes. Yeast cannot generate antibody combinatorial diversity in vivo, as they are unable to perform V(D)J recombination. Instead, the combinatorial diversity of yeast-display antibody libraries typically is generated inside a mammalian host's B cells (e.g., human, mouse, llama) in vivo, and then this diversity is harvested ex vivo and imported into yeast cells using traditional molecular cloning techniques. It is contemplated herein that a yeast strain could diversify antibody sequences in a manner similar to B cells could be a helpful tool both towards studying aspects of V(D)J recombination and towards generating combinatorial diversity in vivo. Yeast, being evolutionarily distant relatives of jawed vertebrates, lack any homologs to RAGI and RAG2 or their transposon prototypes, and as such, have no ability to perform V(D)J recombination.

To highlight one genetic locus on which V(D)J recombination is performed, in pro-B cells, a full-length heavy chain antibody variable domain is formed through sequential recombination of an array of V, D, and J gene segments, with a D and J subunit being brought together first, followed by fusion of a V gene to the DJ sequence, hence V(D)J recombination. Each recombination event requires precise RAG-mediated DNA cleavage at specific sites adjacent to V, D, or J gene segments, followed by DNA repair, resulting in excision of intervening genomic DNA regions. The DNA cleavage step requires two RAGI and two RAG2 proteins to form a heterotetramer complex that binds to recombination signal sequences (RSSs). RSSs contain largely conserved heptamer and nonamer DNA sequences critical to RAG complex recognition separated by 12-bp or 23-bp spacer regions which are less conserved but still able to influence recombination activity. The RAG complex follows the ‘12/23 Rule’, meaning it binds one RSS with a 12-bp spacer (12-RSS) and one with a 23-bp spacer (23-RSS) before cleaving DNA. The genomic arrays of V, D, and J gene segments are organized and include RSSs such that a combinatorially diverse repertoire of full-length immunoreceptors is formed by immune cells.

The high mobility group box 1 or 2 (HMGB1 or HMGB2) proteins assist with RSS binding and cutting by bending 23-RSS DNA, which can substantially increase the rate of RAG-mediated DNA cleavage and eventual recombination. DNA strands are cleaved in two steps. First, the RAG complex nicks DNA at the 5′ end of each RSS, adjacent to the heptamer, creating a 3′ hydroxyl group. Then, an interstrand transesterification reaction occurs where the hydroxyl group attacks the phosphate on the complementary DNA strand, causing a double strand break (DSB) precisely between the RSS heptamer and the DNA that will encode an immune receptor. This process ultimately results in two DNA ends with hairpins, called the ‘coding’ ends, and two DNA ends with blunt ends that contain the RSSs, called the ‘signal’ ends.

The hairpinned coding ends must then be opened and repaired via the nonhomologous end joining (NHEJ) pathway to allow formation of a ‘coding joint,’ e.g., the V to DJ genetic fusion that results in a functional immunoreceptor variable region. To form this functional coding joint (and repair the genome), the hairpins are opened by a complex consisting of Ku70, Ku80, DNA-PKcs, and Artemis, and then NHEJ-mediated DNA ligation follows, requiring the enzymes XLF, XRCC4, and DNL4. In B cells, coding joints often have junctional diversity between the two fused gene segments caused by the hairpin opening and by template-independent addition of nucleotides added by polymerases during NHEJ, such as TdT. In addition to coding joint formation, the blunt RSS-containing signal ends of DNA are similarly ligated together to form a ‘signal joint’ that (1) does not encode an immunoreceptor and (2) has been excised from the genome.

Two prior studies have investigated the possibility of a yeast strain that can perform V(D)J recombination, showing that expression of murine RAGI and RAG2 in S. cerevisiae allows recombination of DNA bearing RSSs to form signal joints or causes transposition events. This important work also confirmed that signal joint formation required yeast NHEJ protein machinery, such as YKU70, LIG4, and the MRX complex (MRE11, RAD50, and XRS2), showing that yeast can repair signal joints similarly to mammalian cells. However, the level of signal joint formation seen in these studies was extremely low (~1 signal joint or 4 transposition events per 10 million cells), requiring detection via a highly sensitive, nested PCR assay of bulk DNA. As such, no cells bearing a recombination event were able to be isolated, and a phenotypic test meant to detect DNA cleavage activity failed because it was below the assay's limit of detection. Lastly, these prior studies did not attempt to detect coding joints.

In this work, RAG-mediated coding joint formation is demonstrated in S. cerevisiae. In some instances, the yeast-based coding joint formation relies on homology between the coding ends, but the present disclosure also demonstrates that homology is not required for repair recombination. Both RSSs and functional RAGI and RAG2 proteins are verified to require for this recombination, and recombination activity is controlled in part by their ability to physically localize in the yeast nucleus over the cytoplasm or nucleolus. The initial recombination rate was increased by over 7000-fold by applying codon optimization, varying protein combinations and characterizing truncations, and improving localization. The lower limits of homology needed to allow our assisted recombination to occur were explored and powerful effects were seen when altering the DNA between RSSs. In a separate assay, signal joints were formed and isolated, exhibiting another hallmark of V(D)J recombination in yeast. Importantly, the yeast strains were shown to effectively quantify the recombination efficiency of mutant RAG1 genes associated with immunodeficiency. Finally, in two separate demonstrations using split fluorescent proteins and scFvs, the system was used to generate RAG-enabled protein diversity. In this manner, the present disclosure showed, for the first time in a non-vertebrate host cell, the ability to study RAG1-associated immune deficiency and to generate in vivo combinatorial protein diversity.

### Methods

Strains, media and general cloning techniques.

NEB 10-beta E. coli (New England Biolabs) were used for all molecular cloning purposes. Most S. cerevisiae strains developed in this work were modifications of BY4742 (Ura-, Leu-). For experiments involving scFv recombination, a display-competent strain, EBY100 (Trp-, Leu-), was instead modified. E. coli were cultured in 5 mL of LB broth (Fisher Bioreagents) at 37° C. overnight with agitation. LB was supplemented with 34 μg/mL chloramphenicol (Sigma Aldrich) or 100 μg/mL ampicillin (Sigma Aldrich) antibiotic for selection. For selection of plasmids in S. cerevisiae with a LEU2 marker, synthetic leucine dropout media was prepared with either glucose (dextrose, SD-Leu) or galactose (SG-Leu), comprised of 0.69 g/L complete supplement mixture minus leucine (CSM-LEU, Sunrise Science), 6.7 g/L yeast nitrogen base (YNB) with ammonium sulfate (BD), and 20 g/L of glucose (Fisher Scientific) or galactose sugar (Sigma-Aldrich). Synthetic tryptophan dropout media (e.g., SD-Trp) or synthetic complete media (e.g., SD) were also prepared by replacing the CSM-LEU with 0.74 g/L CSM-TRP (Sunrise Science) or 0.79 g/L of CSM (Sunrise Science), respectively. G418 selection is incompatible with ammonium sulfate. Therefore, to generate SD-Leu+G418 plates, YNB was replaced with 1.7 g/L of YNB without ammonium sulfate (BD) and 1 g/L of monosodium glutamate (TCI) and supplemented with 300 μg/mL of G418 sulfate (Gold Biotechnology). For 5-fluoroorotic acid (5-FOA) selection, 1 mg/mL of 5-FOA (Gold Biotechnology) was supplemented to the media.

Rich media was also prepared by combining 10 g/L yeast extract (Thermofisher), 20 g/L peptone (Thermofisher), and 20 g/L glucose (YPD) or 20 g/L galactose (YPG). For integration selection plates, YPD was supplemented with 100 μg/mL nourseothricin (Gold Biotechnology) for NATR gene selection or 200 μg/mL hygromycin B (Invitrogen) for HPH gene selection. For yeast display of antibody fragments, SD-Trp or SG-Trp media was further buffered to pH 6.25 by adding 5.4 g/L Na2HPO4 and 8.56 g/L NaH2PO4·H2O. For both yeast and E. coli, solid media plates were made with the addition of 20 g/L of agar (Fisher Scientific). For protein gels, 1x tris-glycine SDS buffer was prepared by diluting 10× tris-glycine SDS buffer (Thermofisher) with ultrapure water. For western blot, 10×TBST buffer (Thermofisher) was diluted to a 1x concentration and was used for all membrane washes. When blocking or staining the membrane with antibody, 5 grams (g) of nonfat dairy milk powder was added in a final volume of 100 mL of TBST.

DNA was amplified by polymerase chain reaction (PCR) using KOD Hot Start polymerase (Sigma-Aldrich). Gibson or Golden Gate plasmid assemblies were performed following previously established protocols. Many plasmids were made with Golden-Gate-compatible “parts” (i.e., promoters, genes, terminators, and backbones) that came from a plasmid collection previously generated for metabolic engineering applications. E. coli transformations were carried out using electrocompetent cells. All plasmids were sequence confirmed with whole-plasmid, nanopore sequencing (Genewiz). For yeast transformation of plasmids as well as linear DNA fragments for genomic integration, a high-efficiency, lithium acetate transformation method was followed.

### Strain Design and Cloning.

All RAG1, RAG2, and HMGB1 genes and their variants expressed in this work were integrated into the yeast genome, generating a variety of engineered strains. A summary of every strain developed in this work is provided in Table 1. In general, the strategy for all strain construction involved first making expression cassettes on integration-compatible plasmids. This was followed by co-transformation of PCR-amplified, linear fragments of DNA, including a selection cassette, that would integrate simultaneously at a targeted locus. Each linear fragment had ~50 bp of homology to the adjacent fragments. The two terminal fragments were amplified from genomic DNA and had ~400 bp of homology to the target locus as well as 50 bp of homology to the first and last linear fragment. Integration was directed toward sites which have been previously shown to be support robust heterologous gene expression. Following transformation, the presence of each integrated gene was confirmed through PCR of purified genomic DNA using a YeaStar Genomic DNA Kit (Zymo Research).

Two strains were created to easily visualize the nucleus and nucleolus during fluorescence microscopy. By tagging endogenous yeast proteins NAB2 and NOP56 with mCherry, it is possible to visualize the nucleus and nucleolus, respectively. To build these strains, NAB2 and NOP56 along with their native promoter were PCR amplified from genomic DNA prepared from BY4742. Then, using Gibson assembly, two plasmids were made which fused NAB2 and NOP56 to mCherry on the C-terminus of each protein, connected by a short peptide linker. After amplification, the mCherry-tagged genes were integrated at YPRCΔ15 with NAT selection in BY4742 cells.

To prepare recombination genes for integration, first yeast-codon-optimized, full-length RAG1 and RAG2 (human and mouse sequences for each), and human HMGB1 were synthesized as linear fragments by Twist Bioscience. Sequences of synthesized genes can be found in the sequence listing. Native murine RAGI and RAG2 sequences were isolated from CT26 murine colorectal immortalized cells using a mammalian genomic DNA prep kit (Quick-DNA Miniprep Kit, Zymo Research). Gene fragments were amplified and cloned into Golden-Gate-compatible vectors using Gibson cloning. In addition to the full-length RAG proteins, core versions of the proteins, RAG1core (amino acids 383-1006 of 1040 total) and RAG2core (amino acids 1-383 of 527 total), were also cloned. Identical truncations were made for the mouse wild-type genes, and a similar truncation was made for human RAGI (amino acids 387-1011). The strong, galactose-inducible promoters of GALI and GAL2 were selected for RAG1 and RAG2 expression, respectively, while a strong, constitutive, heterologous promoter was selected for HMGB1. To augment subcellular localization, a nuclear localization signal (NLS) comprising the first 28 residues from histone H3 (HHT1) was selected for addition to RAG1core and RAG2core. The NLS was amplified from genomic DNA and inserted at the 5′ end of both genes using Gibson assembly.

Integration-ready plasmids, which include promoters, terminators, and 60-bp flanks for integration were then cloned using Golden Gate. The full expression cassettes with flanking homology were then amplified from these plasmids along with a nourseothricin (NAT) selection cassette using PCR. These linear fragments were integrated simultaneously at the YPRCr3 locus of strain BY4742 and plated on YPD+NAT. Due to the modular design, all desired combinations of RAG1, RAG2, and HMGB1 were created similarly.

To generate eGFP-tagged RAG plasmids, an eGFP expression plasmid was first made with Golden Gate from the part collection. Then fragments were amplified from the RAG expression plasmids and a fragment from the eGFP plasmid and combined in a Gibson assembly such that eGFP would be fused to the C-terminus of each protein with a (GGSGG) 2 linker. All eGFP-tagged RAG constructs were amplified and integrated into NAB2-mCherry and NOP56-mCherry strains at the YORWA22 locus with LEU2 selection.

Additional modifications were made to the original RAG expression plasmids to generate the deleterious RAGI mutants, RAG2-T490A, and RAGI truncation variants. For RAG mutants, primers were ordered to introduce the desired mutation, and the expression cassette was amplified in two pieces which were split over the mutation site. In this way, a subsequent Golden Gate reaction could reassemble the original construct with the new mutation. For the RAGI truncation variants, DNA was amplified at the desired truncation position using the full-length RAGI as template. Promoters and terminators were then added using Golden Gate assembly. All these protein variants were integrated at YPRCT3 in BY4742 with NAT, similar to the standard constructs.

### Recombination Target Plasmid Design and Construction.

A full list of the plasmids used to assay recombination is provided in Table 2, and the full set of target sequences are provided. For antibiotic resistance recombination target substrates, the plasmids were generated using iterative rounds of Golden Gate cloning. G418R, an aminoglycoside O-phosphotransferase from Aeromonas veronii which confers resistance to G418 (also known as Geneticin), was selected as a target resistance marker. First, plasmids pY112-psmTEF1-G418R-Tefm1 and pY112-pFBA1-klURA3-tklURA3 were made using Golden Gate and the part plasmid collection mentioned above, where klURA3 is from the yeast Kluyveromyces lactis and smTEF1 is from Saccharomyces mikitae. Next, plasmid pY112-CJA-UP-H20 was designed using PCR of these plasmids along with a strong, heterologous promoter, pspTDH3, followed by Golden Gate assembly. RSSs were added in this step with primers. Variants of pY112-CJA-UP-H20 were generated similarly using Golden Gate assembly. Backbone pY112 contains a LEU2 cassette and was selected for in yeast using-LEU media.

A separate target plasmid was designed to detect signal joints. URA3 and eGFP expression cassettes were made using Golden Gate. Then RSSs were added using nested PCR, and the final plasmid, pY112-SJUE, was assembled via Golden Gate. Due to plasmid incompatibility with 5-FOA selection, the SJUE target was amplified and integrated at the NRT1/GYP1 locus in strain AC518 using the URA selection built into the fragment.

To make constructs that would allow RAG-mediated production of combinatorial diversity within fluorescent proteins, plasmid pY112-psmTEF1-GFP-tEFM1 was first generated with Golden Gate. Gene segments for Sapphire and Azurite were synthesized by Twist Bioscience, each with a flanking 23-RSS and terminator. To avoid expansive homology between fluorescent protein coding regions, the Sapphire(S) and Azurite (A) gene sequences were codon optimized to remove homology regions greater than 10 bp to each other and to eGFP (E). From these components, plasmids pY112-CJCG-XX-H20 (where XX=ES, SE, EA, and ESA) were assembled by Golden Gate of fragments generated using PCR with tailored primers. The region between the RSSs was taken from pY112-CJA-U-H20. Single color controls for eGFP, Sapphire, and Azurite were also generated that had intact genes and the smTEF1 promoter. These plasmids were made by DNA amplification using modified primers and the recombination plasmids as template followed and Golden Gate.

Constructs that would allow RAG-mediated production of combinatorial scFv diversity were designed in a similar manner to the split GFP plasmids. scFvs sequences were taken from a CDR3 library previously sorted against both PSCA and GPC3 followed by isolation. AGA2 and the first half of the scFv were followed by a 12-RSS and an intersignal region. Then a 23-RSS, second half of the PSCA scFv, and a terminator were followed by a second 23-RSS, the second half of the GPC3 scFv, and another terminator. The C-terminus of each scFv fragment is tagged with a unique peptide tag (Myc or FLAG), making it easier to discriminate which fragment recombined with the first half. The CDR3 which confers GPC3 targeting, along with a flanking 23-RSS and terminator, were synthesized by Twist Bioscience to remove homology to the anti-PSCA sequence. This plasmid, pY110-CJCS-PG, was transformed into a strain capable of both display and recombination. Plasmid pY110-CJCS-PG was created with Golden Gate using tailored primers. Backbone pY110 contains a TRP1 cassette and was selected for in yeast using—TRP media.

### GFP-Tagged Yeast Expression, Yeast Microscopy, and Image Analysis.

To quantify the relative expression of eGFP-tagged RAG1 and RAG2, cells were grown overnight in 0.5 mL of YPG media. 1×10⁶ cells were collected and rinsed with PBS. eGFP fluorescence was then measured using a BD FACSMelody. Data were analyzed and median fluorescence values gathered using FlowJo software. For confocal microscopy, yeast strains were grown overnight in 0.5 mL of YPG media. In the morning, yeast cultures were diluted to a concentration of 0.5×10⁷ cells/mL in 0.5 mL of fresh, warm YPG, and then cells were allowed to grow for an additional 6 hours. 1×10⁷ cells were collected, rinsed with phosphate buffered saline (PBS), then resuspended in 10 μL of PBS and transferred to a glass slide and protected with a No. 1.5 glass coverslip. Z-stack images with a spacing of 0.5 μm were collected using a Zeiss LSM 700 microscope with a 63x objective lens using oil immersion. Instrument gain was held constant for all measurements except for the eGFP channel of RAGI and RAG1core samples which have much dimmer eGFP signals.

Images were analyzed using Fiji/ImageJ to quantify the colocalization of the eGFP-tagged RAG proteins with mCherry-tagged NAB2 or NOP56. First, a maximum intensity z-projection was applied to the eGFP and mCherry channels for each image. A region of interest was created for each image by modifying the brightfield channel using the threshold and binary operations in ImageJ to create a mask for where cells were located. Then, the Pearson coefficient (no threshold) of each image was calculated using the Coloc 2 plugin in ImageJ. Each eGFP-tagged construct was imaged in biological triplicate, which were combined to calculate the mean and standard deviation.

### RAG2 Western Blot

To extract protein for western blot, cells were grown overnight in 10 mL YPG to a density 2.5×10⁷ cells/mL. At this point, 1 mL of culture was collected and spun down and the supernatant was removed. Next, the cells were resuspended in 800 μL of 0.1M NaOH and allowed to incubate at room temperature for five minutes. The cells were then spun down again, and the supernatant was completely removed. The pellets were resuspended in 60 μL of reducing Laemmli SDS sample buffer (Thermofisher) and incubated at 95° C. for five minutes. After one final spin down, the supernatant containing the soluble proteins was transferred to a new tube.

To perform the western blot, 6 μL of isolated protein solution were loaded per well into a 4-20% Novex tris-glycine gel (Thermofisher) and then run at 170V for 1 hour while submerged in TGS buffer. The proteins were transferred from the gel to a PVDF membrane using a Power Blotter Select Transfer Stack with the Power Blotter instrument. The membrane was washed and blocked with 40 mL TBST buffer containing 5% nonfat milk and gently mixed for 1 hour on a rocker at room temperature. Next, the membrane was stained with primary, polyclonal rabbit anti-RAG2 antibody (Thermofisher) at a 1:1000 dilution in 25 mL TBST with 5% nonfat milk for 1 hour. The membrane was washed with 1×TBST to remove excess primary antibody. The cells were then stained with a secondary, polyclonal, goat anti-rabbit-IgG antibody tagged with horse radish peroxidase (HRP, Thermofisher) at a 1:2500 dilution. The membrane was washed one final time with TBST. The HRP signal was then detected by adding 5 mL of Supersignal West Pico PLUS chemiluminescent substrate (Thermofisher). The membrane image was then recorded using a GE Amersham Imager 600. To compare the amount of protein loaded for each sample, another tris-glycine gel was prepared exactly as described above and then stained with Coomassie Brillant Blue (Thermofisher).

### G418 Resistance Recombination Assay

Prior to growing in liquid culture, cells were streaked on SD-Leu plates and allowed to grow for 2 days. Single colonies were picked from fresh plates and grown in 0.5 mL of SG-Leu in a round, 48-deepwell plate at 30° C. using a microplate shaker set to 700 rpm. After culture, the optical density (OD) was measured using a spectrophotometer from which cell concentration was calculated. Every two days, cultures were reinoculated into fresh SG-Leu media at a concentration of 0.2×10⁷ cells/mL. At indicated times points, yeast cells were plated on 6-cm diameter SD-Leu+G418 plates and spread with beads. To aid counting, the total amount of plated cells was varied based on the recombination efficiency of the strain. If the plated volume was less than 100 μL, sterile PBS was added before the culture such that the combined volume was 100 μL. Cells were allowed to grow for 2-3 days prior to manual colony counting. For sequence analysis of recombined colonies, colony PCR followed by Sanger sequencing (Genewiz) was carried out.

### 5-FOA Signal Joint Detection Assay

A slightly modified protocol to that of the G418 resistance test was followed for signal joint detection. YPG media was used in place of SG-Leu, and cells were passaged every day rather than every 2 days. After 8 days, cultures were plated on SD, 5-FOA plates, and cells were allowed to grow for 2 days.

### Combinatorial GFP Recombination Assay

Beginning with freshly plated single colonies, yeast were cultured in 0.5 mL of SG-Leu in a round, 48-deepwell plate at 30° C. using a microplate shaker set to 700 rpm. Every two days, yeast cultures were reinoculated into fresh SG-Leu media at a concentration of 0.2×10⁷ cells/mL. Cells were analyzed on a BD FACSMelody flow cytometer. Data were analyzed and recombination events were counted using FlowJo software with gates determined according to single color controls.

Combinatorial scFv Recombination Assay

Again, starting from freshly plated single colonies, yeast cells were cultured for 8 days in 0.5 mL of SG-Trp or SD-Trp, with reinoculation into fresh media at a concentration of 0.2×10⁷ cells/mL every two days. Yeast cultures were then reinoculated at a concentration of 0.5×10⁷ cells/mL in buffered SG-Trp and grown overnight to induce scFv surface display. To discriminate antigen binding to the two substrates, each sample was treated with two separate stains, one to detect PSCA binding and the other to detect GPC3 binding. 2×10⁶ cells were collected and rinsed with PBSF (PBS supplemented with 1 mg/mL bovine serum albumin), then stained with either biotinylated PSCA (ACROBiosystems) at 2 μg/mL or biotinylated GPC3 (ACROBiosystems) at 6 μg/mL for 60 minutes. After rinsing with PBSF, cells were stained with 10 μg/mL Streptavidin-PE (Thermofisher), and either 0.2 μg/mL anti-Myc-AlexaFluor647 (Cell Signaling Technologies, for cells that had been stained with PSCA) or 1.6 ug/mL anti-FLAG-APC (BioLegend, for cells that had been stained with GPC3). After 30 minutes, cells were rinsed again, and then resuspended in 400 μL PBSF. Fluorescence measurements were then collected for each sample using a BD FACSMelody. Due to the large difference between APC and AlexaFluor647 fluorescence intensity, different gain values were used for the PSCA/anti-Myc-stained cells versus the GPC3/anti-FLAG-stained cells. Data were analyzed and double positive events (with both antigen binding and peptide tag presentation) were counted using FlowJo software. Gates were determined using controls that display either the PSCA or GPC3 scFv.

### Statistics

All experiments were performed in biological triplicate (n=3), except where noted, and charts show the mean with error bars representing+one standard deviation. All statistical tests were performed with Graphpad Prism software.

### Results

### Expression and Localization of RAG1 and RAG2 in Yeast

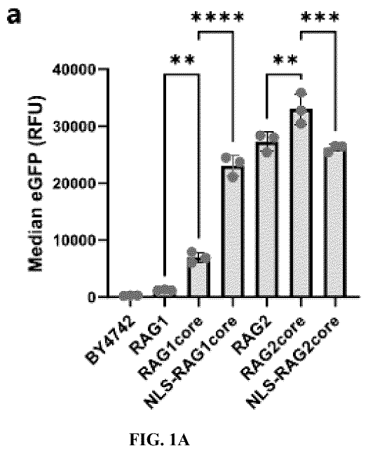

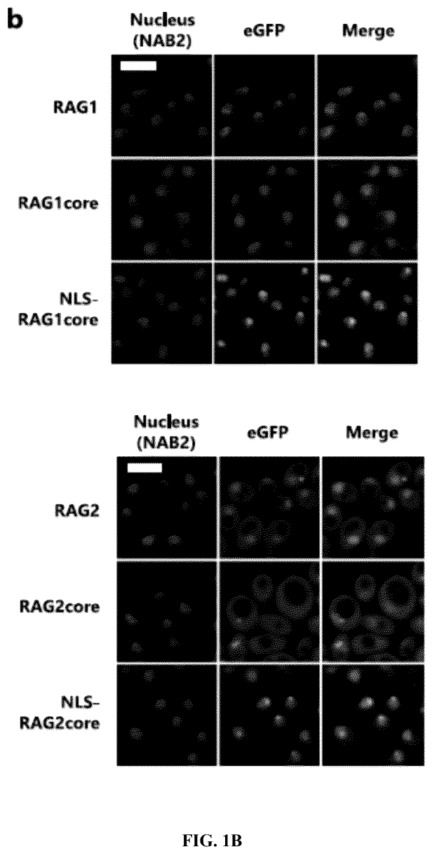

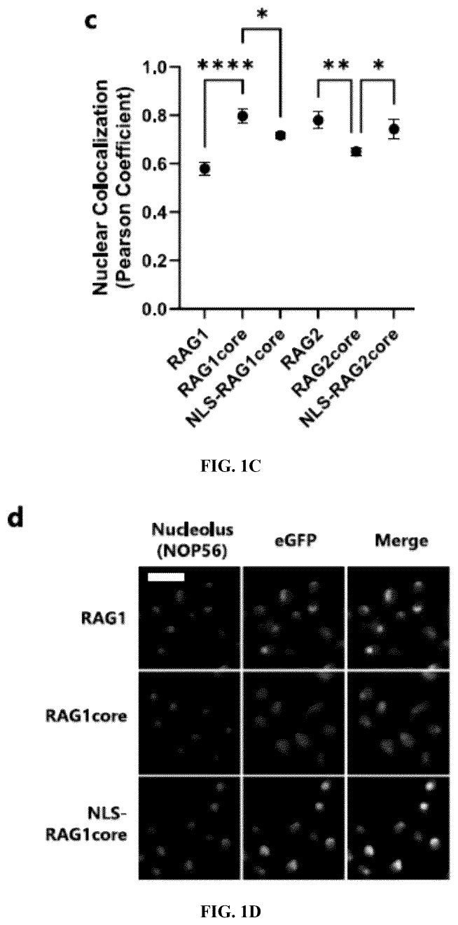

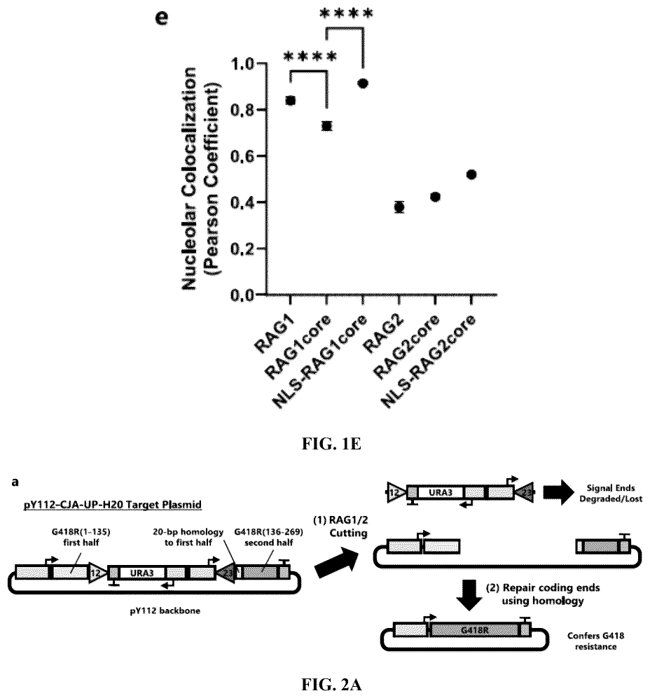

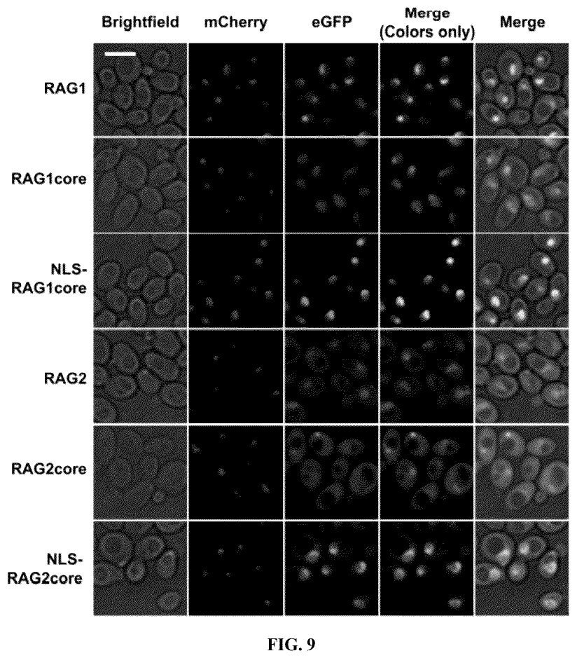

Based on the extremely low rate of signal joint formation mediated by RAGI and RAG2 co-expression in yeast previously reported, the relative expression and subcellular localization of full-length RAG proteins as well as their ‘core’ variants that have been truncated were first analyzed to allow for easier heterologous expression and purification. Subcellular localization is a critical regulator of RAG activity in mammalian cells, especially the localization of RAG1 to the nucleolus, which downregulates recombination activity in a pre-B cell model system. Murine RAG proteins and their core versions were C-terminally tagged with eGFP and expressed under control of strong, galactose-inducible promoters. eGFP-tagged proteins were integrated into yeast strains engineered to express either NAB2-mCherry or NOP56-mCherry fusion proteins, as they allow visualization of the nucleus and nucleolus, respectively. The RAG1core version had much higher expression than the full-length protein, as gauged by eGFP fluorescence intensity, whereas RAG2 expressed well as either a full-length protein or truncated to its core version (FIG. 1A). The full-length RAGI protein localized predominantly into the nucleolus, whereas RAG1core localized to the nucleus, consistent with work in mammalian cells (FIGS. 1B-1E and FIG. 8). The full-length RAG2 protein displayed the desired nuclear localization, while RAG2core was dispersed in the yeast cytoplasm (FIGS. 1B and 1C). Neither RAG2 nor RAG2core localized to the nucleolus (FIG. 1E and FIG. 9).

The present disclosure contemplates improving RAG1core and RAG2core nuclear localization by fusing a strong nuclear localization signal (NLS) from histone H3 (HHT1) to the N terminus of each enzyme. Surprisingly, expression was greatly improved for NLS-RAG1core though the localization shifted away from the nucleus and towards the nucleolus (FIGS. 1A-1E). In contrast, NLS-RAG2core appeared to decrease in expression relative to RAG2core, but the localization to the nucleus was improved (FIGS. 1A-1C). Based on the much higher expression and better nuclear localization of RAG1core and NLS-RAG1core relative to full-length RAGI, these variants are contemplated to outperform RAGI in recombination. For RAG2, based on the superior localization of full-length RAG2 and NLS-RAG2core compared to RAG2core, one of these variants are contemplated to be optimal for recombination.

### Enabling and Improving Homology-Assisted Coding Joint Formation in Yeast.

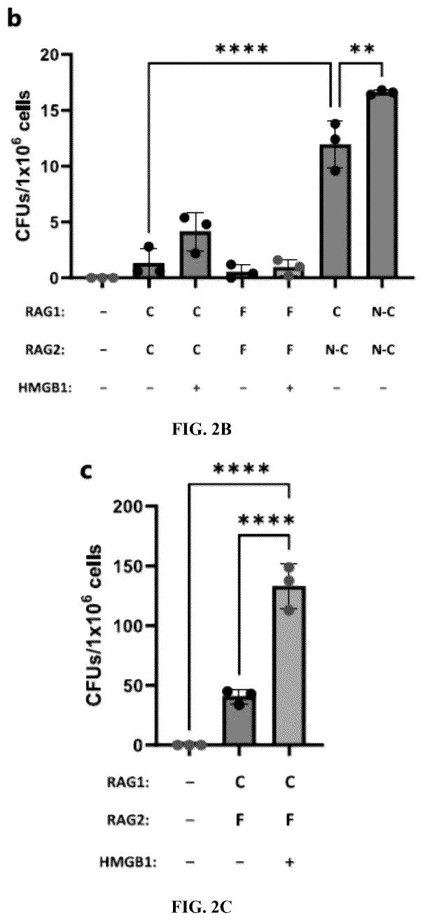

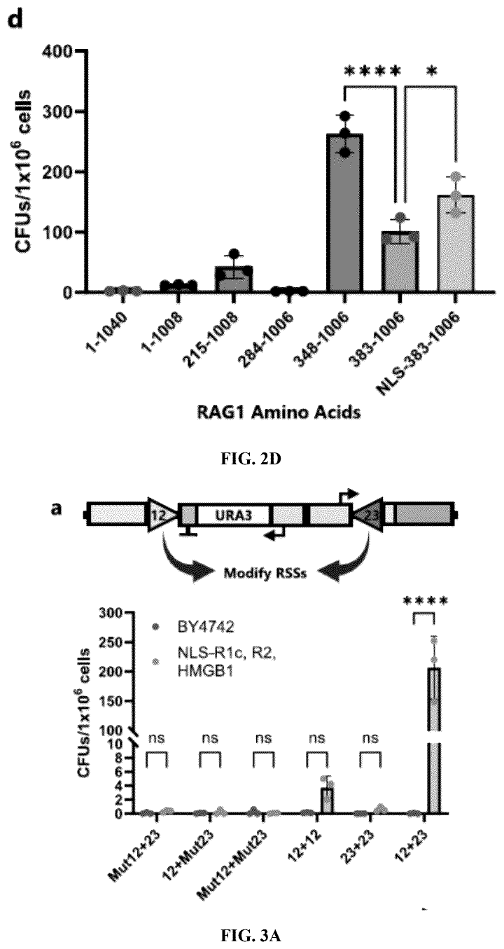

Given that S. cerevisiae prefer homology-directed repair (HDR) over NHEJ when resolving DSBs, a recombination substrate suited for repair inside a yeast host was sought. It was contemplated that adding a small region of homology (i.e., 20 bp) between two coding ends that would arise after RAG-mediated cleavage would allow the yeast to repair the DSB more effectively without greatly increasing RAG-independent recombination. Therefore, a G418 resistance (G418R) gene was split by inserting a 2-kb piece of DNA flanked by a canonical 12-RSS and 23-RSS. The 20 bp at the 3′ end of the first G418R fragment was duplicated at the 5′ end of the second fragment (FIG. 2A). The RSSs were oriented to have the heptamers next to the split G418R fragments, such that homology-assisted coding joint formation would reform an intact G418R resistance gene. The DNA sequence between the RSSs contained a URA3 expression cassette and a strong promoter pointed at the 23-RSS. The final recombination-substrate containing plasmid was dubbed pY112-CJA-UP-H20.

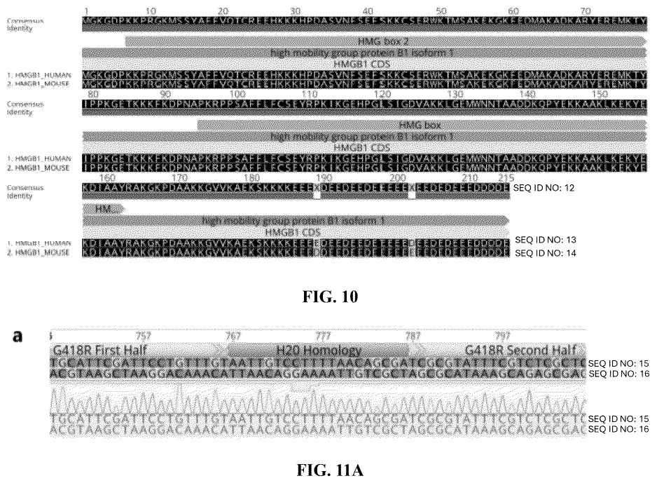

To begin to gauge the ability of RAG1/2 co-expression to mediate recombination in S. cerevisiae, yeast strains that expressed combinations of the murine RAGI, RAG2, and (optionally) human HMGB1 proteins were engineered. Full-length or core RAGI and RAG2 variants were placed under control of galactose inducible promoters, while HMGB1 was expressed constitutively if included. Human and murine HMGB1 sequences are nearly identical, with only two conservative amino acid substitutions differentiating the two (FIG. 10). For initial tests, all proteins were integrated into BY4742 strains that harbored the pY112-CJA-UP-H20 plasmid. Single colonies of each strain were picked, cultured, and spread on G418 antibiotic plates to detect RAG-assisted recombination.

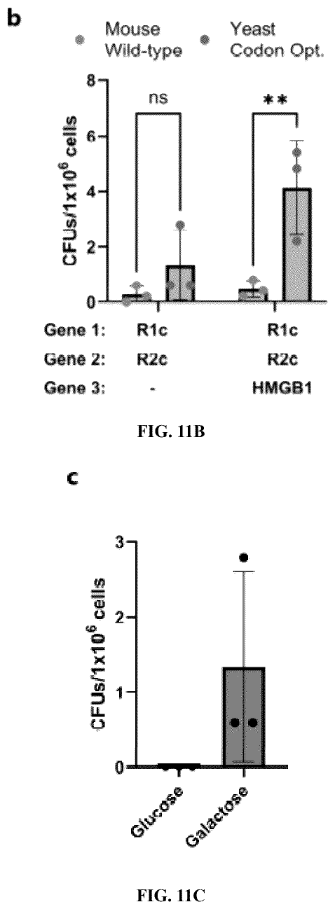

Murine RAG1core and RAG2core mediated roughly 2— to 4-fold more recombination than the full-length RAGI and RAG2 proteins, with or without co-expression of HMGB1 (FIG. 2B), and Sanger sequencing of nine colonies showed a seamless recombination of the G418R gene (FIG. 11A). Low recombination levels enabled by full-length RAG pairings are attributable to poor expression of full-length RAGI in yeast. In this vein, utilizing yeast-codon optimized RAG1core and RAG2core sequences improved recombination by over 5-fold compared to wild-type murine DNA sequences, once again with or without HMGB1 (FIG. 11B). In addition, culturing yeast in glucose media, such that RAG1core and RAG2core expression were strongly repressed, yielded no G418-resistant colonies (FIG. 11C).

Improvement in recombination rates mediated by co-expressing HMGB1 occurred in both full-length and core RAG contexts and were roughly similar (2— to 3-fold, FIG. 2B), which is supported by the context-independent mechanism of action for HMGB1-bending 23-RSS DNA to assist RAG-cleavage. Therefore, HMGB1 was typically co-expressed in the following studies geared towards improving recombination levels. We next wanted to determine if the higher nuclear localization of NLS-RAG2core or full-length RAG2 impacted recombination rates compared to the cytoplasmic localized RAG2core. NLS-RAG2core led to an over 8-fold improvement in recombination compared to unaltered RAG2core, as expected based on its improvement in nuclear localization (FIG. 2B). Even better, pairing the full-length RAG2 with RAG1core led to a 30-fold improvement relative to a strain with RAG2core (FIG. 2C).

Varying RAG1 length further improves coding joint formation.

Based on the notable improvement full-length RAG2 provided compared to RAG2core, it was contemplated that alternate truncations of RAGI might also improve recombination. The RAG1core protein, spanning amino acids 383-1006, was designed to help purify catalytically active RAGI for in vitro experiments. However, multiple studies have shown that the removed regions of RAGI are important for recombinase regulation or rate. In particular, the RAG1 protein region between amino acids 348 and 383 contains a C2H2 zinc finger, while the region between 284 and 348 contains a zinc C3HC4 RING finger, which together comprise a dimerization domain. In addition, the region between 215 and 284 contains multiple stretches of basic amino acids and is important for RAGI localization and activity. Therefore, it was tested if utilizing larger RAGI variants affected recombination rates in our split G418R assay, by co-expressing RAG1 variants consisting of amino acids 348-1006, 284-1006, 215-1008, and 1-1008 with RAG2 and HMGB1, comparing to the RAG1core (383-1006) and full-length (1-1040) variants. Notably, RAG1 (348-1006) had a 2.5-fold improvement relative to the RAG1core construct (FIG. 2D). In stark contrast, RAG1 (284-1006) had very low activity, showing a complex relationship between inclusion of distinct RAGI domains and recombination activity in yeast. Lastly, adding an NLS to RAG1core provided a modest but clear benefit to recombination relative to RAG1core (FIG. 2D). Given its performance, the strain containing NLS-RAG1core , full-length RAG2, and HMGB1, and labeled AC518, was used for many characterization studies described hereafter.

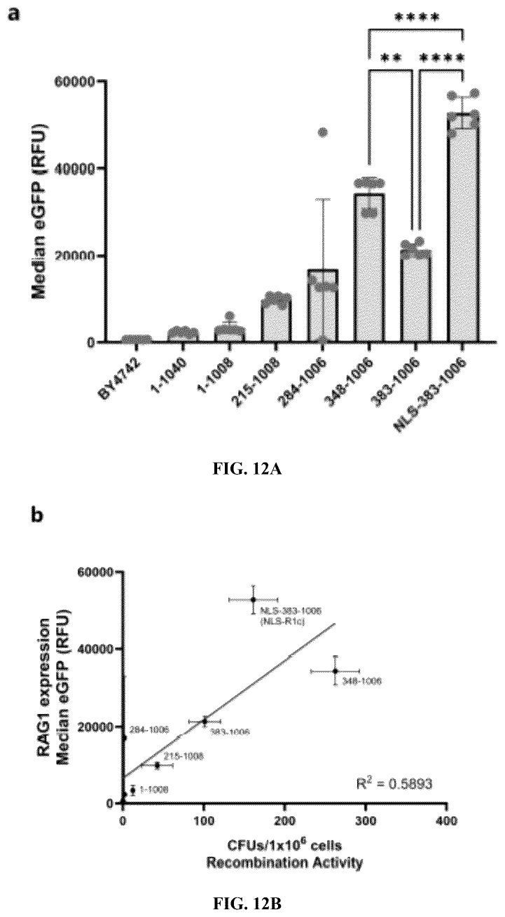

To elucidate the effect RAGI truncation has one expression, each truncation was tagged with eGFP and integrated into yeast. Using flow cytometry, fluorescence was measured for each construct (FIG. 12A). In general, higher expression correlated with higher recombination activity (FIG. 12B), but the correlation was not particularly strong, showing other factors such as localization or DNA binding capability also impact recombination activity.

### RAG1 and RAG2 Mutant Characterization; Additional Protein Factors Influencing Recombination

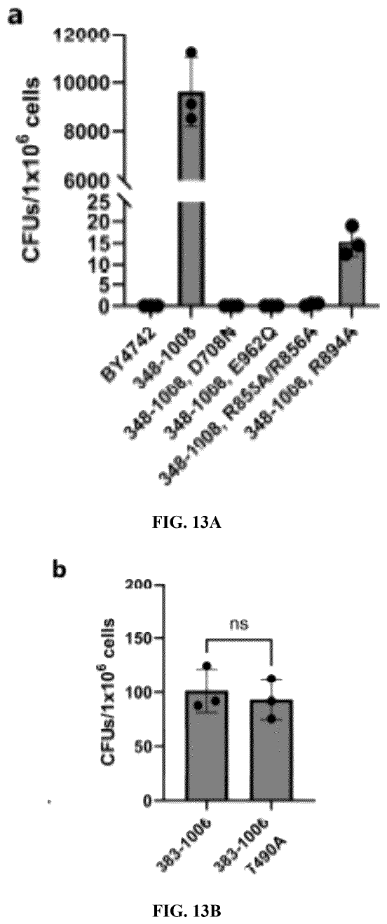

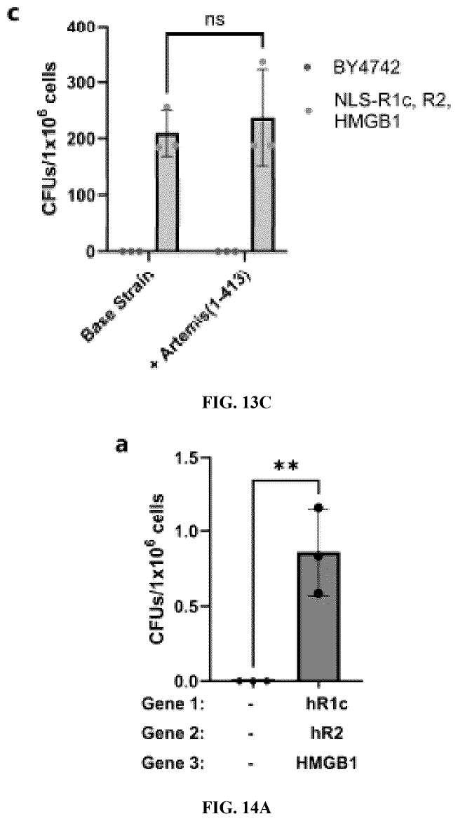

To confirm that the homology-assisted recombination was reliant on the catalytic activity of RAG1, two strains were constructed harboring RAGI mutants with essential catalytic residues mutated, either D708N and E962Q. While these catalytically dead mutants have been shown to have the ability to bind RSS sequences, recombination was completely abolished in these yeast strains, indicating RAGI catalytic activity is required for homology-assisted V(D)J recombination (FIG. 13A). Next, it was tested if RAGI nicking (without double-strand cleavage) was sufficient to stimulate recombination. RAGI mutants R855A/R856A and R894A have been shown to be able to nick DNA adjacent to RSS sequences but lack the ability to cleave DNA via hairpin formation. Two strains were created that used these mutants, and RAGI recombination was reduced over 500-fold for each strain (FIG. 13A). Therefore, yeast homology-assisted recombination strongly benefits from a RAGI that can efficiently cleave double-stranded DNA, similar to what is seen in mammalian cells.

The present disclosure also demonstrates employing engineered RAG2 variants that could enhance recombination. In particular, it has been shown in mammalian B cells that, in a cell-cycle dependent manner, RAG2 is phosphorylated at a threonine at residue 490 (T490) by CDK2 and degraded, limiting RAG2 activity to the G1 phase. Yeast have a CDK2 homolog, CDC28, which may affect RAG2 in a similar manner. Mutation of T490 to an alanine has been shown to abolish this cell-cycle dependent degradation. When recombination rates mediated by full-length wild-type RAG2 and a RAG2-T490A variant were compared, RAG2-T490A did not mediate a significant improvement (FIG. 13B).

One bottleneck for coding joint repair post-cleavage is the opening of coding end hairpins. In B cells, DNA-PKcs-phosphorylated-Artemis opens hairpins but a truncated Artemis (residues 1-413 of 692 total) has been shown to be constitutively active. Yeast-codon optimized Artemis (1-413) was integrated into strain AC518 and tested the rate of recombination. Artemis (1-413) did not significantly increase the rate of recombination (FIG. 13C).

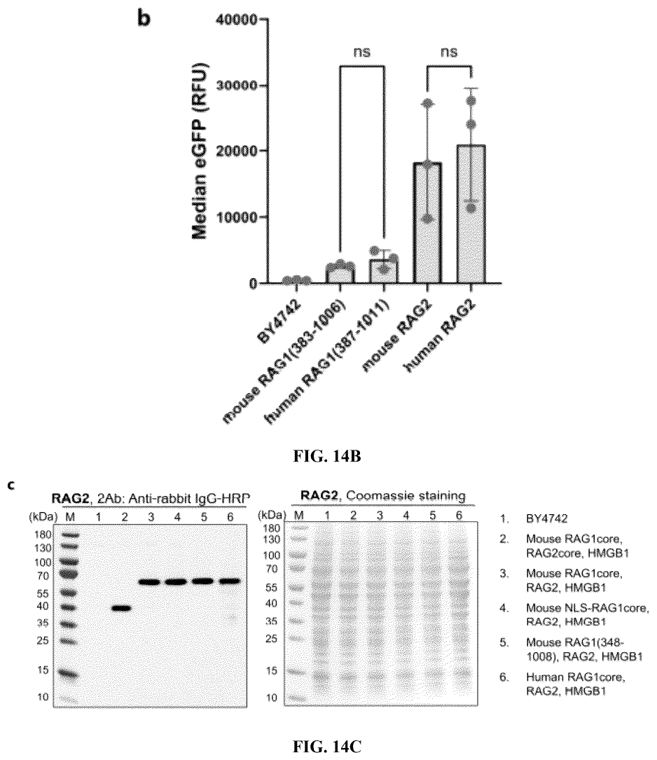

Encouraged by the improved recombination rates seen utilizing the murine RAG1core and full-length RAG2 proteins, the ability of the human RAG proteins to function in yeast was also tested, which has not been determined previously. A yeast strain expressing the human RAG1core , full-length RAG2, and HMGB1 had much lower recombination rates, <1% compared to its murine counterpart (FIG. 14A). While human RAG1core and RAG2core can be purified just like the more commonly used mouse proteins, the human proteins were considered to suffer from poor expression in yeast. As with the mouse RAGI truncations, the expression of human RAG1core and RAG2 by fusing each to eGFP was checked. Surprisingly, there was no significant difference in fluorescence between mouse and human RAG1core and RAG2 (FIG. 14B). To further validate the eGFP tag data, a western blot was performed to directly measure the amount of RAG2 produced by the cells. Human and mouse RAG2 were easily detected at comparable levels (FIG. 14C). Therefore, the data indicate that the poor performance of human genes relative to mouse is not caused by deficient expression. While this was the first time that human RAG sequences mediated recombination in yeast, the study proceeded with the better performing murine RAG proteins for further characterization and application. Adding an NLS to RAG1core appeared to improve recombination (FIG. 2B), so this RAGI core-NLS was combined with HMGB1 and full-length RAG2. Indeed, NLS-RAG1core provided a modest but clear benefit to recombination (FIG. 2D). Given its performance, this strain, dubbed AC518 (NLS-RAG1core , full-length RAG2, and HMGB1), was used for many characterization studies described hereafter.

### Non-Protein Factors Influencing Coding Joint Recombination Activity in Yeast.

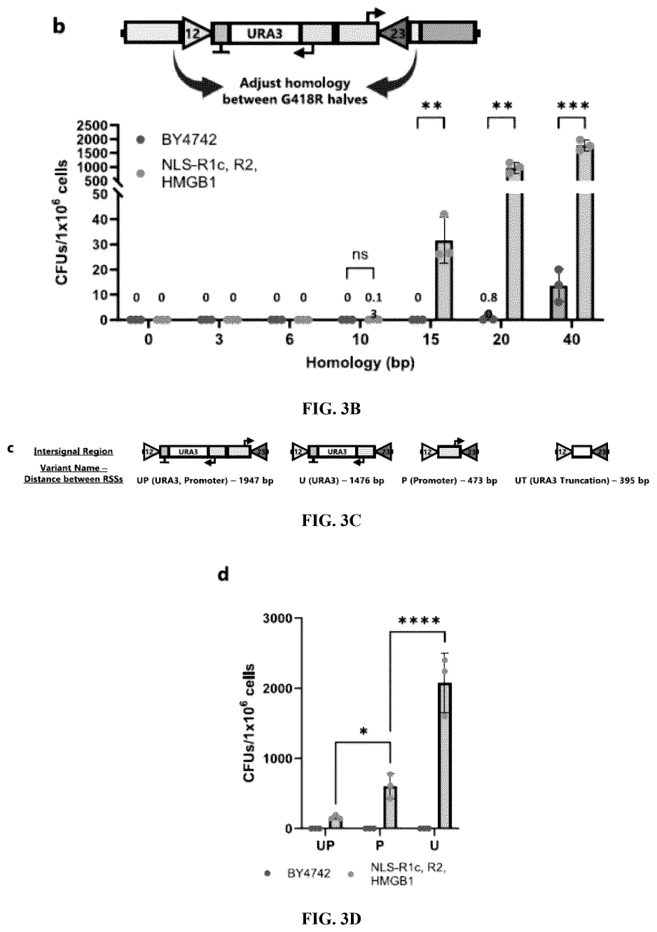

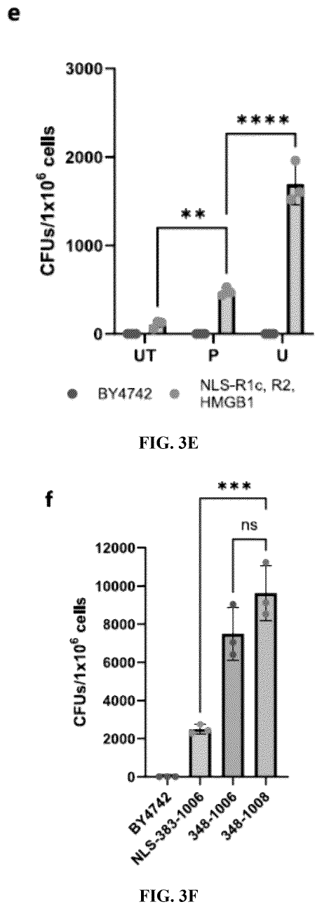

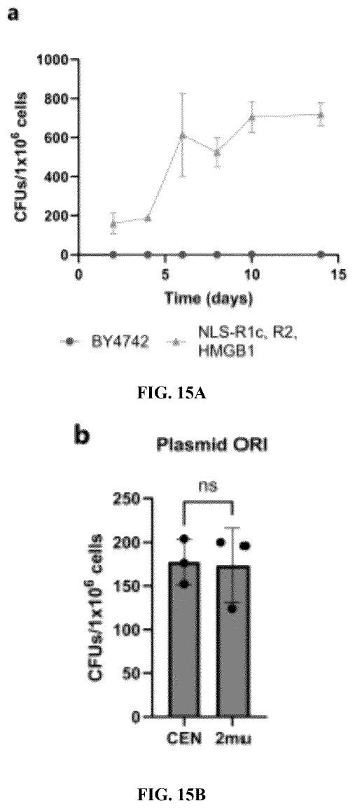

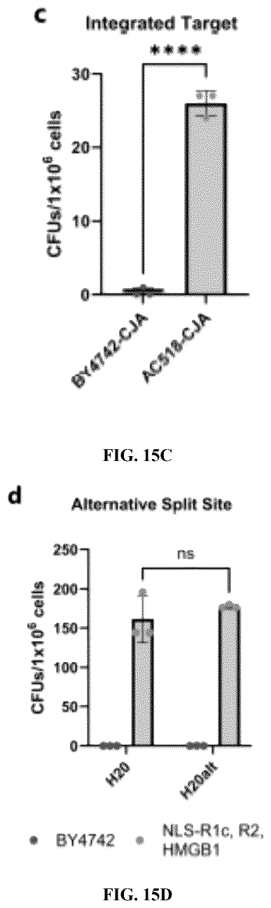

To verify that the mechanism of recombination in yeast required both RSSs, variations of pY112-CJA-UP-H20 detection plasmid that had mutations within the sequences of the 12-RSS, the 23-RSS, or both the 12- and 23-RSSs were generated (Table 3). Yeast strains harboring plasmids with mutated RSSs yielded no colonies, showing that correct 12- and 23-RSSs are required and indicating that the traditional mechanism for RAG cleavage, including the 12/23 Rule, was being followed in yeast (FIG. 3A). Next, in the split G418R recombination assay, both RSSs are eliminated from the plasmid, such that recombination to form the coding joint-bearing plasmid is irreversible. Therefore, the study sought if the percentage of yeast harboring a recombined plasmid, and hence a G418R gene, in a culture would increase over time. A time-coursed analysis of recombination levels was performed in yeast cultures expressing RAG1core , full-length RAG2, and HMBG1, plating cells every two days for fourteen days total (FIG. 15A). The percentage of yeast harboring recombined coding-joint plasmids increased over time, with a fivefold increase at fourteen days relative to four days.

In yeast, 20 bp of homology is approaching the lower-limit needed to allow homology-mediated DNA repair, and was therefore chosen as the level of overlap in homology-assisted coding joint formation plasmid, pY112-CJA-UP-H20. It was further contemplated if RAG1/2 expressing yeast strains could still mediate recombination if the level of overlap was reduced, therefore the impact of utilizing plasmids with 0, 3, 6, 10, 15, 20, or 40 bp of homology between the two G418R fragments was tested (FIG. 3B). The rate of recombination decreased drastically with decreasing overlap length, such that strains harboring plasmids with 10 bp of overlap had an extremely low level of recombination, while those with less had no detectable recombination. Sanger sequencing of four colonies confirmed that the repair was still accomplished without error with 10 bp of homology. In addition, wild-type yeast cells harboring split G418R constructs with 40 bp of homology began to have detectable but still low recombination, showing that utilization of the 20 bp overlap was optimal to detect recombination events driven by RAG1/2 activity.

To further increase the recombination rate, we reasoned that a high-copy, 2u plasmid would increase the likelihood of G418R formation relative to the pY112-CJA-UP-H20 construct which uses a low-copy, CEN/ARS origin of replication. After creating a 2u plasmid that is otherwise identical to pY112-CJA-UP-H20, however, the rate of colony formation was unchanged (FIG. 15B), showing that plasmid origin of replication does not strongly impact recombination. To further characterize the impact of the genetic context, the coding joint target was integrated into the yeast genome. Recombination events were still easily detected, but the rate was much lower than what was seen for the original plasmid (FIG. 15C).

To continue to gauge the importance of the recombination substrate plasmid design, additional alterations were made to test the importance of the split site within the G418R gene as well as the length and composition of the intervening DNA between the 12- and 23-RSS. Splitting the G418R gene in a different location (pY112-CJA-UP-H20alt) did not substantially alter recombination rate, showing that the initial G418R split site was not exceptional (FIG. 15D). Next, substrate plasmids were created in which (a) the Ura3 selection cassette was removed but the promoter pointing towards the 23-RSS was left, decreasing the distance between the RSSs from ~2 kb to ~0.5 kb (i.e., “P”), (b) the strong constitutive promoter was removed but the Ura3 cassette was left, decreasing the distance between the RSSs to ~1.5 kb, (“U”), or (c) the promoter was removed and the URA3 cassette was reduced to a nonfunctional truncation of the URA3 gene only ~0.4 kb in length (“UT”, FIG. 3C). Removing only the larger URA3 cassette increased recombination activity by nearly 4-fold, while surprisingly, removing only the promoter led to an over 10-fold improvement in recombination (FIG. 3D). In contrast, the “UT” construct had the lowest activity of the three shortened intersignal sequences (FIG. 3E). These results highlight the sensitivity that RAG proteins have to the interspace region between the RSSs. They show that multiple factors, such as intersignal distance, transcriptional activity, or possibly the nucleotides adjacent to the RSS can combine to impact recombination efficiency.

The best target plasmid was tested with the highest-performing recombination strain characterized earlier, which uses RAGI (348-1006), RAG2, and HMGB1. RAG1core commonly ends at residue 1008, therefore a similar strain was also built which includes the additional two amino acids on the C-terminus (348-1008). Recombination rates reached nearly 10,000 CFUs/1×10⁶ cells—or 1% recombination-after four days (FIG. 3F). A significant difference between RAG1 (348-1006) and RAG1 (348-1008) was not observed. The nearly 1% rate of RAG recombination is beginning to approach the rates seen using transfection in non-lymphoid, mammalian cell lines. The rate was high enough to analyze events via flow cytometry and showed that the yeast-based platform disclosed herein is a viable alternative to mammalian cells for certain approaches, such as studying defects in RAGI and RAG2 proteins.

RAG-expressing yeast strains can also undergo signal-joint creating recombination events.

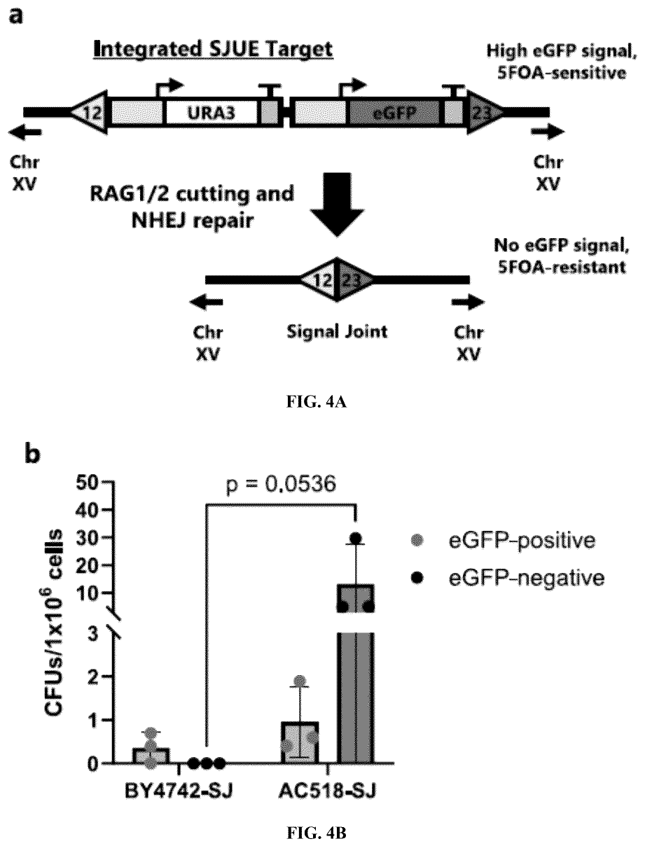

To further demonstrate that yeast recombination, as described in the present disclosure, follows a mechanism similar to what takes place in B and T cells, signal joints were examined. Signal joints are hallmarks of V(D)J recombination and are commonly used to assay recombination efficiency. Stringent phenotypic assays are challenging to design for detecting signal joints because they are formed through the ligation of the two RSSs, making it impossible to recombine a split resistance marker as presently done for coding joint detection. To overcome this challenge, a new construct, named SJUE, was designed having flanked URA3 and eGFP expression cassettes with RSSs such that signal joint formation would remove both markers from the construct (FIG. 4A). Cells harboring a successful recombination event would be resistant to 5-fluoroorotic acid (5-FOA) supplementation and eGFP-negative. eGFP was included within the excised region to allow discrimination between cells that had excised URA3 during signal joint formation and those with point mutations in the URA3 gene, which can occur naturally. To ensure a consistent, single construct copy number, SJUE was integrated into the genome of strains BY4742 and AC518.

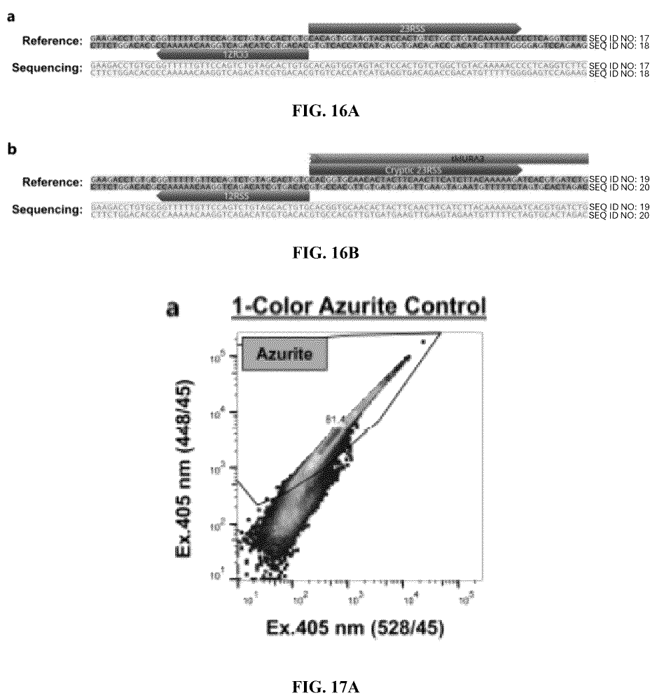

After an 8-day induction of RAG expression for each strain and then plating on 5-FOA plates, strain AC518 produced substantially more colonies than BY4742 (FIG. 4B). Importantly, for strain AC518, the vast majority of colonies were eGFP-negative, and while a BY4742 formed a small number of colonies, all were still fluorescent, indicating a signal joint had not formed. In previous efforts to detect signal joints in yeast, they were created on high-copy plasmids and were detected via a nested PCR assay, so it is difficult to compare the rate of signal joint formation to rates of prior studies. Importantly, the present work does mark the first time signal joint recombination events were isolated in a living yeast cell, and using Sanger sequencing, the recombination sites were directly analyzed from 15 colonies of ‘signal joint positive’ AC518. All were blunt ligations of the RSSs without error (FIG. 16A). Three colonies of AC518 were also sequenced that had maintained fluorescence, one of which had a URA3 point mutation. Surprisingly, the other two colonies harbored signal joints that appeared to have formed using the 12-RSS and a cryptic 23-RSS located in the URA3 terminator (FIG. 16B). Signal joint formation was not detected in BY4742 cells. The results appear to differ from previous work which showed a majority of recombination events using plasmid and integrated substrates underwent transposition. However, unlike the design disclosed herein, the signal joints from the studies conducted by others were not preserved in the genome after recombination, which may have biased their response toward transposition.

### Yeast-Based Analysis of RAGI Mutations Deleterious Toward V(D)J Recombination Activity.

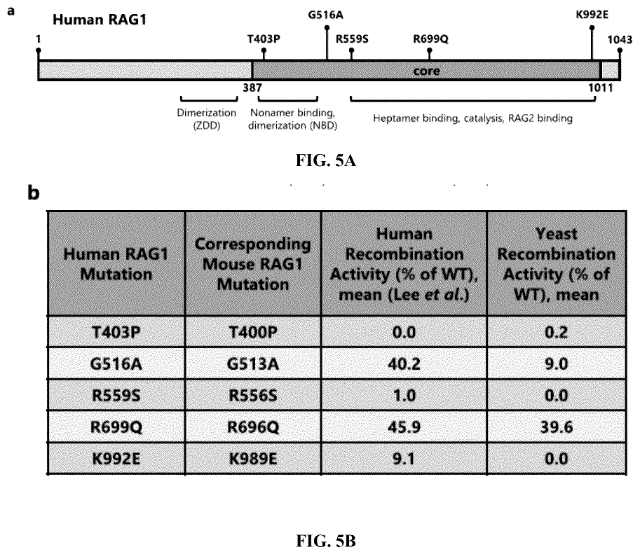

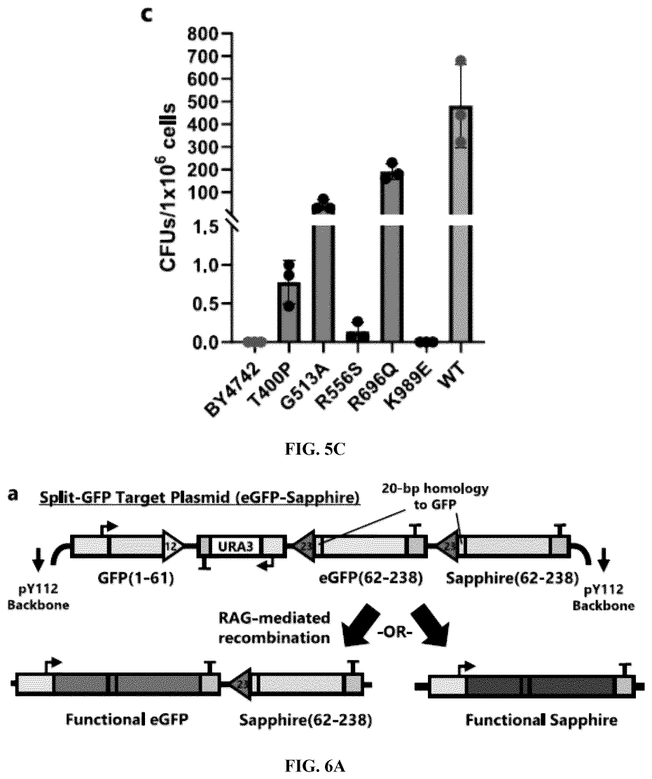

In mammals, deleterious RAGI mutations have been shown to cause varying degrees of immunodeficiency, including T⁻B⁻ severe combined immunodeficiency (SCID) and Omenn Syndrome. As many of these mutations directly affect the ability of RAGI to bind or cut DNA, it was contemplated that recombination rates seen in the present assay would inversely correlate with the severity of RAG1-mutation-associate immunodeficiency. To test this, five human RAG1 mutations known to have a range of outcome severity clinically were selected: T403P, G516A, R559S, R699Q, and K992E (FIGS. 5A and 5C). A mutant allele analysis was performed by introducing each mutation into their matching position in murine RAG1core (T400P, G513A, R556S, R696Q, and K989E) in the AC518 strain, and then testing for altered recombination ability (FIG. 5B). The replaced murine RAGI amino acid was identical to the initial amino acid found in human RAGI for each allele mutant, which is expected due to the >96% identity of the core proteins. The higher-activity pY112-CJA-U-H20 target plasmid was then used to assay for RAG-mediated recombination activity. The T400P and R556S mutations essentially eliminated recombination (FIG. 5C). G513A and R696Q RAG1core mutant activity also correlated with their activity in mammalian cells, retaining 10% and 40% activity respectively relative to a strain with an unaltered RAG1core . No activity from K989E RAG1core was detected. Still, coding joint formation in yeast largely correlates with RAG1 variant recombination in mammalian cells, demonstrating that yeast strains disclosed herein are useful in predicting pathogenic RAGI mutant activity.

### Enabling In Vitro, RAG-Generated Combinatorial Diversity of Fluorescent Yeast.

It was reasoned that the engineered yeast could generate in vivo combinatorial diversity, an essential process towards immunoreceptor repertoire generation in humans. The ability to generate in vivo combinatorial diversity, has not been demonstrated in an engineered model organism, but it could be a very useful tool for protein engineering. Combinatorial diversity in V(D)J recombination arises because one of multiple V, D, or J gene segments (each with flanking RSSs) is randomly selected for recombination, such that the number of combinations is the product of how many possibilities exist for each gene segment.

Two model protein classes were selected, fluorescent proteins and antibody fragments, to demonstrate and study the ability to create in vivo combinatorial diversity in the yeast-recombination strains. Constructs were first designed to generate fluorescent protein combinatorial diversity, selecting three variants of Aequorea victoria GFP: eGFP (F64L, S65T), Sapphire (S72A, Y145F, T2031), and Azurite (F64L, Y66H, Y145F, V150I, V224R).

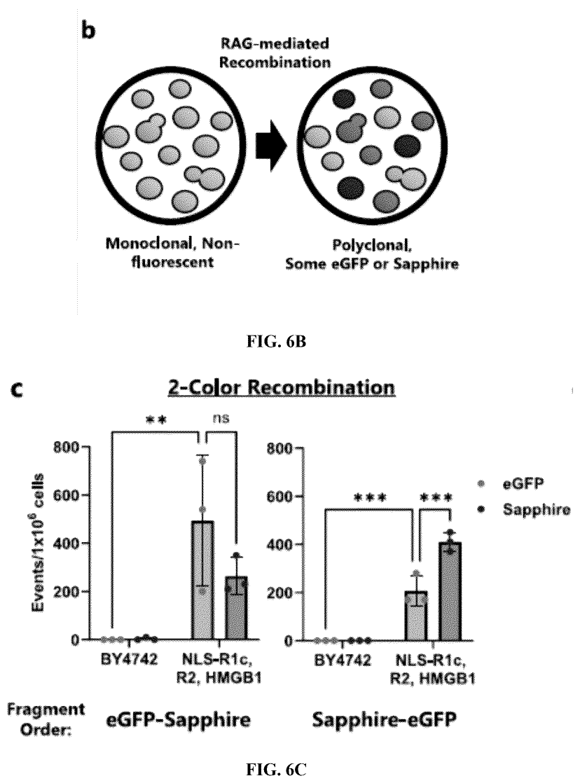

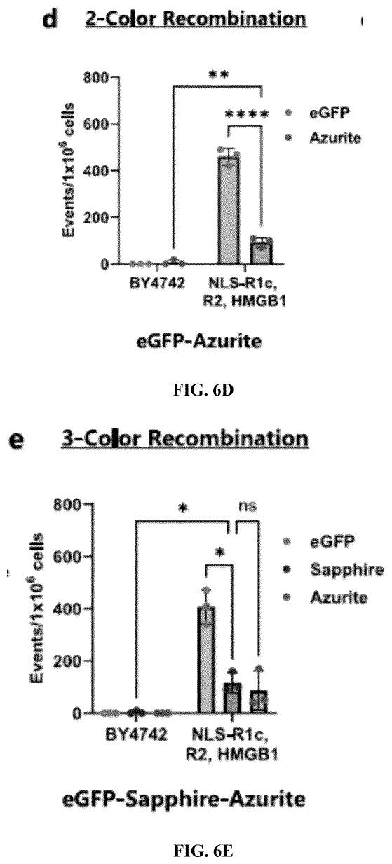

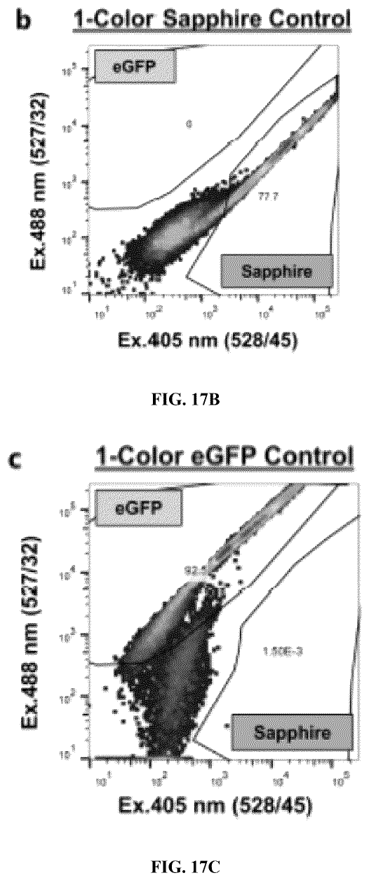

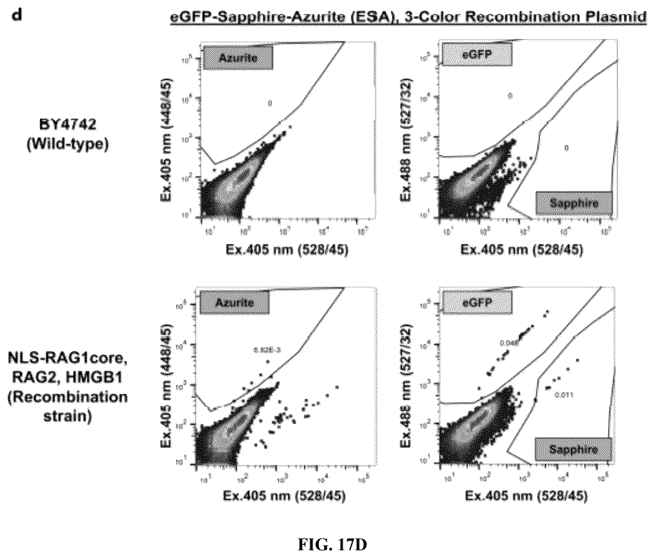

The combinatorial diversity substrates contained the first 62 amino acids of GFP, which are shared between eGFP, Sapphire, and Azurite, flanked by a 12-RSS, followed by DNA intersignal sequences and either two or three of the remaining gene sequence fragments of eGFP, Sapphire, or Azurite, each flanked by a unique 23-RSS (FIG. 6A). These 3′ sequences had different codon usages to prevent native, homology-directed recombination. It was reasoned that recombination events would be irreversible, as the 12-RSS is eliminated from the substrate plasmids following an event. In this way, a starting population of non-fluorescent cells induced to undergo homology-assisted, RAG-mediated recombination would create up to three new fluorescent subpopulations yeast cells, distinguishable via using flow cytometry (FIGS. 17A-17C).

Using a two-output combinatorial diversity plasmid substrate, in which a 23-RSS+eGFP 3′ fragment was followed by a 23-RSS+Sapphire 3′ fragment, recombination produced subpopulations of either fluorescent color, starting from a single non-fluorescent bulk population (FIGS. 6B and 6C). A higher prevalence of yeast cells expressing eGFP was observed, the fragment closer in proximity to the 12-RSS. When the order of the eGFP and Sapphire fragments were switched, a higher prevalence of Sapphire expressing cells was observed, showing that shorter DNA sequences between 12- and 23-RSS enhanced recombination likelihood (FIG. 6C). A separate target plasmid with eGFP and Azurite fragments showed similar behavior to the initial eGFP-Sapphire construct (FIG. 6D). When utilizing a plasmid substrate in which yeast-based recombination could create sequences for all three colors, eGFP, Sapphire, and Azurite subpopulations were detected after induction of RAG expression (FIG. 6E and FIG. 17D). Again, the prevalence of each color's subpopulations decreased with increasing distance of the 23-RSS from the 12-RSS (prevalence eGFP>Sapphire>Azurite). In all these studies, yeast strains lacking RAG expression did not create any fluorescent populations. Thus, it is shown that RAG-mediated recombination of a single starting population can generate functional in vivo combinatorial DNA diversity and can be extended to multiple substrates.

Extending In Vivo Combinatorial Diversity Generation to Displayed scFvs.

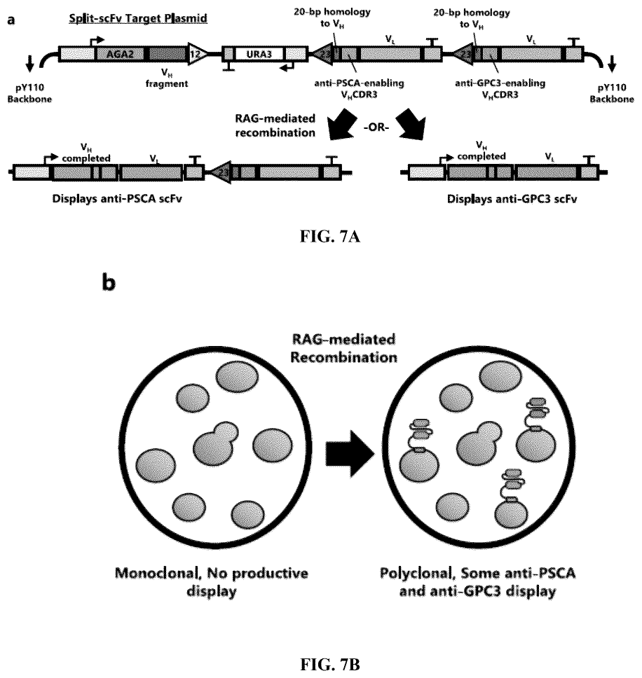

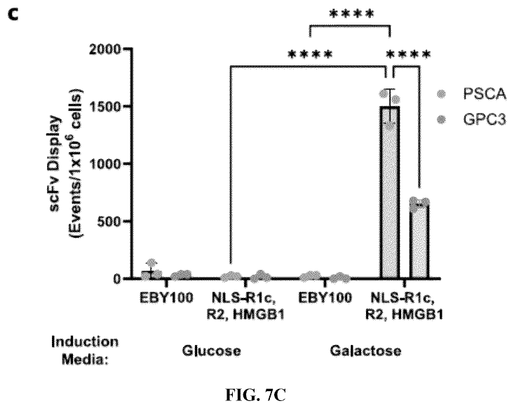

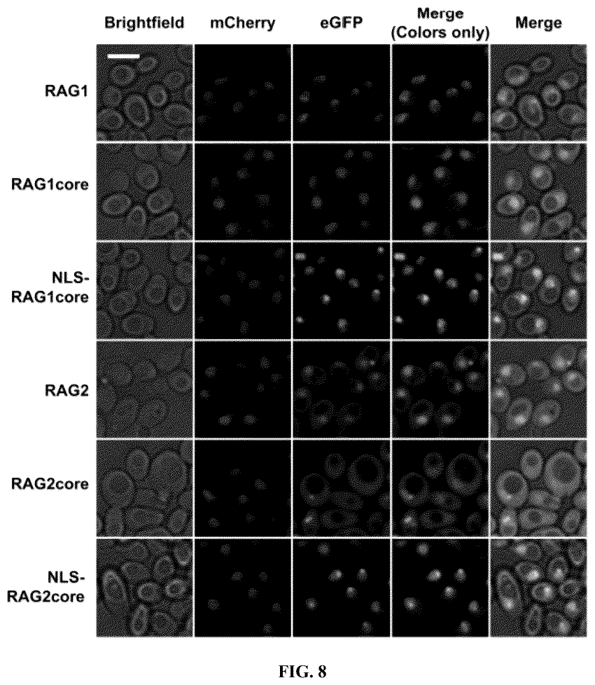

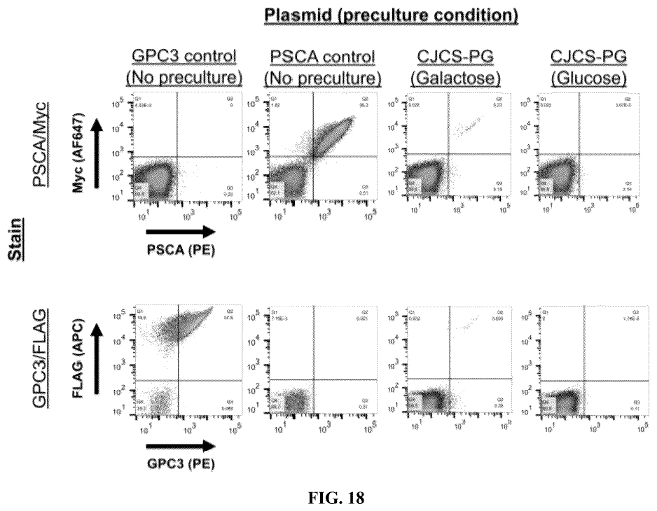

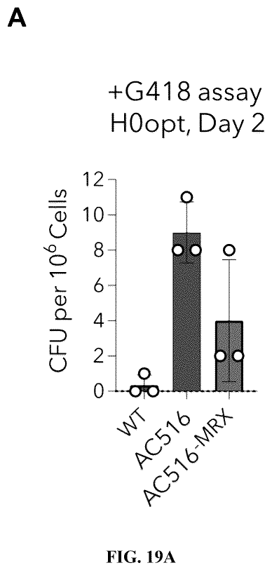

It was next determined whether the ability to generate combinatorial diversity could be applied to antibody fragments, as (i) a key function of V(D)J recombination is making antibodies, and (ii) yeast are an important platform for antibody sequence engineering and interrogation because of their ability display antibody fragments on their cell surface. The S. cerevisiae surface display strain EBY100 was engineered to co-express NLS-RAG1core , full-length RAG2, and HMGB1, as in BY4742. A new substrate plasmid was also expressed in this recombination/display yeast strain, which was designed similarly to the combinatorial GFP substrate plasmids but utilized two antibody fragments (scFvs) that were derived from the same heavy chain-light chain (VH-VL) pairing (FIG. 7A). The two scFvs differed in the amino acid sequence of their VH complementarity determining region 3 (CDR3), such that one scFv binds prostate stem cell antigen (PSCA), while the other binds glypican3 (GPC3). Both PSCA and GPC3 are antigenic targets known to be overexpressed in certain cancers. In the substrate plasmid, an AGA2 display protein fused to a DNA fragment of most of the scFv's VH is next to a 12-RSS, followed by an intersignal region and 23-RSSs preceding each 3′ fragment of the two scFvs. After inducing yeast cells harboring this scFv combinatorial diversity substrate, generation of yeast cell subpopulations was observed displaying functional anti-PSCA and anti-GPC3 binding scFvs (FIG. 7B and FIG. 18). In contrast, cells that did not have integrated RAG genes (BY4742) or cells cultured in glucose were unable to generate scFvs. As with GFP recombination, a higher rate of recombination was observed at the 23-RSS most proximal to the 12-RSS, in this case generating more αPSCA than αGPC3 scFvs. The present demonstration shows that antibody combinatorial diversity can be created by RAG proteins in vivo and functionally characterized in yeast.

Demonstrating True Coding Joint Formation in Yeast with Out Homology-Assisted Repair

The nucleotides in the coding DNA which are adjacent to the heptamer of each RSS can greatly impact the efficiency of recombination. These nucleotides can affect recombination efficiency at multiple points, including 1) DNA binding to the RAGI/RAG2/HMGB 1 complex and bending into the proper orientation, 2) RAG1 ssDNA cleavage and hairpin formation, and 3) hairpin opening and NHEJ repair. In particular, a G or C immediately adjacent to the heptamer has been shown to be much more favorable for DNA cleavage relative to an A or T at that position. It was contemplated to apply this to coding joint targets to improve the likelihood of forming coding joints that do not require homology for repair.

The initial target split-G418R plasmid that lacked coding end homology had T located 5′ of each RSS heptamer. Specifically, in the pY122-CJA-UP-HO plasmid, the coding DNA flanking the RSSs is as follows: 5′-TTGT′CACAGTG-3′ (SEQ ID NO: 37) at the 12-RSS and 5′-CACTGTG′AATT-3′ (SEQ ID NO: 38) at the 23-RSS. Note that for each sequence, the RAG1 cut site is marked with an apostrophe, and the RSS heptamer is underlined. No formation of coding joints were detected using this target plasmid (FIG. 3B). A new plasmid, pY112-CJA-U-H0opt that split the G418 resistance gene at a different location that positioned C and G 5′ of the heptamers was developed. The new sequences were as follows: 5′-GCGC′CACAGTG-3′ (SEQ ID NO: 39) at the 12-RSS and 5′-CACTGTG′CGGT-3′ (SEQ ID NO: 40) at the 23-RSS.

As a separate strategy to enable coding joint formation without homology, the present disclosure sought to improve repair post DNA cleavage. In yeast, the MRX complex, comprised of MRE11, RAD50, and XRS2, is essential to NHEJ repair and is responsible for end processing and tethering. These proteins have hairpin-opening activity under some circumstances and have been shown to be essential for signal joint formation in yeast. Therefore, these proteins were overexpressed to see if this would improve recombination by enhancing the repair of the double-strand breaks. A second copy of each protein driven by a strong constitutive promoter was integrated in strain AC516 (RAG1 (348-1006), RAG2, and HMGB1), which was our most active strain for homology-assisted coding joints (FIG. 3F), generating AC516-MRX.

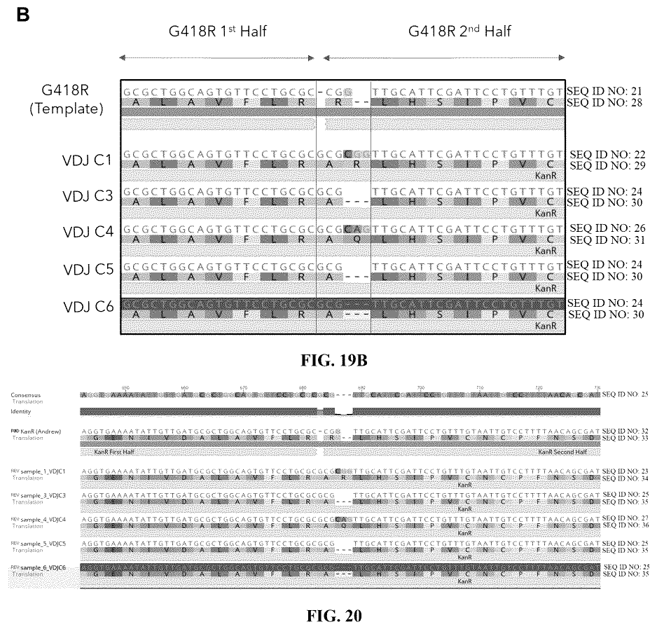

A G418 resistance recombination assay was performed with the new optimized target plasmid and compared AC516 with AC516-MRX. G418 resistant colonies were detected for the first time without relying on homology to repair the G418R marker. AC516 generated colonies at 9 CFUs per million cells (FIG. 19A). While this rate is nearly 1000-fold lower than plasmids which use 20-bp of homology, it demonstrated that the optimized plasmid was enabling true coding joint formation without requirement of any homology, as occurs in vertebrate B cells. The AC516-MRX strain had markedly lower rates of recombination than AC516. Since the coding ends have no homology, it remains that the DNA repair relies on MRX. Therefore, it is possible that the MRX complex is simply not a limiting factor in repair, and thus increasing its prevalence raises metabolic burden on the cell without improving repair.

To confirm that the colonies discovered were genuine coding joints, plasmid DNA was extracted from the cells and sequenced. A clear V(D)J coding joint had been formed in all six tested colonies, with each sequence also containing mutations introduced during the DNA repair process (FIG. 19B and FIG. 20). Up to three nucleotides were inserted, and three separate outcomes were detected over six colonies. There are additional repair outcomes that either result in a frame shift or incompatible codon which were not detected by the assay, showing that the real rate of true V(D)J recombination is higher than observed. These results are the first genuine coding joints made via V(D)J recombination in a non-vertebrate cell.

Because the DNA repair mechanism introduced mutations, it naturally generates junctional diversity that can be leveraged to generate libraries with highly variable regions between two conserved domains, such as the variable CDR3 between framework regions of heavy and light antibody domains. Therefore, the yeast cells can be used to continuously generate customized DNA libraries with both junctional and combinatorial diversity for any class of protein.

### Discussion

The present disclosure provides the engineering of a yeast platform able to perform homology-assisted V(D)J recombination, utilized protein-based strategies to enhance recombination rates, and explored how non-protein (e.g., substrate plasmid) factors influence recombination. Recombination is dependent on the presence and localization of RAGI and RAG2 and requires a functional 12-RSS and 23-RSS. By applying codon optimization, adding HMGB1, and using a truncated RAGI and full-length RAG2, the rate of homology-assisted coding joint recombination was increased over 500-fold from starting levels. Furthermore, using a separate, novel assay, cells that had generated signal joints in genomic DNA were successfully isolated. Signal joints are not dependent on homology. Therefore, their isolation demonstrates the capability for yeast to repair DNA using the NHEJ pathway, albeit at lower levels than the homology-assisted coding joints. It was then shown that recombination platform could quantitatively predict deleterious mutations in RAGI, a common cause of various immunodeficiency disorders. Finally, the platform was used to create the first synthetic instances of in vivo combinatorial diversity generation in two model protein classes: fluorescent proteins and antibodies.

In analyzing the impact of substrate sequences on recombination rates, a complex interplay between the transcriptional activity and length of the intersignal region was observed. While reducing the length between RSSs sometimes improved recombination (“U” and “P”), our shortest construct (“UT”) did not follow this trend. In addition, it was determined that, relative to the starting construct, removing the strong promoter in the intersignal region was beneficial for recombination in yeast. This promoter was originally included due to extensive work in mammalian cells showing the importance of RSS transcription in enabling V(D)J recombination. Another possible explanation for the impact of the intersignal region is that recombination is highly sensitive to nucleotides near the nonamer of the RSS, but this has not been reported previously. Moreover, in the “UT” and “U” target plasmids, the two nucleotides nearest each RSS are identical, showing transcription and length as more influential variables. Importantly, by engineering multiple recombination targets, selections were developed that allowed for isolation of yeast cells harboring coding joints or signal joints, which had not been done previously in microbial cells. In addition to these standard products of V(D)J recombination, a wide variety of aberrant DNA repair can sometimes occur, such as transposition events.

Yeast are well-established eukaryotic model cellular system that has been used to study several human disease states, including Huntington's disease, Alzheimer's disease, and cancer, through protein incorporation. Indeed, a variety of strategies exist to humanize yeast in ways that help study human disease. Herein, it shows that yeast cells expressing RAGI variants with pathogenic mutations demonstrated a decreased level of recombination rate that correlated with disease severity, expanding the use of yeast models towards immunodeficiencies. The RAG1 mutations tested should have a self-contained function, in that they are implicated directly in catalytic activity or DNA binding, and between the yeast system presented herein and prior the results showed gauging RAGI activity.

The present disclosure is the first demonstration of a cell being engineered to generate in vivo combinatorial protein diversity, which could be a useful tool to generate chimeric protein libraries. The RAG proteins have advantages over other site-specific nucleases which could conceivably be used to cleave DNA in vivo, such as CRISPR-Cas9 or meganucleases. With the RAG recombinase, both a 12-RSS and 23-RSS are required, and DSBs are simultaneously created exactly adjacent to two compatible gene segments. This increases the likelihood of productive recombination without increasing cell toxicity and further removes the RSS recognition sites from recombined DNA. Because further recombination is prevented when all RSSs of a given type are excised, the potential for continuous DNA cleavage and repair until the ‘smallest recombination option’ remains is eliminated.

The ability to generate chimeric proteins in vivo unlocks many possible design strategies. For instance, by including more fragments with 23-RSSs (and better tuning the RSS function), a small library of scFvs could be generated, and additional diversity could then be induced by another system designed for targeted mutagenesis. Because the recombination substrates are fully customizable, libraries could be generated that pair a light chain with various heavy chains, or vice versa, or fuse antibody gene segments together, such as, V gene segments with various DJ genes. Other classes of binders, such as designed ankyrin repeat proteins (DARPins) or nanobodies, could also be utilized in the present system.

An additional strategy contemplated herein to increase coding joint junctional diversity would be introduction of additional mammalian proteins, namely DNA-PKcs, Ku70, Ku80, and Artemis, that interact with the RAG complex and open hairpins in developing B cells. More simply, truncations of Artemis that are constitutively active in the absence of DNA-PKcs could open hairpins (and thereby assist coding joint formation) when expressed on their own. While the act of hairpin-opening results in a certain level of junctional diversity in B cells, one can introduce the TdT non-templated DNA polymerase into the present platform. Ultimately, engineering a yeast cell that undergoes V(D)J recombination as faithfully as possible is an excellent tool to study mammalian development and disease and is a powerful platform for synthetic biology and protein engineering.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLES

TABLE 1
Engineered Strains.

| Strain | Base | Modifications to Base Strain |
|---|---|---|
| BY4742- | BY4742 | YPRCΔ15::pNAB2-NAB2-mCherry-tADH1, psmTEF1- |
| NAB2 | | snNAT-tCYC1 |
| BY4742- | BY4742 | YPRCΔ15::pNOP56-NOP56-mCherry-tADH1, psmTEF1- |
| NOP56 | | snNAT-tCYC1 |
| AC411 | BY4742- | YORWΔ22::pGAL1-RAG1-eGFP-tPRM9, pagTEF1-klLEU2- |
| | NAB2 | tagTEF1 |
| AC412 | BY4742- | YORWΔ22::pGAL1-RAG1core-eGFP-tPRM9, pagTEF1- |
| | NAB2 | klLEU2- |
| | | tagTEF1 |
| AC413 | BY4742- | YORWΔ22::pGAL1-hht1NLS-RAG1core-eGFP-tPRM9, |
| | NAB2 | pagTEF1- |
| | | klLEU2-tagTEF1 |
| AC414 | BY4742- | YORWΔ22::pGAL1-RAG2-eGFP-tPRM9, pagTEF1-klLEU2- |
| | NAB2 | tagTEF1 |
| AC415 | BY4742- | YORWΔ22::pGAL1-RAG2core-eGFP-tPRM9, pagTEF1- |
| | NAB2 | klLEU2- |
| | | tagTEF1 |
| AC416 | BY4742- | YORWΔ22::pGAL1-hht1NLS-RAG2core-eGFP-tPRM9, |
| | NAB2 | pagTEF1- |
| | | klLEU2-tagTEF1 |
| AC421 | BY4742- | YORWΔ22::pGAL1-RAG1-eGFP-tPRM9, pagTEF1-klLEU2- |
| | NOP56 | tagTEF1 |
| AC422 | BY4742- | YORWΔ22::pGAL1-RAG1core-eGFP-tPRM9, pagTEF1- |
| | NOP56 | klLEU2- |
| | | tagTEF1 |
| AC423 | BY4742- | YORWΔ22::pGAL1-hht1NLS-RAG1core-eGFP-tPRM9, |
| | NOP56 | pagTEF1- |
| | | klLEU2-tagTEF1 |
| AC424 | BY4742- | YORWΔ22::pGAL1-RAG2-eGFP-tPRM9, pagTEF1-klLEU2- |
| | NOP56 | tagTEF1 |
| AC425 | BY4742- | YORWΔ22::pGAL1-RAG2core-eGFP-tPRM9, pagTEF1- |
| | NOP56 | klLEU2- |
| | | tagTEF1 |
| AC426 | BY4742- | YORWΔ22::pGAL1-hht1NLS-RAG2core-eGFP-tPRM9, |
| | NOP56 | pagTEF1- |
| | | klLEU2-tagTEF1 |
| AC427 | BY4742- | YORWΔ22::pGAL1-RAG1(1-1008)-eGFP-tPRM9, pagTEF1- |
| | NAB2 | klLEU2-tagTEF1 |
| AC428 | BY4742- | YORWΔ22::pGAL1-RAG1(215-1008)-eGFP-tPRM9, |
| | NAB2 | pagTEF1- |
| | | klLEU2-tagTEF1 |
| AC429 | BY4742- | YORWΔ22::pGAL1-RAG1(284-1006)-eGFP-tPRM9, |
| | NAB2 | pagTEF1- |
| | | klLEU2-tagTEF1 |
| AC430 | BY4742- | YORWΔ22::pGAL1-RAG1(348-1006)-eGFP-tPRM9, |
| | NAB2 | pagTEF1- |
| | | klLEU2-tagTEF1 |
| AC431 | BY4742- | YORWΔ22::pGAL1-hht1NLS-RAG1core-eGFP-tPRM9, |
| | NAB2 | pagTEF1- |
| | | klLEU2-tagTEF1 |
| AC432 | BY4742- | YORWΔ22::pGAL1-hsRAG1(387-1011)-eGFP-tPRM9, |
| | NAB2 | pagTEF1- |
| | | klLEU2-tagTEF1 |
| AC433 | BY4742- | YORWΔ22::pGAL2-hsRAG2-eGFP-tPRM9, pagTEF1- |
| | NAB2 | klLEU2- |
| | | tagTEF1 |
| AC434 | BY4742- | YORWΔ22::pGAL2-RAG2-eGFP-tPRM9, pagTEF1-klLEU2- |
| | NAB2 | tagTEF1 |
| AC501 | BY4742 | YPRCτ3::pGAL1-RAG1-tPRM9, pGAL2-RAG2-tRPL15A, |
| | | psmTEF1-snNAT-tagTEF1 |
| AC502 | BY4742 | YPRCτ3::pGAL1-RAG1-tPRM9, pGAL2-RAG2-tRPL15A, |
| | | psmTEF1-snNAT-tagTEF1, pspTDH3-HMGB1-tRPL41B |
| AC503 | BY4742 | YPRCτ3::pGAL1-RAG1core-tPRM9, pGAL2-RAG2core- |
| | | tRPL15A, |
| | | psmTEF1-snNAT-tagTEF1 |
| AC504 | BY4742 | YPRCτ3::pGAL1-RAG1core-tPRM9, pGAL2-RAG2core- |
| | | tRPL15A, psmTEF1-snNAT-tagTEF1, pspTDH3-HMGB1- |
| | | tRPL41B |
| AC505 | BY4742 | YPRCτ3::pGAL1-RAG1core-tPRM9, pGAL2-RAG2- |
| | | tRPL15A, |
| | | psmTEF1-snNAT-tagTEF1 |
| AC506 | BY4742 | YPRCτ3::pGAL1-RAG1core-tPRM9, pGAL2-RAG2- |
| | | tRPL15A, psmTEF1-snNAT-tagTEF1, pspTDH3-HMGB1- |
| | | tRPL41B |
| AC507 | BY4742 | YPRCτ3::pGAL1-RAG1core-tPRM9, pGAL2-hht1NLS- |
| | | RAG2core- |
| | | tRPL15A, psmTEF1-snNAT-tagTEF1 |
| AC508 | BY4742 | YPRCτ3::pGAL1-hht1NLS-RAG1core-tPRM9, pGAL2- |
| | | hht1NLS- |
| | | RAG2core-tRPL15A, psmTEF1-snNAT-tagTEF1 |
| AC509 | BY4742 | YPRCτ3::pGAL1-hht1NLS-RAG1core-tPRM9, pGAL2- |
| | | hht1NLS- |
| | | RAG2core-tRPL15A, psmTEF1-snNAT-tagTEF1, pspTDH3- |
| | | HMGB1-tRPL41B |
| AC510 | BY4742 | YPRCτ3::pGAL1-wtRAG1core-tPRM9, pGAL2-wtRAG2core- |
| | | tRPL15A, psmTEF1-snNAT-tagTEF1 |
| AC511 | BY4742 | YPRCτ3::pGAL1-wtRAG1core-tPRM9, pGAL2-wtRAG2core- |
| | | tRPL15A, psmTEF1-snNAT-tagTEF1, pspTDH3-HMGB1- |
| | | tRPL41B |
| AC512 | BY4742 | YPRCτ3::pGAL1-hsRAG1(387-1011)-tPRM9, pGAL2- |
| | | hsRAG2-tRPL15A, psmTEF1-snNAT-tagTEF1, pspTDH3- |
| | | HMGB1-tRPL41B |
| AC513 | BY4742 | YPRCτ3::pGAL1-RAG1(1-1008)-tPRM9, pGAL2-RAG2- |
| | | tRPL15A, psmTEF1-snNAT-tagTEF1, pspTDH3-HMGB1- |
| | | tRPL41B |
| AC514 | BY4742 | YPRCτ3::pGAL1-RAG1(215-1008)-tPRM9, pGAL2-RAG2- |
| | | tRPL15A, psmTEF1-snNAT-tagTEF1, pspTDH3-HMGB1- |
| | | tRPL41B |
| AC515 | BY4742 | YPRCτ3::pGAL1-RAG1(284-1006)-tPRM9, pGAL2-RAG2- |
| | | tRPL15A, psmTEF1-snNAT-tagTEF1, pspTDH3-HMGB1- |
| | | tRPL41B |
| AC516 | BY4742 | YPRCτ3::pGAL1-RAG1(348-1006)-tPRM9, pGAL2-RAG2- |
| | | tRPL15A, psmTEF1-snNAT-tagTEF1, pspTDH3-HMGB1- |
| | | tRPL41B |
| AC517 | BY4742 | YPRCτ3::pGAL1-RAG1core-tPRM9, pGAL2-RAG2(T490A)- |
| | | tRPL15A, psmTEF1-snNAT-tagTEF1, pspTDH3-HMGB1- |
| | | tRPL41B |
| AC518 | BY4742 | YPRCτ3::pGAL1-hht1NLS-RAG1core-tPRM9, pGAL2-RAG2- |
| | | tRPL15A, psmTEF1-snNAT-tagTEF1, pspTDH3-HMGB1- |
| | | tRPL41B |
| AC519 | BY4742 | YPRCτ3::pGAL1-RAG1(348-1008)-tPRM9, pGAL2-RAG2- |
| | | tRPL15A, psmTEF1-snNAT-tagTEF1, pspTDH3-HMGB1- |
| | | tRPL41B |
| AC520 | BY4742 | YPRCτ3::pGAL1-RAG1(348-1008)D708N -tPRM9, pGAL2- |
| | | RAG2-tRPL15A, psmTEF1-snNAT-tagTEF1, pspTDH3- |
| | | HMGB1-tRPL41B |
| AC521 | BY4742 | YPRCτ3::pGAL1-RAG1(348-1008)E962Q-tPRM9, pGAL2- |
| | | RAG2-tRPL15A, psmTEF1-snNAT-tagTEF1, pspTDH3- |
| | | HMGB1-tRPL41B |
| AC522 | BY4742 | YPRCτ3::pGAL1-RAG1(348-1008)R855A/R856A -tPRM9, |
| | | pGAL2-RAG2-tRPL15A, psmTEF1-snNAT-tagTEF1, |
| | | pspTDH3-HMGB1-tRPL41B |
| AC523 | BY4742 | YPRCτ3::pGAL1-RAG1(348-1008)R894A-tPRM9, pGAL2- |
| | | RAG2-tRPL15A, psmTEF1-snNAT-tagTEF1, pspTDH3- |
| | | HMGB1-tRPL41B |
| AC518-SJ | AC518 | NRT1/GYP1::SJUEout |
| BY4742-SJ | BY4742 | NRT1/GYP1::SJUEout |
| AC518-CJA | AC518 | NRT1/GYP1::CJA-UP-H20 |
| BY4742-CJA | BY4742 | NRT1/GYP1::CJA-UP-H20 |
| AC518- | AC518 | YORWΔ22::pTEF1-hph-tagTEF1, pFBA1-hsArtemis(1-413)- |
| Art413 | | tVMA2 |
| BY4742- | BY4742 | YORWΔ22::pTEF1-hph-tagTEF1, pFBA1-hsArtemis(1-413)- |
| Art413 | | tVMA2 |
| AC601 | BY4742 | YPRCτ3::pGAL1-RAG1core(T400P)-tPRM9, pGAL2-RAG2- |
| | | tRPL15A, psmTEF1-snNAT-tagTEF1, pspTDH3-HMGB1- |
| | | tRPL41B |
| AC602 | BY4742 | YPRCτ3::pGAL1-RAG1core(G513A)-tPRM9, pGAL2-RAG2- |
| | | tRPL15A, psmTEF1-snNAT-tagTEF1, pspTDH3-HMGB1- |
| | | tRPL41B |
| AC603 | BY4742 | YPRCτ3::pGAL1-RAG1core(R556S)-tPRM9, pGAL2-RAG2- |
| | | tRPL15A, psmTEF1-snNAT-tagTEF1, pspTDH3-HMGB1- |
| | | tRPL41B |
| AC604 | BY4742 | YPRCτ3::pGAL1-RAG1core(R696Q)-tPRM9, pGAL2-RAG2- |
| | | tRPL15A, psmTEF1-snNAT-tagTEF1, pspTDH3-HMGB1- |
| | | tRPL41B |
| AC605 | BY4742 | YPRCτ3::pGAL1-RAG1core(K989E)-tPRM9, pGAL2-RAG2- |
| | | tRPL15A, psmTEF1-snNAT-tagTEF1, pspTDH3-HMGB1- |
| | | tRPL41B |
| AC701 | EBY100 | YPRCτ3::pGAL1-hht1NLS-RAG1core-tPRM9, pGAL2-RAG2- |
| | | tRPL15A, psmTEF1-snNAT-tagTEF1, pspTDH3-HMGB1- |
| | | tRPL41B |

Note:
All promoters and terminators are from S. cerevisiae unless otherwise noted. All RAG genes are yeast-codon-optimized mouse sequences except where noted. RAG1core includes residues 383-1006 and RAG2core includes residues 1-383. Abbreviations: ag = Ashbya gossypii, hs = Homo sapiens, kl = Kluyveromyces lactis, nc = Naumovozyma castellii, NLS = nuclear localization signal, sm = Saccharomyces mikatae, sn = Streptomyces noursei, wt = wild-type sequence

TABLE 2
Recombination target and associated plasmids constructed.

| Name | Brief Description |
|---|---|
| pY112-CJA-UP-H20 | Split antibiotic resistance recombination, 20-bp homology |
| pY112-CJA-UP- | Split antibiotic resistance recombination, 20-bp homology, |
| H20-M12 | mutated 12 RSS |
| pY112-CJA-UP- | Split antibiotic resistance recombination, 20-bp homology, |
| H20-M23 | mutated 23-RSS |
| pY112-CJA-UP- | Split antibiotic resistance recombination, 20-bp homology, |
| H20-MBoth | mutated 12- and 23-RSS |
| pY112-CJA-UP- | Split antibiotic resistance recombination, 20-bp homology, two |
| H20-Double12 | 12-RSSs |
| pY112-CJA-UP- | Split antibiotic resistance recombination, 20-bp homology, two |
| H20-Double23 | 23-RSSs |
| pY112-CJA-UP-H0 | Split antibiotic resistance recombination, 0-bp homology |
| pY112-CJA-UP-H3 | Split antibiotic resistance recombination, 3-bp homology |
| pY112-CJA-UP-H6 | Split antibiotic resistance recombination, 6-bp homology |
| pY112-CJA-UP-H10 | Split antibiotic resistance recombination, 10-bp homology |
| pY112-CJA-UP-H15 | Split antibiotic resistance recombination, 15-bp homology |
| pY112-CJA-UP-H40 | Split antibiotic resistance recombination, 40-bp homology |
| pY122-CJA-UP-H20 | Split antibiotic resistance recombination, 20-bp homology, 2μ |
| | ORI (high copy) |
| pY112-CJA-UPalt- | Split antibiotic resistance recombination, 20-bp homology, |
| H20 | different split site |
| pY112-CJA-U-H20 | Split antibiotic resistance recombination, 20-bp homology, |
| | promoter removed relative to pY112-CJA-UP-H20 |
| pY112-CJA-P-H20 | Split antibiotic resistance recombination, 20-bp homology, Ura3 |
| | cassette removed relative to pY112-CJA-UP-H20 |
| pY112-CJA-UF-H20 | Split antibiotic resistance recombination, 20-bp homology, |
| | spacer in pY112-CJA-UP-H20 replaced with URA3 fragment |
| pY112-CJCG-ES- | Split GFP recombination, 20-bp homology, second half |
| H20 | fragment order: eGFP then Sapphire |
| pY112-CJCG-SE- | Split GFP recombination, 20-bp homology, second half |
| H20 | fragment order: Sapphire then eGFP |
| pY112-CJCG-EA- | Split GFP recombination, 20-bp homology, second half |
| H20 | fragment order: eGFP then Azurite |
| pY112-CJCG-ESA- | Split GFP recombination, 20-bp homology, second half |
| H20 | fragment order: eGFP then Sapphire then Azurite |
| pY112-psmTEF1- | Single-color control for GFP recombination, eGFP |
| eGFP | |
| pY112-psmTEF1- | Single-color control for GFP recombination, Sapphire |
| Sapphire | |
| pY112-psmTEF1- | Single-color control for GFP recombination, Azurite |
| Azurite | |
| pY112-SJUE | Signal joint target assay, URA3 and eGFP cassettes flanked by |
| | 12-and 23-RSS |
| pY112-CJA-U-H0opt | SEQ ID NO: 48 |

TABLE 3
RSS Sequences.

| | Sequence | SEQ |
|---|---|---|
| | (5′ heptamer to 3′ | ID |
| Name | nonamer) | NO. |
| 12-RSS | CACAGTGCTACAGACTGGAACA | 6 |
| | AAAACC | |
| 23-RSS | CACAGTGGTAGTACTCCACTGTC | 7 |
| | TGGCTGTACAAAAACC | |
| 12-RSS-mut | GTACAGCCTACAGACTGGACAG | 8 |
| | TCAGTA | |
| 23-RSS-mut | GTACAGCGTAGTACTCCACTGTC | 9 |
| | TGGCTGTCAGTCAGTA | |
| 23-RSS-2 (used in split eGFP | CACAGTGTGAAAACCCACATCCT | 10 |
| plasmids) | GAGAGTGACAAAAACC | |
| 23-RSS-3 (used in split eGFP/scFv | CACAGTGAGGGGAAGTCATTGT | 11 |
| plasmids) | GAGCCCAGACAAAAACC | |
| 12-RSS-heptamer-region | TTGT'CACAGTG | 37 |
| (Apostrophe represents cut site; | | |
| underlined nucleotides represent RSS | | |
| heptamer) | | |
| 23-RSS-heptamer-region | CACTGTG'AATT | 38 |
| (Apostrophe represents cut site; | | |
| underlined nucleotides represent RSS | | |
| heptamer) | | |
| 12-RSS-heptamer-region-modified | GCGC'CACAGTG | 39 |
| (Apostrophe represents cut site; | | |
| underlined nucleotides represent RSS | | |
| heptamer) | | |
| 23-RSS-heptamer-region-modified | CACTGTG'CGGT | 40 |
| (Apostrophe represents cut site; | | |
| underlined nucleotides represent RSS | | |
| heptamer) | | |
| 12-RSS H0opt mutant | GCGCCACAGTGCTACAGACTGGA | 41 |
| | ACAAAAACC | |
| 23-RSS H0opt mutant | ACCGCACAGTGGTAGTACTCCAC | 42 |
| | TGTCTGGCTGTACAAAAACC | |

SEQUENCES
1.
RAG1_mouse
SEQ ID NO: 1
ATGGCTGCCTCCTTGCCGTCTACCTTGAGCTTCAGTTCTGCACCCGATGAAATTCAAC
ATCCACAAATCAAATTTTCCGAGTGGAAATTTAAGCTGTTTAGGGTTAGATCCTTTGA
AAAGGCACCCGAAGAAGCACAAAAGGAAAAAGATTCCTCAGAAGGGAAACCTTACCTA
GAACAATCTCCAGTAGTTCCAGAGAAGCCTGGTGGCCAAAACTCAATTTTGACTCAAC
GTGCATTGAAATTGCATCCTAAATTTTCAAAGAAATTTCATGCTGATGGGAAGTCAAG
CGACAAAGCAGTTCACCAAGCCAGGTTGAGACATTTCTGTAGAATCTGTGGTAATCGT
TTCAAGAGTGACGGTCACTCAAGAAGATACCCAGTTCATGGTCCCGTTGACGCTAAAA
CCCAAAGTCTTTTCCGTAAAAAGGAAAAAAGAGTAACTTCCTGGCCAGACTTGATTGC
CAGGATTTTCAGAATCGACGTAAAAGCAGATGTTGACTCCATTCACCCGACGGAATTC
TGCCATGATTGTTGGAGCATCATGCATAGAAAGTTCTCTAGTTCTCACAGTCAGGTTT
ACTTCCCAAGGAAAGTAACTGTGGAGTGGCACCCCCACACACCGTCCTGTGACATTTG
TTTTACTGCACATAGAGGATTGAAGAGGAAGAGACATCAGCCCAATGTACAGCTATCT
AAGAAACTAAAAACTGTGTTGAACCACGCGAGAAGGGATCGTAGAAAGAGAACTCAAG
CTAGGGTCAGTTCAAAGGAAGTCCTAAAGAAGATCTCCAACTGCAGTAAGATTCATTT
GAGTACAAAGCTTCTTGCCGTGGACTTCCCAGCACACTTTGTGAAATCCATCTCCTGC
CAAATATGCGAACACATTTTAGCTGATCCCGTTGAGACAAGCTGCAAGCATCTATTCT
GTCGTATTTGCATTCTGAGATGTTTAAAAGTTATGGGCTCTTATTGTCCCTCTTGCAG
GTATCCGTGCTTCCCTACTGACTTAGAAAGCCCAGTTAAGTCCTTTTTAAACATTTTG
AATTCTCTGATGGTTAAGTGTCCCGCGCAAGATTGCAATGAGGAAGTGAGTTTAGAAA
AATATAACCACCATGTTTCAAGTCACAAAGAATCTAAAGAGACTTTGGTTCATATCAA
TAAAGGTGGAAGACCTAGACAACATCTACTGTCATTAACGAGAAGGGCGCAGAAACAT
AGATTAAGGGAGTTAAAGATTCAAGTTAAAGAATTTGCTGACAAAGAAGAAGGTGGGG
ATGTAAAAGCTGTTTGCTTGACATTGTTTCTACTGGCACTGAGGGCGAGGAATGAACA
CAGGCAAGCTGATGAATTAGAGGCCATAATGCAAGGCAGGGGATCCGGTCTTCAACCA
GCTGTTTGCTTGGCTATCCGTGTCAATACTTTTCTTAGCTGTTCTCAATATCATAAGA
TGTATAGGACTGTTAAAGCTATAACTGGTAGGCAAATTTTTCAACCTTTGCATGCTCT
TAGAAATGCCGAAAAAGTTCTTTTACCAGGTTACCATCCATTTGAGTGGCAACCTCCA
TTGAAGAATGTTTCCTCTAGAACTGATGTTGGAATAATTGATGGTCTGTCTGGATTGG
CATCTTCTGTTGATGAATATCCAGTAGATACGATTGCGAAAAGGTTCAGATACGATTC
TGCTTTGGTTTCTGCTTTGATGGATATGGAAGAAGACATCTTGGAAGGCATGAGATCC
CAAGATCTTGATGACTACTTGAATGGTCCATTTACAGTTGTTGTAAAGGAATCTTGCG
ATGGAATGGGAGATGTGAGTGAAAAGCACGGAAGTGGGCCTGCAGTTCCAGAAAAGGC
GGTTCGTTTCTCTTTCACAGTTATGAGAATTACGATAGAACATGGTTCACAAAACGTG
AAGGTTTTTGAGGAACCCAAGCCTAATTCTGAATTATGTTGCAAGCCGTTGTGTCTTA
TGTTGGCAGATGAGTCTGACCATGAGACACTTACTGCTATTCTAAGCCCCCTAATTGC
TGAGAGGGAAGCCATGAAGAGTAGTGAATTAACGCTAGAGATGGGAGGCATCCCGAGG
ACTTTTAAATTCATATTCAGGGGTACTGGATACGATGAAAAACTTGTCAGGGAAGTAG
AAGGTTTGGAAGCTTCTGGCTCAGTATATATCTGTACATTGTGTGATACCACCCGTTT
GGAAGCCTCTCAAAATCTTGTCTTCCACTCCATAACGAGAAGCCACGCAGAGAACCTG
CAGAGATATGAGGTCTGGAGATCAAATCCGTATCATGAGTCCGTGGAAGAGTTGCGTG
ACAGAGTGAAAGGTGTCTCTGCCAAACCTTTCATCGAAACAGTTCCTTCAATAGATGC
GCTTCACTGTGATATTGGTAATGCAGCTGAATTTTATAAGATTTTCCAGCTGGAGATA
GGTGAAGTATATAAACATCCTAATGCCTCTAAAGAAGAAAGAAAGAGATGGCAAGCCA
CGTTAGATAAACATCTTAGGAAAAGAATGAACTTAAAACCAATCATGAGGATGAATGG
TAACTTTGCCAGAAAGTTGATGACTCAAGAGACTGTAGATGCAGTTTGTGAATTAATT
CCTTCTGAGGAGAGGCATGAAGCTTTGAGGGAGTTGATGGACTTATATCTAAAAATGA
AACCAGTGTGGAGATCTTCATGTCCAGCTAAAGAATGTCCAGAGAGTTTGTGTCAATA
CAGTTTCAACTCACAGCGTTTCGCGGAACTACTGAGTACCAAGTTTAAATATAGATAT
GAAGGTAAAATAACTAATTACTTTCATAAAACCTTGGCACATGTCCCTGAAATTATTG
AAAGGGATGGTTCTATCGGGGCATGGGCAAGTGAAGGAAATGAAAGCGGTAACAAATT
GTTTAGAAGATTTAGGAAAATGAATGCCAGGCAGTCTAAGTGCTATGAGATGGAAGAT
GTCCTGAAACATCACTGGTTGTATACTTCAAAATACTTACAGAAGTTTATGAATGCTC
ATAACGCGTTAAAAAGCTCTGGATTTACCATGAACTCAAAGGAGACATTAGGGGACCC
TTTGGGAATTGAGGATTCTCTGGAAAGCCAAGATTCAATGGAGTTTTAA
2.
RAG2_mouse
SEQ ID NO: 2
ATGTCCCTGCAGATGGTAACAGTGGGTCATAACATAGCGTTAATTCAACCAGGTTTCT
CACTTATGAATTTTGATGGCCAAGTTTTCTTCTTTGGCCAAAAAGGCTGGCCAAAGCG
TTCCTGTCCTACTGGTGTCTTTCATTTTGATATAAAACAAAATCATTTAAAACTGAAG
CCTGCAATCTTCTCTAAAGATTCCTGCTACCTACCACCTTTGCGTTACCCAGCTACTT
GCTCATACAAAGGTAGCATAGACTCTGACAAGCATCAATATATCATTCACGGTGGGAA
GACACCGAACAATGAGCTATCCGATAAGATTTATATCATGTCTGTTGCTTGCAAGAAT
AACAAAAAAGTTACTTTCCGTTGCACAGAGAAAGACTTAGTAGGTGATGTCCCTGAAC
CAAGATACGGCCATAGCATTGACGTGGTGTATAGTAGAGGTAAAAGCATGGGTGTTCT
GTTTGGAGGTAGGTCATACATGCCTTCTACCCAACGTACCACAGAAAAATGGAATAGT
GTAGCTGACTGTCTACCCCATGTTTTCTTGATAGATTTTGAATTTGGTTGTGCTACAT
CATATATTTTGCCAGAACTTCAAGATGGGCTGTCTTTTCATGTTTCTATTGCCAGAAA
CGATACGGTTTATATTTTGGGGGGACACTCATTAGCCAGTAATATAAGACCTGCTAAC
TTGTATCGTATAAGAGTGGACCTTCCACTGGGTACCCCAGCAGTGAATTGTACAGTCT
TGCCAGGAGGAATCTCTGTATCCAGTGCGATCTTAACTCAAACAAACAATGATGAATT
TGTTATTGTGGGTGGTTATCAGCTGGAAAATCAGAAAAGGATGGTCTGTAGCTTGGTT
TCTCTAGGTGACAACACGATTGAAATCAGTGAGATGGAGACTCCAGACTGGACGTCAG
ATATTAAGCATAGCAAAATATGGTTTGGAAGCAACATGGGAAACGGTACTATTTTCCT
TGGCATACCAGGAGACAATAAGCAGGCTATGTCAGAAGCATTCTATTTCTATACTTTG
AGATGCTCTGAAGAGGATTTGAGTGAAGATCAGAAAATTGTCTCCAACAGTCAAACAT
CAACAGAAGATCCTGGTGACTCCACTCCCTTTGAAGACTCAGAGGAATTTTGTTTCAG
TGCTGAAGCAACCAGTTTTGATGGTGACGATGAATTTGACACCTACAATGAAGATGAT
GAAGATGACGAGTCTGTAACCGGCTACTGGATAACATGTTGTCCGACTTGTGATGTTG
ACATCAATACCTGGGTTCCGTTCTATTCAACGGAGTTAAATAAACCAGCCATGATCTA
TTGTTCTCATGGTGATGGGCACTGGGTACATGCCCAATGCATGGATTTGGAAGAAAGA
ACATTGATCCACTTGTCAGAAGGAAGCAACAAGTATTATTGCAATGAACATGTACAAA
TAGCAAGAGCATTGCAAACTCCCAAAAGAAACCCCCCCTTACAAAAACCTCCAATGAA
ATCCTTGCACAAAAAAGGCTCTGGTAAAGTCTTGACTCCTGCCAAGAAATCCTTCCTT
AGAAGACTGTTTGATTAA
3.
RAG1_human
SEQ ID NO: 3
ATGGCTGCTTCATTTCCACCCACATTGGGTTTATCAAGTGCTCCCGACGAAATTCAAC
ACCCACATATAAAATTTTCTGAGTGGAAATTCAAGTTATTCAGGGTGAGATCCTTTGA
GAAAACACCTGAGGAAGCACAAAAGGAAAAAAAGGACAGTTTCGAAGGTAAGCCATCT
TTAGAACAGTCACCCGCAGTCTTAGATAAAGCAGATGGCCAAAAACCAGTTCCCACTC
AACCATTGCTAAAGGCTCATCCAAAATTTAGTAAGAAATTCCACGACAACGAAAAAGC
TAGGGGCAAAGCCATACACCAGGCCAATTTAAGGCATTTGTGCAGAATCTGTGGTAAC
TCTTTTAGAGCAGACGAGCATAACCGTAGATATCCCGTCCACGGCCCCGTAGACGGAA
AAACTTTAGGACTATTAAGGAAGAAGGAAAAACGTGCCACCTCTTGGCCTGATTTAAT
CGCTAAAGTGTTCAGGATTGACGTCAAAGCTGACGTAGATTCCATCCATCCCACTGAG
TTCTGTCATAATTGTTGGTCAATAATGCATCGTAAGTTCTCCTCTGCCCCCTGTGAAG
TATATTTCCCAAGGAACGTAACAATGGAATGGCACCCACATACACCCTCCTGCGACAT
ATGTAACACCGCAAGAAGAGGATTGAAAAGAAAATCATTGCAACCCAATTTACAACTA
AGTAAGAAGTTAAAGACCGTATTGGATCAAGCCAGGCAGGCTAGACAAAGAAAGAGAC
GTGCACAAGCAAGGATCTCCTCTAAAGACGTGATGAAGAAAATAGCTAACTGTTCAAA
AATTCACTTATCAACCAAATTATTAGCCGTCGATTTCCCAGAGCATTTCGTCAAATCA
ATATCTTGTCAAATTTGTGAACACATTTTAGCTGATCCAGTAGAAACCAATTGTAAAC
ATGTCTTTTGTAGGGTTTGCATTTTGAGATGCTTAAAGGTTATGGGTTCTTACTGTCC
ATCATGCAGATACCCATGCTTCCCTACTGATTTGGAAAGTCCTGTGAAGAGTTTTTTG
TCTGTCTTGAATTCCCTAATGGTCAAATGCCCTGCAAAGGAGTGTAATGAAGAGGTAT
CATTAGAAAAATATAACCATCACATATCCTCACATAAAGAAAGTAAAGAGATCTTCGT
GCACATTAATAAGGGCGGACGTCCAAGGCAGCATTTGTTGTCTTTAACTAGGAGAGCA
CAAAAACACAGGTTGAGAGAATTAAAATTGCAAGTCAAAGCTTTCGCTGACAAGGAAG
AGGGCGGTGATGTTAAGTCAGTTTGTATGACATTATTCTTGTTAGCATTGAGAGCCAG
AAATGAACACAGGCAGGCTGATGAGTTGGAAGCCATAATGCAAGGAAAAGGTTCTGGC
CTACAGCCTGCTGTCTGCCTAGCCATCAGAGTAAATACTTTCTTGTCATGTTCCCAAT
ATCACAAGATGTATAGAACTGTTAAAGCTATCACAGGTAGGCAAATTTTTCAACCATT
GCATGCATTGAGGAACGCAGAAAAGGTGTTGTTACCAGGTTACCACCACTTTGAATGG
CAACCTCCTTTAAAGAACGTATCTTCCTCTACCGACGTGGGTATAATCGACGGATTGA
GTGGCTTGTCATCAAGTGTGGACGACTATCCTGTAGATACTATTGCCAAAAGGTTTAG
ATATGATTCCGCCTTGGTATCTGCATTGATGGACATGGAAGAGGATATCCTAGAGGGT
ATGAGGTCACAGGACTTGGATGATTATTTGAATGGTCCTTTCACAGTAGTAGTTAAAG
AAAGTTGCGACGGCATGGGCGACGTATCAGAAAAACATGGTTCCGGACCTGTTGTCCC
AGAAAAGGCTGTGAGGTTCTCTTTTACAATCATGAAGATAACAATAGCACATTCTAGT
CAAAATGTAAAGGTGTTTGAAGAAGCTAAGCCAAACTCCGAATTATGTTGCAAGCCTT
TATGCCTAATGTTAGCCGATGAAAGTGATCACGAAACATTAACCGCTATCTTATCTCC
ACTAATTGCTGAAAGGGAAGCTATGAAGTCATCAGAGTTAATGTTGGAGTTGGGTGGT
ATCTTGAGAACTTTCAAATTCATCTTTAGAGGAACAGGTTACGACGAAAAGTTGGTTA
GAGAAGTGGAAGGTTTAGAAGCTTCCGGATCAGTATATATTTGTACTTTGTGCGATGC
AACTAGATTAGAAGCTAGTCAAAACTTAGTCTTTCATTCAATAACCAGATCACATGCT
GAGAACTTAGAGAGGTATGAAGTATGGAGGTCCAACCCCTATCATGAATCTGTTGAAG
AGTTGAGAGATAGAGTCAAGGGTGTTAGTGCAAAACCTTTCATAGAAACCGTTCCATC
CATTGACGCTTTACACTGTGACATAGGTAATGCAGCCGAGTTTTACAAGATATTCCAG
TTAGAGATAGGTGAGGTATATAAAAATCCAAACGCCTCTAAAGAAGAAAGAAAGAGAT
GGCAGGCCACACTAGATAAACACCTAAGAAAGAAGATGAATTTAAAGCCCATCATGAG
GATGAACGGTAACTTTGCACGTAAACTAATGACAAAGGAAACTGTTGACGCCGTATGC
GAACTAATCCCTTCCGAAGAAAGGCATGAGGCATTGAGAGAATTGATGGACTTGTATT
TGAAAATGAAGCCTGTCTGGCGTAGTTCTTGCCCTGCTAAGGAATGTCCAGAATCACT
ATGTCAGTATTCATTCAATTCTCAGAGATTTGCAGAGTTGTTATCCACAAAGTTCAAA
TATCGTTATGAAGGAAAGATTACAAATTATTTTCACAAAACATTGGCACATGTCCCAG
AAATAATAGAGAGGGATGGTTCCATAGGAGCATGGGCCTCTGAAGGAAACGAATCAGG
AAACAAACTATTCCGTAGATTCAGAAAAATGAACGCAAGACAGAGTAAGTGCTATGAG
ATGGAAGATGTGCTAAAGCATCACTGGCTATATACATCTAAGTACCTACAGAAGTTTA
TGAACGCCCACAATGCATTGAAAACAAGTGGATTTACCATGAATCCTCAAGCCTCCTT
GGGAGATCCATTGGGTATTGAGGACTCCTTAGAATCTCAAGATTCCATGGAATTCTAA
4.
RAG2_human
SEQ ID NO: 4
ATGAGTCTACAGATGGTCACTGTCTCAAACAATATTGCATTAATTCAGCCTGGATTTT
CTTTAATGAATTTCGATGGTCAAGTTTTTTTCTTCGGCCAGAAAGGTTGGCCTAAGAG
ATCATGTCCCACCGGAGTCTTCCATTTAGACGTCAAGCATAATCACGTAAAGTTGAAG
CCCACAATTTTCTCCAAGGATAGTTGCTACCTACCTCCACTAAGATATCCTGCCACAT
GCACCTTCAAAGGATCACTAGAGTCTGAAAAACACCAATACATCATTCATGGTGGAAA
AACTCCAAATAATGAGGTTTCCGATAAAATTTATGTAATGTCCATTGTTTGCAAAAAC
AATAAGAAAGTTACTTTTAGATGTACCGAAAAAGATTTGGTTGGAGACGTGCCAGAGG
CAAGATACGGTCATAGTATTAACGTGGTATACAGTCGTGGCAAAAGTATGGGCGTCTT
ATTTGGCGGACGTAGTTACATGCCATCCACACATAGGACTACCGAAAAGTGGAACAGT
GTAGCAGACTGTCTACCTTGCGTATTTTTAGTCGATTTTGAATTCGGATGCGCTACAT
CATACATTTTGCCAGAATTGCAAGACGGTCTAAGTTTTCATGTGTCAATTGCAAAAAA
TGATACAATTTACATTTTAGGTGGTCATTCCTTAGCAAATAATATCAGACCAGCCAAT
TTGTACCGTATTAGGGTCGATTTACCATTGGGATCACCCGCCGTGAATTGCACTGTCT
TACCAGGAGGTATCTCTGTATCTTCAGCCATCTTAACTCAGACTAATAATGATGAATT
CGTGATAGTAGGTGGCTATCAATTAGAAAACCAAAAGAGAATGATTTGTAACATCATT
TCCTTGGAGGACAACAAGATTGAAATTAGAGAAATGGAAACCCCAGATTGGACACCCG
ATATAAAACACTCAAAGATTTGGTTCGGATCCAACATGGGTAATGGAACTGTTTTCTT
GGGAATTCCAGGCGACAATAAACAAGTGGTTTCTGAAGGTTTTTATTTCTACATGCTA
AAGTGCGCAGAAGATGATACCAACGAAGAACAAACTACATTCACAAATTCACAGACCT
CAACCGAAGACCCAGGCGACTCTACTCCTTTTGAAGACTCAGAAGAGTTTTGCTTTAG
TGCAGAGGCCAATTCCTTTGACGGTGATGACGAGTTCGATACATACAATGAAGACGAT
GAAGAAGACGAGTCCGAAACTGGCTATTGGATAACCTGTTGTCCTACTTGTGATGTTG
ACATTAATACCTGGGTTCCCTTCTATTCAACTGAGCTAAATAAGCCAGCCATGATTTA
TTGCTCTCATGGCGATGGTCATTGGGTACATGCTCAATGTATGGACTTAGCAGAGAGG
ACCTTGATTCATTTGTCTGCCGGTAGTAACAAATATTACTGTAATGAACATGTCGAAA
TTGCAAGAGCATTACATACACCTCAGAGAGTTTTGCCTTTGAAGAAACCACCCATGAA
ATCCTTAAGAAAAAAGGGTAGTGGCAAAATTTTAACTCCTGCTAAAAAATCTTTTTTG
AGGCGTTTGTTTGATTAA
5.
HMGB1_human
SEQ ID NO: 5
ATGGGAAAAGGAGATCCAAAAAAGCCTAGAGGCAAGATGTCATCTTATGCATTTTTCG
TTCAAACATGCAGAGAAGAACACAAGAAAAAACACCCAGACGCCTCAGTGAATTTTTC
TGAATTCTCAAAAAAATGTTCAGAAAGGTGGAAAACTATGAGTGCCAAAGAAAAGGGT
AAATTTGAGGACATGGCAAAAGCCGATAAGGCAAGATACGAAAGAGAAATGAAAACTT
ACATTCCACCAAAGGGAGAAACCAAAAAGAAATTCAAGGACCCCAATGCACCTAAGAG
GCCCCCATCCGCATTTTTCTTGTTTTGTTCCGAATATCGTCCTAAAATAAAAGGTGAA
CATCCTGGTTTGTCTATAGGTGACGTTGCAAAGAAGTTAGGAGAAATGTGGAACAACA
CTGCTGCTGATGATAAACAGCCTTACGAAAAGAAAGCAGCCAAACTAAAAGAAAAATA
TGAGAAGGATATCGCAGCATACAGGGCTAAAGGTAAACCCGATGCTGCAAAGAAAGGA
GTAGTCAAGGCTGAAAAATCCAAGAAAAAGAAGGAGGAGGAAGAAGATGAAGAGGATG
AGGAGGATGAGGAAGAGGAAGAAGATGAAGAAGATGAGGACGAAGAGGAGGATGATGA
TGACGAATAA
6.
12-RSS
SEQ ID NO: 6
CACAGTGCTACAGACTGGAACAAAAACC
7.
23-RSS
SEQ ID NO: 7
CACAGTGGTAGTACTCCACTGTCTGGCTGTACAAAAACC
8.
12-RSS-mut
SEQ ID NO: 8
GTACAGCCTACAGACTGGACAGTCAGTA
9.
23-RSS-mut
SEQ ID NO: 9
GTACAGCGTAGTACTCCACTGTCTGGCTGTCAGTCAGTA
10.
23-RSS-2 (used in split eGFP plasmids)
SEQ ID NO: 10
CACAGTGTGAAAACCCACATCCTGAGAGTGACAAAAACC
11.
23-RSS-3 (used in split eGFP/scFv plasmids)
SEQ ID NO: 11
CACAGTGAGGGGAAGTCATTGTGAGCCCAGACAAAAACC
12.
HMGB1 Consensus Identity Sequence
SEQ ID NO: 12
MGKGDPKKPRGKMSSYAFFVDTCREEHKKKHPDASVNFSEFSKKCSERWKTMSAKEKG
KFEDMAKADKARYEREMKTYIPPKHETKKKFKDPNAPKRPPSAFFLFCSEYRPKIKGE
HPGLSIGDVAKKLGEMWNNTAADDKQPYEKKAKLKEKYEKDIAAYRAKGKPDAAKKGV
VKAEKSKKKKEEEXDEEDEEDEEEEEXEEDEDEEEDDDDE
wherein X refers to either aspartate (D) or glutamate (E).
13.
HMGB1_HUMAN
SEQ ID NO: 13
MGKGDPKKPRGKMSSYAFFVQTCREEHKKKHPDASVNFSEFSKKCSERWKTMSAKEKG
KFEDMAKADKARYEREMKTYIPPKGETKKKFKDPNAPKRPPSAFFLFCSEYRPKIKGE
HPGLSIGDVAKKLGEMWNNTAADDKQPYEKKAAKLKEKYEKDIAAYRAKGKPDAAKKG
VVKAEKSKKKKEEEEDEEDEEDEEEEEDEEDEDEEEDDDDE
14.
HMGB1_MOUSE
SEQ ID NO: 14
MGKGDPKKPRGKMSSYAFFVQTCREEHKKKHPDASVNFSEFSKKCSERWKTMSAKEKG
KFEDMAKADKARYEREMKTYIPPKGETKKKFKDPNAPKRPPSAFFLFCSEYRPKIKGE
HPGLSIGDVAKKLGEMWNNTAADDKQPYEKKAAKLKEKYEKDIAAYRAKGKPDAAKKG
VVKAEKSKKKKEEEDEEDEEDEEEEEEEEDEDEEEDDDDE
15.
Representative sequencing of homology-assisted coding joint
(5′ → 3′)
SEQ ID NO: 15
TGCATTCGATTCCTGTTTGTAATTGTCCTTTTAACAGCGATCGCGTATTTCGTCTCGC
TC
16.
Representative sequencing of homology-assisted coding joint
(5′ → 3′)
SEQ ID NO: 16
ACGTAAGCTAAGGACAAACATTAACAGGAAAATTGTCGCTAGCGCATAAAGCAGAGCG
AG
17.
Representative sequencing of integrated signal joint formation assay
(5′ → 3′)
SEQ ID NO: 17
GAAGACCTGTGCGGTTTTTGTTCCAGTCTGTAGCACTGTGCACAGTGGTAGTACTCCA
CTGTCTGGCTGTACAAAAACCCCTCAGGTCTTC
18.
Representative sequencing of integrated signal joint formation assay
(3′ → 5′)
SEQ ID NO: 18
CTTCTGGACACGCCAAAAACAAGGTCAGACATCGTGACACGTGTCACCATCATGAGGT
GACAGACCGACATGTTTTTGGGGAGTCCAGAAG
19.
Representative sequencing of a fluorescent, AC518-SJ colony grown
on a 5-FOA plate (5′ → 3′)
SEQ ID NO: 19
GAAGACCTGTGCGGTTTTTGTTCCAGTCTGTAGCACTGTGCACGGTGCAACACTACTT
CAACTTCATCTTACAAAAAGATCACGTGATCTG
20.
Representative sequencing of a fluorescent, AC518-SJ colony grown
on a 5-FOA plate (3′ → 5′)
SEQ ID NO: 20
CTTCTGGACACGCCAAAAACAAGGTCAGACATCGTGACACGTGCCACGTTGTGATGAA
GTTGAAGTAGAATGTTTTTCTAGTGCACTAGAC
21.
G418R Template
SEQ ID NO: 21
GCGCTGGCAGTGTTCCTGCGCCGGTTGCATTCGATTCCTGTTTGT
22.
Representative sequencing of V(D)J-created true
coding joint (5′ → 3′)
SEQ ID NO: 22
GCGCTGGCAGTGTTCCTGCGCGCGCGGTTGCATTCGATTCCTGTTTGTAATTGT
23.
Representative sequencing of V(D)J-created true
coding joint ((5′ → 3′).
SEQ ID NO: 23
AGGTGAAAATATTGTTGATGCGCTGGCAGTGTTCCTGCGCGCGCGGTTGCATTCGATT
CCTGTTTGTAATTGTCCTTTTAACAGCGAT
24.
Representative sequencing of V(D)J-created true
coding joint (5′ → 3′)
SEQ ID NO: 24
GCGCTGGCAGTGTTCCTGCGCGCGTTGCATTCGATTCCTGTTTGTAATTGT
25.
Representative sequencing of V(D)J-created true
coding joint (5′ → 3′)
SEQ ID NO: 25
AGGTGAAAATATTGTTGATGCGCTGGCAGTGTTCCTGCGCGCGTTGCATTCGATTCCT
GTTTGTAATTGTCCTTTTAACAGCGAT
26.
Representative sequencing of V(D)J-created true
coding joint (5′ → 3′)
SEQ ID NO: 26
GCGCTGGCAGTGTTCCTGCGCGCGCAGTTGCATTCGATTCCTGTTTGTAATTGT
27.
Representative sequencing of V(D)J-created true
coding joint ((5′ → 3′)
SEQ ID NO: 27
AGGTGAAAATATTGTTGATGCGCTGGCAGTGTTCCTGCGCGCGCAGTTGCATTCGATT
CCTGTTTGTAATTGTCCTTTTAACAGCGAT
28.
G418R Template
SEQ ID NO: 28
ALAVFLRRLHSIPVC
29.
VDJ C1
SEQ ID NO: 29
ALAVFLRARLHSIPVC
30.
VDJ C3, VDJ C5, and VDJ C6
SEQ ID NO: 30
ALAVFLRALHSIPVC
31.
VDJ C4
SEQ ID NO: 31
ALAVFLRAQLHSIPVC
32.
KanR
SEQ ID NO: 32
AGGTGAAAATATTGTTGATGCGCTGGCAGTGTTCCTGCGCCGGTTGCATTCGATTCCT
GTTTGTAATTGTCCTTTTAACAGCGAT
33.
KanR
SEQ ID NO: 33
GENIVDALAVFLRRLHSIPVCNCPFNSD
34
VDJ C1
SEQ ID NO: 34
GENIVDALAVFLRARLHSIPVCNCPFNSD
35
VDJ C3, VDJ C5, and VDJ C6
SEQ ID NO: 35
GENIVDALAVFLRALHSIPVCNCPFNSD
36.
VDJ C4
SEQ ID NO: 36
GENIVDALAVFLRAQLHSIPVCNCPFNSD
37.
12-RSS-heptamer-region
SEQ ID NO: 37
TTGT'CACAGTG
Apostrophe represents cut site; underlined nucleotides
represent RSS heptamer
38.
23-RSS-heptamer-region
SEQ ID NO: 38
CACTGTG'AATT
Apostrophe represents cut site; underlined nucleotides
represent RSS heptamer
39.
12-RSS-heptamer-region-modified
SEQ ID NO: 39
GCGC'CACAGTG
Apostrophe represents cut site; underlined nucleotides
represent RSS heptamer
40.
23-RSS-heptamer-region-modified
SEQ ID NO: 40
CACTGTG'CGGT
Apostrophe represents cut site; underlined nucleotides
represent RSS heptamer
41.
12-RSS H0opt mutant
SEQ ID NO: 41
GCGCCACAGTGCTACAGACTGGAACAAAAACC
42.
23-RSS H0opt mutant
SEQ ID NO: 42
ACCGCACAGTGGTAGTACTCCACTGTCTGGCTGTACAAAAACC
43.
Sequence of the 5′ region of the G418 resistance gene to be
recombined via V(D)J in the pY112-CJA-U-H0opt plasmid
SEQ ID NO: 43
AGGTGAAAATATTGTTGATGCGCTGGCAGTGTTCCTGCGC
44.
Sequence of the 3′ region of the G418 resistance gene to be
SEQ ID NO: 44
recombined via V(D)J in the pY112-CJA-U-H0opt plasmid
CGGTTGCATTCGATTCCTGTTTGTAATTGTCCTTTTAACAGCGAT
45.
MRE11
SEQ ID NO: 45
MDYPDPDTIRILITTDNHVGYNENDPITGDDSWKTFHEVMMLAKNNNVDMVVQSGDLF
HVNKPSKKSLYQVLKTLRLCCMGDKPCELELLSDPSQVFHYDEFTNVNYEDPNFNISI
PVFGISGNHDDASGDSLLCPMDILHATGLINHFGKVIESDKIKVVPLLFQKGSTKLAL
YGLAAVRDERLFRTFKDGGVTFEVPTMREGEWFNLMCVHQNHTGHTNTAFLPEQFLPD
FLDMVIWGHEHECIPNLVHNPIKNFDVLQPGSSVATSLCEAEAQPKYVFILDIKYGEA
PKMTPIPLETIRTFKMKSISLQDVPHLRPHDKDATSKYLIEQVEEMIRDANEETKQKL
ADDGEGDMVAELPKPLIRLRVDYSAPSNTQSPIDYQVENPRRFSNRFVGRVANGNNVV
QFYKKRSPVTRSKKSGINGTSISDRDVEKLFSESGGELEVQTLVNDLLNKMQLSLLPE
VGLNEAVKKFVDKDEKTALKEFISHEISNEVGILSTNEEFLRTDDAEEMKALIKQVKR
ANSVRPTPPKENDETNFAFNGNGLDSFRSSNREVRTGSPDITQSHVDNESRITHISQA
ESSKPTSKPKRVRTATKKKIPAFSDSTVISDAENELGDNNDAQDDVDIDENDIIMVST
DEEDASYGLLNGRKTKTKTRPAASTKTASRRGKGRASRTPKTDILGSLLAKKRK
46.
RAD50
SEQ ID NO: 46
MSAIYKLSIQGIRSFDSNDRETIEFGKPLTLIVGMNGSGKTTIIECLKYATTGDLPPN
SKGGVFIHDPKITGEKDIRAQVKLAFTSANGLNMIVTRNIQLLMKKTTTTFKTLEGQL
VAINNSGDRSTLSTRSLELDAQVPLYLGVPKAILEYVIFCHQEDSLWPLSEPSNLKKK
FDEIFQAMKFTKALDNLKSIKKDMSVDIKLLKQSVEHLKLDKDRSKAMKLNIHQLQTK
IDQYNEEVSEIESQLNEITEKSDKLFKSNQDFQKILSKVENLKNTKLSISDQVKRLSN
SIDILDLSKPDLQNLLANFSKVLMDKNNQLRDLETDISSLKDRQSSLQSLSNSLIRRQ
GELEAGKETYEKNRNHLSSLKEAFQHKFQGLSNIENSDMAQVNHEMSQFKAFISQDLT
DTIDQFAKDIQLKETNLSDLIKSITVDSQNLEYNKKDRSKLIHDSEELAEKLKSFKSL
STQDSLNHELENLKTYKEKLQSWESENIIPKLNQKIEEKNNEMIILENQIEKFQDRIM
KTNQQADLYAKLGLIKKSINTKLDELQKITEKLQNDSRIRQVFPLTQEFQRADLEMDF
QKLFINMQKNIAINNKKMHELDRRYTNALYNLNTIEKDLQDNQKSKEKVIQLLSENLP
EDCTIDEYNDVLEETELSYKTALENLKMHQTTLEFNRKALEIAERDSCCYLCSRKFEN
ESFKSKLLQELKTKTDANFEKTLKDTVQNEKEYLHSLRLLEKHIITLNSINEKIDNSQ
KCLEKAKEETKTSKSKLDELEVDSTKLKDEKELAESEIRPLIEKFTYLEKELKDLENS
SKTISEELSIYNTSEDGIQTVDELRDQQRKMNDSLRELRKTISDLQMEKDEKVRENSR
MINLIKEKELTVSEIESSLTQKQNIDDSIRSKRENINDIDSRVKELEARIISLKNKKD
EAQSVLDKVKNERDIQVRNKQKTVADINRLIDRFQTIYNEVVDFEAKGFDELQTTIKE
LELNKAQMLELKEQLDLKSNEVNEEKRKLADSNNEEKNLKQNLELIELKSQLQHIESE
ISRLDVQNAEAERDKYQEESLRLRTRFEKLSSENAGKLGEMKQLQNQIDSLTHQLRTD
YKDIEKNYHKEWVELQTRSFVTDDIDVYSKALDSAIMKYHGLKMQDINRIIDELWKRT
YSGTDIDTIKIRSDEVSSTVKGKSYNYRVVMYKQDVELDMRGRCSAGQKVLASIIIRL
ALSETFGANCGVIALDEPTTNLDEENIESLAKSLHNIINMRRHQKNFQLIVITHDEKF
LGHMNAAAFTDHFFKVKRDDRQKSQIEWVDINRVTY
47.
XRS2
SEQ ID NO: 47
MWVVRYQNTLEDGSISFISCCLQAFKTYSIGRSSKNPLIIKNDKSISRQHITFKWEIN
NSSDLKHSSLCLVNKGKLTSLNKKFMKVGETFTINASDVLKSTIIELGTTPIRIEFEW
INEVWNIPPHLTQFRTMLSEYGISTEISINDIPANLMISDYPKSEDNSIRELYALVST
IPMKKSRFLMELCNTLLPTSKTNLKFDEMWNDMISNPEYNVFDFDPNILLSKFMRLNN
IRVLTTIKSEPRLSSLLRTFNINLFAFDNIDSLYKYVDSLEASTEYLILTTTDKKENG
KILCTIKTMLTSIIDGTLSAVINMKGASSRTLDNGKFDQISEGMSTILKTSRAPEVEA
SPVVSKKRKLNRRRVLPLDSLDFFAGGLSTKTLSENRSLTDAKRLNCGAESKTVISSP
NIAEADEKHAPFLQNALKPTEDIGKKSGHSSPGAIIVSSPNLGTVNTSEDSLDKSLQS
HKLPQPSLPEVAGIGSQTISSNSADYETAAVNSMDDAEVTKNFRVNHHQNIEQPSKNI
RKLSNYSREISSPLQENCKSPVKELSIKEKSGTPHAFVEAIQETKNREVKRVKSTIVE
LKDEELSEEAINQLKNLAIVEPSNNLLRKSFDSEGNKTSRTTEKWENSLMEPEWHKRK
NFKTFVKVRPKSKAHKEEGKNNTQSSDFIRNAAFLITRNYVPLKKYSKKDTTTKWGTE
ENEDMFALTEMERFGSNTFMSDNINSNTIQKRSQALNRFTNEDSSNEGEEDSFSFSRC
SGTAASVQPLKNKIFITDEDDLGDIDDKSDRLNHRENNRNLFVVKEMNLRPNLSEECS
KQSRHSRSATSRSRGSFGASNNGDGDDDDDDGPKFTFKRRKG
48.
pY112-CJA-U-H0opt
SEQ ID NO: 48
GAGGTCGACACTGGATGGCGGCGTTAGTATCGAATCGACAGCAGTATAGCGACCAGCA
TTCACATACGATTGACGCATGATATTACTTTCTGCGCACTTAACTTCGCATCTGGGCA
GATGATGTCGAGGCGAAAAAAAATATAAATCACGCTAACATTTGATTAAAATAGAACA
ACTACAATATAAAAAAACTATACAAATGACAAGTTCTTGAAAACAAGAATCTTTTTAT
TGTCAGTACTGTTTAAGCCAAGATTTCCTTGACAGCCTTGGCGATAGCATCGCCAACC
TCAGTGGTAGAGTTAGAACCACCAAGGTCACCGGTTCTGACACCTGCATCCAAGACAT
TTCTAACAGCTTCTTCAAGAGCCCTACCTTCTTCAACCAAATCCAAGGATAACTTCAA
CATCATAGCTGCAGATAAGATGGTAGCAATTGGGTTAACCTTGTTTGCTGGTAAATCT
GGGGCAGAACCATGACATGGTTCGTACAAACCGAATGCCTTGTTAGTGTCAGGTAGGG
AAGCTAGAGATGCAGAAGGTAATAAACCCAAAGAACCTGGAATAACAGAGGCTTCATC
GGAGATAATATCACCAAACATGTTGTTGGTAATAACAACACCGTTTAGCTTAGTTGGT
GATTTAACCAAAATCATAGCAGCAGAATCGATCAATTGGTGCTGAACAGTTAATTGTG
GGAACTCAGTCTTGATGGTTTCTTCAACAGTCTTTCTCCACAATCTGGAAGAGGCAAG
CACGTTAGCCTTGTCAAGTGACCAGATTGGTAATGGTGGGTTTTGTTGCAATGCCAAG
AAAGCAGCCATTCTTGTAATTCTTTGAACTTCAGGAACACTGTATTTCTCAGAATCCC
AAGCAACTCCGTCACCTTCATCTTCTTTTCTTTCACCAAAGTAGATACCACCAACCAA
TTCTCTAACGACGACGAAATCGGTACCCTTTGCATATTCAGGCTTCAAAGGAGAAAGA
TCTAGTAAAGAATCAGAAGCAAAGTTACATGGCCTCAAGTTGGCGTATAGACCCAATT
CCTTTCTGATCTTCAATAGACCTTGTTCTGGTCTAACTGCGCCCGTACCCCATTTTGG
ACCACCAACAGCACCTAGTAAGACAGCATCGGCTTTCTTAGAGGCTTCTAGAGCTTCA
TCTGGTAAAGGAGTGCCAGTGGCATCGATGGCAGCACCCCCGATCAAGTGATGTTGGA
AATTGAACTTAATTTCTGGACGGACTTCAGCAATGGCATTCAAGACCTTAATAGCTTC
GTCAGTAACTTCTTTACCGACGTGATCACCCGGTAGGACAACGATATTCTTAGACATT
GTTGTTTATGTTCGGATGTGATGTGAGAACTGTATCCTAGCAAGATTTTAAAAGGAAG
TATATGAAAGAAGAACCTCAGTGGCAAATCCTAACCTTTTATATTTCTCTACAGGGGC
GCGGCGTGGGGACAATTCAACGCGTCTGTGAGGGGAGCGTTTCCCTGCTCGCAGGTCT
GCAGCGAGGAGCCGTAATTTTTGCTTCGCGCCGTGCGGCCATCAAAATGTATGGATGC
AAATGATTATACATGGGGATGTATGGGCTAAATGTACGGGCGACAGTCACATCATGCC
CCTGAGCTGCGCACGTCAAGACTGTCAAGGAGGGTATTCTGGGCCTCCATGTCGCTGG
CCGGGTGACCCGGCGGGGACAAGGCAAGCTGCACCTAGATCCTTTTAAATTAAAAATG
AAGTTTTAAATCAATCTAAAGTATATATGAGTAAACTTGGTCTGACAGAGTTCTGAGG
TCATTACTGGATCTATCAACAGCAGTCCAAGCGAGCTCGATATCAAATTACGCCCCGC
CCTGCCACTCATCGCAGTACTGTTGTAATTCATTAAGCATTCTGCCGACATGGAAGCC
ATCACAAACGGCATGATGAACCTGAATCGCCAGCGGCATCAGCACCTTGTCGCCTTGC
GTATAATATTTGCCCATGGTGAAAACGGGGGCGAAGAAGTTGTCCATATTGGCCACGT
TTAAATCAAAACTGGTGAAACTCACCCAGGGATTGGCTGAGACGAAAAACATATTCTC
AATAAACCCTTTAGGGAAATAGGCCAGGTTTTCACCGTAACACGCCACATCTTGCGAA
TATATGTGTAGAAACTGCCGGAAATCGTCGTGGTATTCACTCCAGAGCGATGAAAACG
TTTCAGTTTGCTCATGGAAAACGGTGTAACAAGGGTGAACACTATCCCATATCACCAG
CTCACCGTCTTTCATTGCCATACGAAATTCCGGATGAGCATTCATCAGGCGGGCAAGA
ATGTGAATAAAGGCCGGATAAAACTTGTGCTTATTTTTCTTTACGGTCTTTAAAAAGG
CCGTAATATCCAGCTGAACGGTCTGGTTATAGGTACATTGAGCAACTGACTGAAATGC
CTCAAAATGTTCTTTACGATGCCATTGGGATATATCAACGGTGGTATATCCAGTGATT
TTTTTCTCCATTTTAGCTTCCTTAGCTCCTGAAAATCTCGATAACTCAAAAAATACGC
CCGGTAGTGATCTTATTTCATTATGGTGAAAGTTGGAACCTCTTACGTGCCCGATCAA
CTCGCGCGTTTGCCACCTGACGTCTAAGAAAAGGAATATTCAGCAATTTGCCCGTGCC
GAAGAAAGGCCCACCCGTGAAGGTGAGCCAGTGAGTTGATTGGAAGACCTGTGCGTAA
TGCTTCAAAAACGCACTGTACTCCTTTTTACTCTTCCGGATTTTCTCGCACTCTCCGC
ATCGCCGCACGAGCCAAGCCACACCCACACACCTCATACCATGTTTCCCCTCTTTGTC
TCTTTCGTGCGGCTCCATTACCCGCATGAAACTGTATAAAAGTAACAAAAGACTATTT
CGTTTCTTTTTCTTTGTCGGAAAAGGCAAAAAAAAAAATTTTTATCACATTTCTTTTT
CTTGAAAATTTTTTTTGGGATTTTTTCTCTTTCGATGACCTCCCATTGATATTTAAGT
TAATAAAAGCACTCCCGTTTTCCAAGTTTTAATTTGTTCCTCTTGTTTAGTCATTCTT
CTTCTCAGCATTGGTCAATTAGAAAGAGAGCATAGCAAACTGATCTAAGTTTTAATTA
CAAAATGGGTAAGGAAAAGACACACGTTTCGAGGCCGCGATTAAATTCCAACATGGAT
GCTGATTTATATGGGTATAAATGGGCTCGCGATAATGTCGGGCAATCAGGTGCGACAA
TCTATCGATTGTATGGGAAGCCCGATGCGCCAGAGTTGTTTCTGAAACATGGCAAAGG
TAGCGTTGCCAATGATGTTACAGATGAGATGGTCAGACTAAACTGGCTGACGGAATTT
ATGCCTCTTCCGACCATCAAGCATTTTATCCGTACTCCTGATGATGCATGGTTACTCA
CCACTGCGATCCCCGGCAAAACAGCATTCCAGGTATTAGAAGAATATCCTGATTCAGG
TGAAAATATTGTTGATGCGCTGGCAGTGTTCCTGCGCCACAGTGCTACAGACTGGAAC
AAAAACCCAATACAACAGATCACGTGATCTTTTTGTAAGATGAAGTTGAAGTAGTGTT
GCACCGTGCCAATGCAGGTGGCTATTAGATTAAATATGTGATTTGTTCTATTAAGTTT
CCTGTATAATTTAATGGGGAGCGCTGATTCTCTTTTGGTACGCTTCCCATCCAGCATT
TCTGTATCTTTCACCTTCAACCTTAGGATCTCTACCCTTGGCGAAAAGTCCTCTGCCA
ACAATGATGATATCTGATCCACCACTTACAACTTCGTCGACGGTTCTGTACTGCTGAC
CCAATGCATCGCCTTTGTCGTCTAAACCTACACCTGGGGTCATGATTAGCCAATCAAA
CCCTTCTTCTCTTCCTCCCATATCGTTCTGAGCAATGAACCCAATAACGAAATCTTTA
TCACTCTTTGCAATATCAACGGTACCCTTAGTATATTCACCGTGTGCTAGAGAACCCT
TGGATGACAATTCAGCAAGCATCAATAATCCCCTTGGTTCTTTGGTGACCTCTTGCGC
ACCTTGTTTCAAGCCAGCAACAATACCAGCACCAGTAACCCCGTGGGCGTTGGTGATA
TCAGACCATTCTGCGATACGGTAAACGCCCGATGTATATTGTAATTTGACTGTGTTAC
CGATATCGGCGAATTTTCTGTCCTCAAATATCAAGAACTTGTATTTCTCTGCCAATGC
TTTCAATGGAACGACAGTACCCTCATAACTGAAATCATCCAAGATATCAACGTGTGTT
TTCAAAAGGCAAATGTATGGACCCAACGTTTCAACAAGTTTCAATAGCTCATCAGTCG
AACGAACGTCAAGAGAAGCACACAAATTGGTTTTCTTTTCATCCATTAAACGTAAAAG
TTTCGATGCAACCGGACTTGCATGTGTCTCAGCTCTACTGGTATATGATTTTGTGGAC
ATTTTGAATATGTATTACTTGGTTATGGTTATATATGACAAAAGAAAAAGAAGAACAG
AAGAATAACGCAAGGAAGAACAATAACTGAAATTGATAGAGAAGTATTATGTCTTTGT
CTTTTTATAATAAATCAAGTGCAGAAATCCGTTAGACAACATGAGGGATAAAATTTAA
CGTGGGCGAAGAAGAAGGAAAAAAGTTTTTGTGAGGGCGTAATTGAAGCGATCTGTTG
ATTGTAGATTTTTTTTTTTTGAGGAGACAAAGTCAGAAGAGAACAGACAAATGGTATT
AACCATCCAATACTTTTTTGGAGCAACGCTAAGCTCATGCTTTTCCATTGGTTACGTG
CTCAGTTGTTAGATATGGAAAGAGAGGATGCTCACGGCAGCGTGACACCAATTGAGCC
CGAAAGAGAGGATGCCACGTTTTCCCGACGGCTGCTAGAATGGAAAAAGGAAAAATAG
AAGAATCCCATTCCTATCATTATTTACGTAATGACCCACACATTTTTGAGATTTTCAA
CTATTACGTATTACGATAATCCTGCTGTCATGGTTTTTGTACAGCCAGACAGTGGAGT
ACTACCACTGTGCGGTTGCATTCGATTCCTGTTTGTAATTGTCCTTTTAACAGCGATC
GCGTATTTCGTCTCGCTCAGGCGCAATCACGAATGAATAACGGTTTGGTTGATGCGAG
TGATTTTGATGACGAGCGTAATGGCTGGCCTGTTGAACAAGTCTGGAAAGAAATGCAT
AAGCTTTTGCCATTCTCACCGGATTCAGTCGTCACTCATGGTGATTTCTCACTTGATA
ACCTTATTTTTGACGAGGGGAAATTAATAGGTTGTATTGATGTTGGACGTGTCGGAAT
CGCAGACCGATACCAGGATCTTGCCATCCTATGGAACTGCCTCGGTGAGTTTTCTCCT
TCATTACAGAAACGGCTTTTTCAAAAATATGGTATTGATAATCCTGATATGAATAAAT
TGCAGTTTCATTTGATGCTCGATGAGTTTTTCTAAATTTGATCTGTAGCCTAAGTATA
AAATTCTACGTATGTATATATTTACATGCAATTTTTTCTTTTTCCAATTCATGGTACC
TCAGGTCTTCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCAGGAACCG
TAAAAAGGCCGCGTTGCTGGCGTTTTTCCATAGGCTCCGCCCCCCTGACGAGCATCAC
AAAAATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAACCCC
CCGTTCAGCCCGACCGCTGCGCCTTATCCGGTAACTATCGTCTTGAGCCCAACCCGGT
AAGACACGACTTATCGCCACTGGCAGCAGCCACTGGTAACAGGATTAGCAGAGCGAGG
TATGTAGGCGGTGCTACAGAGTTCTTGAAGTGGTGGCCTAACTACGGCTACACTAGAA
GAACAGTATTTGGTATCTGCGCTCTGCTGAAGCCAGTTACCTTCGGAAAAAGAGTTGG
TAGCTCTTGATCCGGCAAACAAACCACCGCTGGTAGCGGTGGTTTTTTTGTTTGCAAG
CAGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTATC
AAAAAGGATCTTCACGTGCGGACGGATCGCTTGCCTGTAACTTACACGCGCCTCGTAT
CTTTTAATGATGGAATAATTTGGGAATTTACTCTGTGTTTATTTATTTTTATGTTTTG
TATTTGGATTTTAGAAAGTAAATAAAGAAGGTAGAAGAGTTACGGAATGAAGAAAAAA
AAATAAACAAAGGTTTAAAAAATTTCAACAAAAAGCGTACTTTACATATATATTTATT
AGACAAGAAAAGCAGATTAAATAGATATACATTCGATTAACGATAAGTAAAATGTAAA
ATCACAGGATTTTCGTGTGTGGTCATCTACACAGACAAGATGAAACAATTCGGCATTA
ATACCTGAGAGCAGGAAGAGCAAGATAAAAGGTAGTATTTGTTGGCGATCCCCCTAGA
GTCTTTTACATCTTCGGAAAACAAAAACTATTTTTTCTTTAATTTCTTTTTTTACTTT
CTATTTTTAATTTATATATTTATATTAAAAAATTTAAATTATAATTATTTTTATAGCA
CGTGATGAAAAGGACCCAGGTGGCACTTTTCGGGGAATG

What is claimed is:

1. An engineered non-vertebrate cell comprising a nucleic acid encoding one or more recombination activating gene (RAG) proteins selected from RAG1, RAG2, or a variant thereof and a recombination signal sequence (RSS), wherein the engineered non-vertebrate cell is engineered to undergo variable-diversity-joining (V(D)J) recombination to generate an antibody or a fragment thereof.

2. The engineered non-vertebrate cell of claim 1, wherein the engineered non-vertebrate cell comprises a yeast cell.

3. The engineered non-vertebrate cell of claim 2, wherein the yeast cell is a Saccharomyces cerevisiae yeast cell.

4. The engineered non-vertebrate cell of claim 1, wherein the variant comprises a truncated RAG protein.

5. The engineered non-vertebrate cell of claim 4, wherein the truncated RAG protein comprises amino acids 348-1006, 383-1006, 284-1006, 215-1008, or 1-1008 of RAG1.

6. The engineered non-vertebrate cell of claim 4, wherein the truncated RAG protein comprises amino acids 1-383 of RAG2.

7. The engineered non-vertebrate cell of claim 1, wherein the nucleic acid encoding RAG1 comprises at least 70% sequence identity to SEQ ID NO: 1 or SEQ ID NO: 3.

8. The engineered non-vertebrate cell of claim 1, wherein the nucleic acid encoding RAG2 comprises at least 70% sequence identity to SEQ ID NO: 2 or SEQ ID NO: 4.

9. The engineered non-vertebrate cell of claim 1, wherein the RSS comprises a 12 base pair (bp) spacer or a 23 bp spacer.

10. The engineered non-vertebrate cell of claim 1, wherein the engineered non-vertebrate cell further comprises a nucleic acid encoding a High Mobility Group Box I (HMGB1) protein.

11. A method of generating an antibody or a fragment thereof, the method comprising:
a) obtaining a non-vertebrate cell,
b) introducing into the non-vertebrate cell a nucleic acid encoding one or more recombination activating gene (RAG) proteins selected from RAGI, RAG2, or variants thereof and a recombination signal sequence (RSS), and
c) culturing the non-vertebrate cell of step b) under cell culture conditions to allow V(D)J recombination activity,
wherein the non-vertebrate cell expresses an antibody or a fragment thereof following V(D)J recombination.

12. The method of claim 11, wherein the non-vertebrate cell comprises a yeast cell.

13. The method of claim 12, wherein the yeast cell is a Saccharomyces cerevisiae yeast cell.

14. The method of claim 11, wherein the nucleic acid encoding RAGI comprises at least 70% sequence identity to SEQ ID NO: 1 or SEQ ID NO: 3.

15. The method of claim 11, wherein the nucleic acid encoding RAG2 comprises at least 70% sequence identity to SEQ ID NO: 2 or SEQ ID NO: 4.

16. The method of claim 11, wherein the RSS comprises a 12 base pair (bp) spacer or a 23 bp spacer.

17. The method of claim 11, wherein the one or more RAG proteins bind to one RSS with a 12 bp spacer and binds to one RSS with a 23 bp spacer.

18. The method of claim 11, wherein the non-vertebrate cell further comprises a nucleic acid encoding a High Mobility Group Box I (HMGB1) protein.

19. The method of claim 11, wherein the V(D)J recombination activity occurs in vivo.

20. A method of screening for disease severity caused by a mutation to a recombination activating gene (RAG) using an engineered non-vertebrate cell, the method comprising:
a) obtaining a non-vertebrate cell,
b) engineering the non-vertebrate cell to express a mutant RAG protein, wherein the mutant RAG protein is associated with an immunodeficiency disease, and wherein the non-vertebrate cell comprises a recombination signal sequence,
c) culturing the non-vertebrate cell of step b) under cell culture conditions to allow V(D)J recombination activity, and
d) measuring V(D)J recombination activity in the non-vertebrate cell of step c), wherein a decrease in V(D)J recombination activity relative to a control non-vertebrate cell indicates an increase in severity of the immunodeficiency disease.


SEQUENCE LISTING (48 sequences)

SEQ ID NO: 1 - DNA, 3123 bp, Mus musculus
  source 1..3123: mol_type = genomic DNA
  misc_feature 1..3123: note = RAG1_mouse
atggctgcctccttgccgtctaccttgagcttcagttctgcacccgatgaaattcaacatccacaaatcaaattttccgagtggaaatttaagctgtttagggttagatcctttgaaaaggcacccgaagaagcacaaaaggaaaaagattcctcagaagggaaaccttacctagaacaatctccagtagttccagagaagcctggtggccaaaactcaattttgactcaacgtgcattgaaattgcatcctaaattttcaaagaaatttcatgctgatgggaagtcaagcgacaaagcagttcaccaagccaggttgagacatttctgtagaatctgtggtaatcgtttcaagagtgacggtcactcaagaagatacccagttcatggtcccgttgacgctaaaacccaaagtcttttccgtaaaaaggaaaaaagagtaacttcctggccagacttgattgccaggattttcagaatcgacgtaaaagcagatgttgactccattcacccgacggaattctgccatgattgttggagcatcatgcatagaaagttctctagttctcacagtcaggtttacttcccaaggaaagtaactgtggagtggcacccccacacaccgtcctgtgacatttgttttactgcacatagaggattgaagaggaagagacatcagcccaatgtacagctatctaagaaactaaaaactgtgttgaaccacgcgagaagggatcgtagaaagagaactcaagctagggtcagttcaaaggaagtcctaaagaagatctccaactgcagtaagattcatttgagtacaaagcttcttgccgtggacttcccagcacactttgtgaaatccatctcctgccaaatatgcgaacacattttagctgatcccgttgagacaagctgcaagcatctattctgtcgtatttgcattctgagatgtttaaaagttatgggctcttattgtccctcttgcaggtatccgtgcttccctactgacttagaaagcccagttaagtcctttttaaacattttgaattctctgatggttaagtgtcccgcgcaagattgcaatgaggaagtgagtttagaaaaatataaccaccatgtttcaagtcacaaagaatctaaagagactttggttcatatcaataaaggtggaagacctagacaacatctactgtcattaacgagaagggcgcagaaacatagattaagggagttaaagattcaagttaaagaatttgctgacaaagaagaaggtggggatgtaaaagctgtttgcttgacattgtttctactggcactgagggcgaggaatgaacacaggcaagctgatgaattagaggccataatgcaaggcaggggatccggtcttcaaccagctgtttgcttggctatccgtgtcaatacttttcttagctgttctcaatatcataagatgtataggactgttaaagctataactggtaggcaaatttttcaacctttgcatgctcttagaaatgccgaaaaagttcttttaccaggttaccatccatttgagtggcaacctccattgaagaatgtttcctctagaactgatgttggaataattgatggtctgtctggattggcatcttctgttgatgaatatccagtagatacgattgcgaaaaggttcagatacgattctgctttggtttctgctttgatggatatggaagaagacatcttggaaggcatgagatcccaagatcttgatgactacttgaatggtccatttacagttgttgtaaaggaatcttgcgatggaatgggagatgtgagtgaaaagcacggaagtgggcctgcagttccagaaaaggcggttcgtttctctttcacagttatgagaattacgatagaacatggttcacaaaacgtgaaggtttttgaggaacccaagcctaattctgaattatgttgcaagccgttgtgtcttatgttggcagatgagtctgaccatgagacacttactgctattctaagccccctaattgctgagagggaagccatgaagagtagtgaattaacgctagagatgggaggcatcccgaggacttttaaattcatattcaggggtactggatacgatgaaaaacttgtcagggaagtagaaggtttggaagcttctggctcagtatatatctgtacattgtgtgataccacccgtttggaagcctctcaaaatcttgtcttccactccataacgagaagccacgcagagaacctgcagagatatgaggtctggagatcaaatccgtatcatgagtccgtggaagagttgcgtgacagagtgaaaggtgtctctgccaaacctttcatcgaaacagttccttcaatagatgcgcttcactgtgatattggtaatgcagctgaattttataagattttccagctggagataggtgaagtatataaacatcctaatgcctctaaagaagaaagaaagagatggcaagccacgttagataaacatcttaggaaaagaatgaacttaaaaccaatcatgaggatgaatggtaactttgccagaaagttgatgactcaagagactgtagatgcagtttgtgaattaattccttctgaggagaggcatgaagctttgagggagttgatggacttatatctaaaaatgaaaccagtgtggagatcttcatgtccagctaaagaatgtccagagagtttgtgtcaatacagtttcaactcacagcgtttcgcggaactactgagtaccaagtttaaatatagatatgaaggtaaaataactaattactttcataaaaccttggcacatgtccctgaaattattgaaagggatggttctatcggggcatgggcaagtgaaggaaatgaaagcggtaacaaattgtttagaagatttaggaaaatgaatgccaggcagtctaagtgctatgagatggaagatgtcctgaaacatcactggttgtatacttcaaaatacttacagaagtttatgaatgctcataacgcgttaaaaagctctggatttaccatgaactcaaaggagacattaggggaccctttgggaattgaggattctctggaaagccaagattcaatggagttttaa

SEQ ID NO: 2 - DNA, 1584 bp, Mus musculus
  source 1..1584: mol_type = genomic DNA
  misc_feature 1..1584: note = RAG2_mouse
atgtccctgcagatggtaacagtgggtcataacatagcgttaattcaaccaggtttctcacttatgaattttgatggccaagttttcttctttggccaaaaaggctggccaaagcgttcctgtcctactggtgtctttcattttgatataaaacaaaatcatttaaaactgaagcctgcaatcttctctaaagattcctgctacctaccacctttgcgttacccagctacttgctcatacaaaggtagcatagactctgacaagcatcaatatatcattcacggtgggaagacaccgaacaatgagctatccgataagatttatatcatgtctgttgcttgcaagaataacaaaaaagttactttccgttgcacagagaaagacttagtaggtgatgtccctgaaccaagatacggccatagcattgacgtggtgtatagtagaggtaaaagcatgggtgttctgtttggaggtaggtcatacatgccttctacccaacgtaccacagaaaaatggaatagtgtagctgactgtctaccccatgttttcttgatagattttgaatttggttgtgctacatcatatattttgccagaacttcaagatgggctgtcttttcatgtttctattgccagaaacgatacggtttatattttggggggacactcattagccagtaatataagacctgctaacttgtatcgtataagagtggaccttccactgggtaccccagcagtgaattgtacagtcttgccaggaggaatctctgtatccagtgcgatcttaactcaaacaaacaatgatgaatttgttattgtgggtggttatcagctggaaaatcagaaaaggatggtctgtagcttggtttctctaggtgacaacacgattgaaatcagtgagatggagactccagactggacgtcagatattaagcatagcaaaatatggtttggaagcaacatgggaaacggtactattttccttggcataccaggagacaataagcaggctatgtcagaagcattctatttctatactttgagatgctctgaagaggatttgagtgaagatcagaaaattgtctccaacagtcaaacatcaacagaagatcctggtgactccactccctttgaagactcagaggaattttgtttcagtgctgaagcaaccagttttgatggtgacgatgaatttgacacctacaatgaagatgatgaagatgacgagtctgtaaccggctactggataacatgttgtccgacttgtgatgttgacatcaatacctgggttccgttctattcaacggagttaaataaaccagccatgatctattgttctcatggtgatgggcactgggtacatgcccaatgcatggatttggaagaaagaacattgatccacttgtcagaaggaagcaacaagtattattgcaatgaacatgtacaaatagcaagagcattgcaaactcccaaaagaaacccccccttacaaaaacctccaatgaaatccttgcacaaaaaaggctctggtaaagtcttgactcctgccaagaaatccttccttagaagactgtttgattaa

SEQ ID NO: 3 - DNA, 3132 bp, Homo sapiens
  source 1..3132: mol_type = genomic DNA
  misc_feature 1..3132: note = RAG1_human
atggctgcttcatttccacccacattgggtttatcaagtgctcccgacgaaattcaacacccacatataaaattttctgagtggaaattcaagttattcagggtgagatcctttgagaaaacacctgaggaagcacaaaaggaaaaaaaggacagtttcgaaggtaagccatctttagaacagtcacccgcagtcttagataaagcagatggccaaaaaccagttcccactcaaccattgctaaaggctcatccaaaatttagtaagaaattccacgacaacgaaaaagctaggggcaaagccatacaccaggccaatttaaggcatttgtgcagaatctgtggtaactcttttagagcagacgagcataaccgtagatatcccgtccacggccccgtagacggaaaaactttaggactattaaggaagaaggaaaaacgtgccacctcttggcctgatttaatcgctaaagtgttcaggattgacgtcaaagctgacgtagattccatccatcccactgagttctgtcataattgttggtcaataatgcatcgtaagttctcctctgccccctgtgaagtatatttcccaaggaacgtaacaatggaatggcacccacatacaccctcctgcgacatatgtaacaccgcaagaagaggattgaaaagaaaatcattgcaacccaatttacaactaagtaagaagttaaagaccgtattggatcaagccaggcaggctagacaaagaaagagacgtgcacaagcaaggatctcctctaaagacgtgatgaagaaaatagctaactgttcaaaaattcacttatcaaccaaattattagccgtcgatttcccagagcatttcgtcaaatcaatatcttgtcaaatttgtgaacacattttagctgatccagtagaaaccaattgtaaacatgtcttttgtagggtttgcattttgagatgcttaaaggttatgggttcttactgtccatcatgcagatacccatgcttccctactgatttggaaagtcctgtgaagagttttttgtctgtcttgaattccctaatggtcaaatgccctgcaaaggagtgtaatgaagaggtatcattagaaaaatataaccatcacatatcctcacataaagaaagtaaagagatcttcgtgcacattaataagggcggacgtccaaggcagcatttgttgtctttaactaggagagcacaaaaacacaggttgagagaattaaaattgcaagtcaaagctttcgctgacaaggaagagggcggtgatgttaagtcagtttgtatgacattattcttgttagcattgagagccagaaatgaacacaggcaggctgatgagttggaagccataatgcaaggaaaaggttctggcctacagcctgctgtctgcctagccatcagagtaaatactttcttgtcatgttcccaatatcacaagatgtatagaactgttaaagctatcacaggtaggcaaatttttcaaccattgcatgcattgaggaacgcagaaaaggtgttgttaccaggttaccaccactttgaatggcaacctcctttaaagaacgtatcttcctctaccgacgtgggtataatcgacggattgagtggcttgtcatcaagtgtggacgactatcctgtagatactattgccaaaaggtttagatatgattccgccttggtatctgcattgatggacatggaagaggatatcctagagggtatgaggtcacaggacttggatgattatttgaatggtcctttcacagtagtagttaaagaaagttgcgacggcatgggcgacgtatcagaaaaacatggttccggacctgttgtcccagaaaaggctgtgaggttctcttttacaatcatgaagataacaatagcacattctagtcaaaatgtaaaggtgtttgaagaagctaagccaaactccgaattatgttgcaagcctttatgcctaatgttagccgatgaaagtgatcacgaaacattaaccgctatcttatctccactaattgctgaaagggaagctatgaagtcatcagagttaatgttggagttgggtggtatcttgagaactttcaaattcatctttagaggaacaggttacgacgaaaagttggttagagaagtggaaggtttagaagcttccggatcagtatatatttgtactttgtgcgatgcaactagattagaagctagtcaaaacttagtctttcattcaataaccagatcacatgctgagaacttagagaggtatgaagtatggaggtccaacccctatcatgaatctgttgaagagttgagagatagagtcaagggtgttagtgcaaaacctttcatagaaaccgttccatccattgacgctttacactgtgacataggtaatgcagccgagttttacaagatattccagttagagataggtgaggtatataaaaatccaaacgcctctaaagaagaaagaaagagatggcaggccacactagataaacacctaagaaagaagatgaatttaaagcccatcatgaggatgaacggtaactttgcacgtaaactaatgacaaaggaaactgttgacgccgtatgcgaactaatcccttccgaagaaaggcatgaggcattgagagaattgatggacttgtatttgaaaatgaagcctgtctggcgtagttcttgccctgctaaggaatgtccagaatcactatgtcagtattcattcaattctcagagatttgcagagttgttatccacaaagttcaaatatcgttatgaaggaaagattacaaattattttcacaaaacattggcacatgtcccagaaataatagagagggatggttccataggagcatgggcctctgaaggaaacgaatcaggaaacaaactattccgtagattcagaaaaatgaacgcaagacagagtaagtgctatgagatggaagatgtgctaaagcatcactggctatatacatctaagtacctacagaagtttatgaacgcccacaatgcattgaaaacaagtggatttaccatgaatcctcaagcctccttgggagatccattgggtattgaggactccttagaatctcaagattccatggaattctaa

SEQ ID NO: 4 - DNA, 1584 bp, Homo sapiens
  source 1..1584: mol_type = genomic DNA
  misc_feature 1..1584: note = RAG2_human
atgagtctacagatggtcactgtctcaaacaatattgcattaattcagcctggattttctttaatgaatttcgatggtcaagtttttttcttcggccagaaaggttggcctaagagatcatgtcccaccggagtcttccatttagacgtcaagcataatcacgtaaagttgaagcccacaattttctccaaggatagttgctacctacctccactaagatatcctgccacatgcaccttcaaaggatcactagagtctgaaaaacaccaatacatcattcatggtggaaaaactccaaataatgaggtttccgataaaatttatgtaatgtccattgtttgcaaaaacaataagaaagttacttttagatgtaccgaaaaagatttggttggagacgtgccagaggcaagatacggtcatagtattaacgtggtatacagtcgtggcaaaagtatgggcgtcttatttggcggacgtagttacatgccatccacacataggactaccgaaaagtggaacagtgtagcagactgtctaccttgcgtatttttagtcgattttgaattcggatgcgctacatcatacattttgccagaattgcaagacggtctaagttttcatgtgtcaattgcaaaaaatgatacaatttacattttaggtggtcattccttagcaaataatatcagaccagccaatttgtaccgtattagggtcgatttaccattgggatcacccgccgtgaattgcactgtcttaccaggaggtatctctgtatcttcagccatcttaactcagactaataatgatgaattcgtgatagtaggtggctatcaattagaaaaccaaaagagaatgatttgtaacatcatttccttggaggacaacaagattgaaattagagaaatggaaaccccagattggacacccgatataaaacactcaaagatttggttcggatccaacatgggtaatggaactgttttcttgggaattccaggcgacaataaacaagtggtttctgaaggtttttatttctacatgctaaagtgcgcagaagatgataccaacgaagaacaaactacattcacaaattcacagacctcaaccgaagacccaggcgactctactccttttgaagactcagaagagttttgctttagtgcagaggccaattcctttgacggtgatgacgagttcgatacatacaatgaagacgatgaagaagacgagtccgaaactggctattggataacctgttgtcctacttgtgatgttgacattaatacctgggttcccttctattcaactgagctaaataagccagccatgatttattgctctcatggcgatggtcattgggtacatgctcaatgtatggacttagcagagaggaccttgattcatttgtctgccggtagtaacaaatattactgtaatgaacatgtcgaaattgcaagagcattacatacacctcagagagttttgcctttgaagaaaccacccatgaaatccttaagaaaaaagggtagtggcaaaattttaactcctgctaaaaaatcttttttgaggcgtttgtttgattaa

SEQ ID NO: 5 - DNA, 648 bp, Homo sapiens
  source 1..648: mol_type = genomic DNA
  misc_feature 1..648: note = HMGB1_human
atgggaaaaggagatccaaaaaagcctagaggcaagatgtcatcttatgcatttttcgttcaaacatgcagagaagaacacaagaaaaaacacccagacgcctcagtgaatttttctgaattctcaaaaaaatgttcagaaaggtggaaaactatgagtgccaaagaaaagggtaaatttgaggacatggcaaaagccgataaggcaagatacgaaagagaaatgaaaacttacattccaccaaagggagaaaccaaaaagaaattcaaggaccccaatgcacctaagaggcccccatccgcatttttcttgttttgttccgaatatcgtcctaaaataaaaggtgaacatcctggtttgtctataggtgacgttgcaaagaagttaggagaaatgtggaacaacactgctgctgatgataaacagccttacgaaaagaaagcagccaaactaaaagaaaaatatgagaaggatatcgcagcatacagggctaaaggtaaacccgatgctgcaaagaaaggagtagtcaaggctgaaaaatccaagaaaaagaaggaggaggaagaagatgaagaggatgaggaggatgaggaagaggaagaagatgaagaagatgaggacgaagaggaggatgatgatgacgaataa

SEQ ID NO: 6 - DNA, 28 bp, synthetic construct
  source 1..28: mol_type = other DNA
  misc_feature 1..28: note = 12-RSS
cacagtgctacagactggaacaaaaacc

SEQ ID NO: 7 - DNA, 39 bp, synthetic construct
  source 1..39: mol_type = other DNA
  misc_feature 1..39: note = 23-RSS
cacagtggtagtactccactgtctggctgtacaaaaacc

SEQ ID NO: 8 - DNA, 28 bp, synthetic construct
  source 1..28: mol_type = other DNA
  misc_feature 1..28: note = 12-RSS-mut
gtacagcctacagactggacagtcagta

SEQ ID NO: 9 - DNA, 39 bp, synthetic construct
  source 1..39: mol_type = other DNA
  misc_feature 1..39: note = 23-RSS-mut
gtacagcgtagtactccactgtctggctgtcagtcagta

SEQ ID NO: 10 - DNA, 39 bp, synthetic construct
  source 1..39: mol_type = other DNA
  misc_feature 1..39: note = 23-RSS-2 (used in split eGFP plasmids)
cacagtgtgaaaacccacatcctgagagtgacaaaaacc

SEQ ID NO: 11 - DNA, 39 bp, synthetic construct
  source 1..39: mol_type = other DNA
  misc_feature 1..39: note = 23-RSS-3 (used in split eGFP/scFv plasmids)
cacagtgaggggaagtcattgtgagcccagacaaaaacc

SEQ ID NO: 12 - protein, 214 aa, synthetic construct
  REGION 1..214: note = HMGB1 Consensus Identity Sequence; wherein X refers to either aspartate (D) or glutamate (E).
MGKGDPKKPRGKMSSYAFFVDTCREEHKKKHPDASVNFSEFSKKCSERWKTMSAKEKGKFEDMAKADKARYEREMKTYIPPKHETKKKFKDPNAPKRPPSAFFLFCSEYRPKIKGEHPGLSIGDVAKKLGEMWNNTAADDKQPYEKKAKLKEKYEKDIAAYRAKGKPDAAKKGVVKAEKSKKKKEEEXDEEDEEDEEEEEXEEDEDEEEDDDDE

SEQ ID NO: 13 - protein, 215 aa, Homo sapiens
  REGION 1..215: note = HMGB1_HUMAN
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 14 - protein, 214 aa, Mus musculus
  REGION 1..214: note = HMGB1_MOUSE
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEDEEDEEDEEEEEEEEDEDEEEDDDDE

SEQ ID NO: 15 - DNA, 60 bp, synthetic construct
  source 1..60: mol_type = other DNA
  misc_feature 1..60: note = Representative sequencing of homology-assisted coding joint (5' to 3')
tgcattcgattcctgtttgtaattgtccttttaacagcgatcgcgtatttcgtctcgctc

SEQ ID NO: 16 - DNA, 60 bp, synthetic construct
  source 1..60: mol_type = other DNA
  misc_feature 1..60: note = Representative sequencing of homology-assisted coding joint (3' to 5')
gagcgagacgaaatacgcgatcgctgttaaaaggacaattacaaacaggaatcgaatgca

SEQ ID NO: 17 - DNA, 91 bp, synthetic construct
  source 1..91: mol_type = other DNA
  misc_feature 1..91: note = Representative sequencing of integrated signal joint formation assay (5' to 3')
gaagacctgtgcggtttttgttccagtctgtagcactgtgcacagtggtagtactccactgtctggctgtacaaaaacccctcaggtcttc

SEQ ID NO: 18 - DNA, 91 bp, synthetic construct
  source 1..91: mol_type = other DNA
  misc_feature 1..91: note = Representative sequencing of integrated signal joint formation assay (3' to 5')
gaagacctgaggggtttttgtacagccagacagtggagtactaccactgtgcacagtgctacagactggaacaaaaaccgcacaggtcttc

SEQ ID NO: 19 - DNA, 91 bp, synthetic construct
  source 1..91: mol_type = other DNA
  misc_feature 1..91: note = Representative sequencing of a fluorescent, AC518-SJ colony grown on a 5-FOA plate (5' to 3')
gaagacctgtgcggtttttgttccagtctgtagcactgtgcacggtgcaacactacttcaacttcatcttacaaaaagatcacgtgatctg

SEQ ID NO: 20 - DNA, 91 bp, synthetic construct
  source 1..91: mol_type = other DNA
  misc_feature 1..91: note = Representative sequencing of a fluorescent, AC518-SJ colony grown on a 5-FOA plate (3' to 5')
cagatcacgtgatctttttgtaagatgaagttgaagtagtgttgcaccgtgcacagtgctacagactggaacaaaaaccgcacaggtcttc

SEQ ID NO: 21 - DNA, 45 bp, synthetic construct
  source 1..45: mol_type = other DNA
  misc_feature 1..45: note = G418R Template
gcgctggcagtgttcctgcgccggttgcattcgattcctgtttgt

SEQ ID NO: 22 - DNA, 54 bp, synthetic construct
  source 1..54: mol_type = other DNA
  misc_feature 1..54: note = Representative sequencing of V(D)J-created true coding joint (5` to 3`)
gcgctggcagtgttcctgcgcgcgcggttgcattcgattcctgtttgtaattgt

SEQ ID NO: 23 - DNA, 88 bp, synthetic construct
  source 1..88: mol_type = other DNA
  misc_feature 1..88: note = Representative sequencing of V(D)J-created true coding joint (5`to 3`)
aggtgaaaatattgttgatgcgctggcagtgttcctgcgcgcgcggttgcattcgattcctgtttgtaattgtccttttaacagcgat

SEQ ID NO: 24 - DNA, 51 bp, synthetic construct
  source 1..51: mol_type = other DNA
  misc_feature 1..51: note = Representative sequencing of V(D)J-created true coding joint (5` to 3`)
gcgctggcagtgttcctgcgcgcgttgcattcgattcctgtttgtaattgt

SEQ ID NO: 25 - DNA, 85 bp, synthetic construct
  source 1..85: mol_type = other DNA
  misc_feature 1..85: note = Representative sequencing of V(D)J-created true coding joint (5` to 3`)
aggtgaaaatattgttgatgcgctggcagtgttcctgcgcgcgttgcattcgattcctgtttgtaattgtccttttaacagcgat

SEQ ID NO: 26 - DNA, 54 bp, synthetic construct
  source 1..54: mol_type = other DNA
  misc_feature 1..54: note = Representative sequencing of V(D)J-created true coding joint (5` to 3`)
gcgctggcagtgttcctgcgcgcgcagttgcattcgattcctgtttgtaattgt

SEQ ID NO: 27 - DNA, 88 bp, synthetic construct
  source 1..88: mol_type = other DNA
  misc_feature 1..88: note = Representative sequencing of V(D)J-created true coding joint (5` to 3`)
aggtgaaaatattgttgatgcgctggcagtgttcctgcgcgcgcagttgcattcgattcctgtttgtaattgtccttttaacagcgat

SEQ ID NO: 28 - protein, 15 aa, synthetic construct
  REGION 1..15: note = G418R Template
ALAVFLRRLHSIPVC

SEQ ID NO: 29 - protein, 16 aa, synthetic construct
  REGION 1..16: note = VDJ C1
ALAVFLRARLHSIPVC

SEQ ID NO: 30 - protein, 15 aa, synthetic construct
  REGION 1..15: note = VDJ C3, VDJ C5, and VDJ C6
ALAVFLRALHSIPVC

SEQ ID NO: 31 - protein, 16 aa, synthetic construct
  REGION 1..16: note = VDJ C4
ALAVFLRAQLHSIPVC

SEQ ID NO: 32 - DNA, 85 bp, synthetic construct
  source 1..85: mol_type = other DNA
  misc_feature 1..85: note = KanR
aggtgaaaatattgttgatgcgctggcagtgttcctgcgccggttgcattcgattcctgtttgtaattgtccttttaacagcgat

SEQ ID NO: 33 - protein, 28 aa, synthetic construct
  REGION 1..28: note = KanR
GENIVDALAVFLRRLHSIPVCNCPFNSD

SEQ ID NO: 34 - protein, 29 aa, synthetic construct
  REGION 1..29: note = VDJ C1
GENIVDALAVFLRARLHSIPVCNCPFNSD

SEQ ID NO: 35 - protein, 28 aa, synthetic construct
  REGION 1..28: note = VDJ C3, VDJ C5, and VDJ C6
GENIVDALAVFLRALHSIPVCNCPFNSD

SEQ ID NO: 36 - protein, 29 aa, synthetic construct
  REGION 1..29: note = VDJ C4
GENIVDALAVFLRAQLHSIPVCNCPFNSD

SEQ ID NO: 37 - DNA, 11 bp, synthetic construct
  source 1..11: mol_type = other DNA
  misc_feature 1..11: note = 12-RSS
  misc_feature 4^5: note = Cut site
  misc_feature 5..11: note = RSS heptamer
ttgtcacagtg

SEQ ID NO: 38 - DNA, 11 bp, synthetic construct
  source 1..11: mol_type = other DNA
  misc_feature 7^8: note = cut site
  misc_feature 1..7: note = RSS heptamer
  misc_feature 1..11: note = 23-RSS
cactgtgaatt

SEQ ID NO: 39 - DNA, 11 bp, synthetic construct
  source 1..11: mol_type = other DNA
  misc_feature 1..11: note = 12-RSS
  misc_feature 4^5: note = cut site
  misc_feature 5..11: note = RSS heptamer
gcgccacagtg

SEQ ID NO: 40 - DNA, 11 bp, synthetic construct
  source 1..11: mol_type = other DNA
  misc_feature 1..11: note = 23-RSS
  misc_feature 7^8: note = cut site
  misc_feature 1..7: note = RSS heptamer
cactgtgcggt

SEQ ID NO: 41 - DNA, 32 bp, synthetic construct
  source 1..32: mol_type = other DNA
  misc_feature 1..32: note = 12-RSS H0opt mutant
gcgccacagtgctacagactggaacaaaaacc

SEQ ID NO: 42 - DNA, 43 bp, synthetic construct
  source 1..43: mol_type = other DNA
  misc_feature 1..43: note = 23-RSS H0opt mutant
accgcacagtggtagtactccactgtctggctgtacaaaaacc

SEQ ID NO: 43 - DNA, 40 bp, synthetic construct
  source 1..40: mol_type = other DNA
  misc_feature 1..40: note = Sequence of the 5' region of the G418 resistance gene to be recombined via V(D)J in the pY112-CJA-U-H0opt plasmid
aggtgaaaatattgttgatgcgctggcagtgttcctgcgc

SEQ ID NO: 44 - DNA, 45 bp, synthetic construct
  source 1..45: mol_type = other DNA
  misc_feature 1..45: note = Sequence of the 3' region of the G418 resistance gene to be recombined via V(D)J in the pY112-CJA-U-H0opt plasmid
cggttgcattcgattcctgtttgtaattgtccttttaacagcgat

SEQ ID NO: 45 - protein, 692 aa, synthetic construct
  REGION 1..692: note = MRE11
MDYPDPDTIRILITTDNHVGYNENDPITGDDSWKTFHEVMMLAKNNNVDMVVQSGDLFHVNKPSKKSLYQVLKTLRLCCMGDKPCELELLSDPSQVFHYDEFTNVNYEDPNFNISIPVFGISGNHDDASGDSLLCPMDILHATGLINHFGKVIESDKIKVVPLLFQKGSTKLALYGLAAVRDERLFRTFKDGGVTFEVPTMREGEWFNLMCVHQNHTGHTNTAFLPEQFLPDFLDMVIWGHEHECIPNLVHNPIKNFDVLQPGSSVATSLCEAEAQPKYVFILDIKYGEAPKMTPIPLETIRTFKMKSISLQDVPHLRPHDKDATSKYLIEQVEEMIRDANEETKQKLADDGEGDMVAELPKPLIRLRVDYSAPSNTQSPIDYQVENPRRFSNRFVGRVANGNNVVQFYKKRSPVTRSKKSGINGTSISDRDVEKLFSESGGELEVQTLVNDLLNKMQLSLLPEVGLNEAVKKFVDKDEKTALKEFISHEISNEVGILSTNEEFLRTDDAEEMKALIKQVKRANSVRPTPPKENDETNFAFNGNGLDSFRSSNREVRTGSPDITQSHVDNESRITHISQAESSKPTSKPKRVRTATKKKIPAFSDSTVISDAENELGDNNDAQDDVDIDENDIIMVSTDEEDASYGLLNGRKTKTKTRPAASTKTASRRGKGRASRTPKTDILGSLLAKKRK

SEQ ID NO: 46 - protein, 1312 aa, synthetic construct
  REGION 1..1312: note = RAD50
MSAIYKLSIQGIRSFDSNDRETIEFGKPLTLIVGMNGSGKTTIIECLKYATTGDLPPNSKGGVFIHDPKITGEKDIRAQVKLAFTSANGLNMIVTRNIQLLMKKTTTTFKTLEGQLVAINNSGDRSTLSTRSLELDAQVPLYLGVPKAILEYVIFCHQEDSLWPLSEPSNLKKKFDEIFQAMKFTKALDNLKSIKKDMSVDIKLLKQSVEHLKLDKDRSKAMKLNIHQLQTKIDQYNEEVSEIESQLNEITEKSDKLFKSNQDFQKILSKVENLKNTKLSISDQVKRLSNSIDILDLSKPDLQNLLANFSKVLMDKNNQLRDLETDISSLKDRQSSLQSLSNSLIRRQGELEAGKETYEKNRNHLSSLKEAFQHKFQGLSNIENSDMAQVNHEMSQFKAFISQDLTDTIDQFAKDIQLKETNLSDLIKSITVDSQNLEYNKKDRSKLIHDSEELAEKLKSFKSLSTQDSLNHELENLKTYKEKLQSWESENIIPKLNQKIEEKNNEMIILENQIEKFQDRIMKTNQQADLYAKLGLIKKSINTKLDELQKITEKLQNDSRIRQVFPLTQEFQRADLEMDFQKLFINMQKNIAINNKKMHELDRRYTNALYNLNTIEKDLQDNQKSKEKVIQLLSENLPEDCTIDEYNDVLEETELSYKTALENLKMHQTTLEFNRKALEIAERDSCCYLCSRKFENESFKSKLLQELKTKTDANFEKTLKDTVQNEKEYLHSLRLLEKHIITLNSINEKIDNSQKCLEKAKEETKTSKSKLDELEVDSTKLKDEKELAESEIRPLIEKFTYLEKELKDLENSSKTISEELSIYNTSEDGIQTVDELRDQQRKMNDSLRELRKTISDLQMEKDEKVRENSRMINLIKEKELTVSEIESSLTQKQNIDDSIRSKRENINDIDSRVKELEARIISLKNKKDEAQSVLDKVKNERDIQVRNKQKTVADINRLIDRFQTIYNEVVDFEAKGFDELQTTIKELELNKAQMLELKEQLDLKSNEVNEEKRKLADSNNEEKNLKQNLELIELKSQLQHIESEISRLDVQNAEAERDKYQEESLRLRTRFEKLSSENAGKLGEMKQLQNQIDSLTHQLRTDYKDIEKNYHKEWVELQTRSFVTDDIDVYSKALDSAIMKYHGLKMQDINRIIDELWKRTYSGTDIDTIKIRSDEVSSTVKGKSYNYRVVMYKQDVELDMRGRCSAGQKVLASIIIRLALSETFGANCGVIALDEPTTNLDEENIESLAKSLHNIINMRRHQKNFQLIVITHDEKFLGHMNAAAFTDHFFKVKRDDRQKSQIEWVDINRVTY

SEQ ID NO: 47 - protein, 854 aa, synthetic construct
  REGION 1..854: note = XRS2
MWVVRYQNTLEDGSISFISCCLQAFKTYSIGRSSKNPLIIKNDKSISRQHITFKWEINNSSDLKHSSLCLVNKGKLTSLNKKFMKVGETFTINASDVLKSTIIELGTTPIRIEFEWINEVWNIPPHLTQFRTMLSEYGISTEISINDIPANLMISDYPKSEDNSIRELYALVSTIPMKKSRFLMELCNTLLPTSKTNLKFDEMWNDMISNPEYNVFDFDPNILLSKFMRLNNIRVLTTIKSEPRLSSLLRTFNINLFAFDNIDSLYKYVDSLEASTEYLILTTTDKKENGKILCTIKTMLTSIIDGTLSAVINMKGASSRTLDNGKFDQISEGMSTILKTSRAPEVEASPVVSKKRKLNRRRVLPLDSLDFFAGGLSTKTLSENRSLTDAKRLNCGAESKTVISSPNIAEADEKHAPFLQNALKPTEDIGKKSGHSSPGAIIVSSPNLGTVNTSEDSLDKSLQSHKLPQPSLPEVAGIGSQTISSNSADYETAAVNSMDDAEVTKNFRVNHHQNIEQPSKNIRKLSNYSREISSPLQENCKSPVKELSIKEKSGTPHAFVEAIQETKNREVKRVKSTIVELKDEELSEEAINQLKNLAIVEPSNNLLRKSFDSEGNKTSRTTEKWENSLMEPEWHKRKNFKTFVKVRPKSKAHKEEGKNNTQSSDFIRNAAFLITRNYVPLKKYSKKDTTTKWGTEENEDMFALTEMERFGSNTFMSDNINSNTIQKRSQALNRFTNEDSSNEGEEDSFSFSRCSGTAASVQPLKNKIFITDEDDLGDIDDKSDRLNHRENNRNLFVVKEMNLRPNLSEECSKQSRHSRSATSRSRGSFGASNNGDGDDDDDDGPKFTFKRRKG

SEQ ID NO: 48 - DNA, 6941 bp, synthetic construct
  source 1..6941: mol_type = other DNA
  misc_feature 1..6941: note = pY112-CJA-U-H0opt
gaggtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgtttaagccaagatttccttgacagccttggcgatagcatcgccaacctcagtggtagagttagaaccaccaaggtcaccggttctgacacctgcatccaagacatttctaacagcttcttcaagagccctaccttcttcaaccaaatccaaggataacttcaacatcatagctgcagataagatggtagcaattgggttaaccttgtttgctggtaaatctggggcagaaccatgacatggttcgtacaaaccgaatgccttgttagtgtcaggtagggaagctagagatgcagaaggtaataaacccaaagaacctggaataacagaggcttcatcggagataatatcaccaaacatgttgttggtaataacaacaccgtttagcttagttggtgatttaaccaaaatcatagcagcagaatcgatcaattggtgctgaacagttaattgtgggaactcagtcttgatggtttcttcaacagtctttctccacaatctggaagaggcaagcacgttagccttgtcaagtgaccagattggtaatggtgggttttgttgcaatgccaagaaagcagccattcttgtaattctttgaacttcaggaacactgtatttctcagaatcccaagcaactccgtcaccttcatcttcttttctttcaccaaagtagataccaccaaccaattctctaacgacgacgaaatcggtaccctttgcatattcaggcttcaaaggagaaagatctagtaaagaatcagaagcaaagttacatggcctcaagttggcgtatagacccaattcctttctgatcttcaatagaccttgttctggtctaactgcgcccgtaccccattttggaccaccaacagcacctagtaagacagcatcggctttcttagaggcttctagagcttcatctggtaaaggagtgccagtggcatcgatggcagcacccccgatcaagtgatgttggaaattgaacttaatttctggacggacttcagcaatggcattcaagaccttaatagcttcgtcagtaacttctttaccgacgtgatcacccggtaggacaacgatattcttagacattgttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacaaggcaagctgcacctagatccttttaaattaaaaatgaagttttaaatcaatctaaagtatatatgagtaaacttggtctgacagagttctgaggtcattactggatctatcaacagcagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgcgcgtttgccacctgacgtctaagaaaaggaatattcagcaatttgcccgtgccgaagaaaggcccacccgtgaaggtgagccagtgagttgattggaagacctgtgcgtaatgcttcaaaaacgcactgtactcctttttactcttccggattttctcgcactctccgcatcgccgcacgagccaagccacacccacacacctcataccatgtttcccctctttgtctctttcgtgcggctccattacccgcatgaaactgtataaaagtaacaaaagactatttcgtttctttttctttgtcggaaaaggcaaaaaaaaaaatttttatcacatttctttttcttgaaaattttttttgggattttttctctttcgatgacctcccattgatatttaagttaataaaagcactcccgttttccaagttttaatttgttcctcttgtttagtcattcttcttctcagcattggtcaattagaaagagagcatagcaaactgatctaagttttaattacaaaatgggtaaggaaaagacacacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccacagtgctacagactggaacaaaaacccaatacaacagatcacgtgatctttttgtaagatgaagttgaagtagtgttgcaccgtgccaatgcaggtggctattagattaaatatgtgatttgttctattaagtttcctgtataatttaatggggagcgctgattctcttttggtacgcttcccatccagcatttctgtatctttcaccttcaaccttaggatctctacccttggcgaaaagtcctctgccaacaatgatgatatctgatccaccacttacaacttcgtcgacggttctgtactgctgacccaatgcatcgcctttgtcgtctaaacctacacctggggtcatgattagccaatcaaacccttcttctcttcctcccatatcgttctgagcaatgaacccaataacgaaatctttatcactctttgcaatatcaacggtacccttagtatattcaccgtgtgctagagaacccttggatgacaattcagcaagcatcaataatccccttggttctttggtgacctcttgcgcaccttgtttcaagccagcaacaataccagcaccagtaaccccgtgggcgttggtgatatcagaccattctgcgatacggtaaacgcccgatgtatattgtaatttgactgtgttaccgatatcggcgaattttctgtcctcaaatatcaagaacttgtatttctctgccaatgctttcaatggaacgacagtaccctcataactgaaatcatccaagatatcaacgtgtgttttcaaaaggcaaatgtatggacccaacgtttcaacaagtttcaatagctcatcagtcgaacgaacgtcaagagaagcacacaaattggttttcttttcatccattaaacgtaaaagtttcgatgcaaccggacttgcatgtgtctcagctctactggtatatgattttgtggacattttgaatatgtattacttggttatggttatatatgacaaaagaaaaagaagaacagaagaataacgcaaggaagaacaataactgaaattgatagagaagtattatgtctttgtctttttataataaatcaagtgcagaaatccgttagacaacatgagggataaaatttaacgtgggcgaagaagaaggaaaaaagtttttgtgagggcgtaattgaagcgatctgttgattgtagattttttttttttgaggagacaaagtcagaagagaacagacaaatggtattaaccatccaatacttttttggagcaacgctaagctcatgcttttccattggttacgtgctcagttgttagatatggaaagagaggatgctcacggcagcgtgacaccaattgagcccgaaagagaggatgccacgttttcccgacggctgctagaatggaaaaaggaaaaatagaagaatcccattcctatcattatttacgtaatgacccacacatttttgagattttcaactattacgtattacgataatcctgctgtcatggtttttgtacagccagacagtggagtactaccactgtgcggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgtgtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaatttgatctgtagcctaagtataaaattctacgtatgtatatatttacatgcaattttttctttttccaattcatggtacctcaggtcttcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagcccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacgtgcggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcatctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaatg